(19) United States
(12) Patent Application Publication
(10) Pub. No.: US 2023/0404642 A1
(43) Pub. Date: Dec. 21, 2023

(54) METHOD FOR THE TREATMENT OF CANCER VIA TUMOR CELL LYSIS AND INTRATUMORAL ADMINISTRATION OF COMBINATIONS OF IMMUNOTHERAPEUTIC INGREDIENTS

(71) Applicant: SYNCROMUNE, INC., Fort Lauderdale, FL (US)

(72) Inventors: Charles J. Link, JR., Fort Lauderdale, FL (US); Jason R. Williams, Fort Lauderdale, FL (US); Mario R. Mautino, Fort Lauderdale, FL (US); Daniel K. Recinelia, Fort Lauderdale, FL (US); Eamonn P. Hobbs, Fort Lauderdale, FL (US)

(21) Appl. No.: 18/204,779

(22) Filed: Jun. 1, 2023

Related U.S. Application Data
(60) Provisional application No. 63/347,690, filed on Jun. 1, 2022.

(51) Int. Cl. A61B 18/02; C07K 16/28; C07K 14/00; A61P 35/00

(52) U.S. Cl. CPC A61B 18/02; C07K 16/2878; C07K 16/2818; C07K 14/001; A61P 35/00; A61B 2018/0293; A61K 2039/507

(57) ABSTRACT

The present disclosure provides, among other things, methods of cancer treatment comprising steps of: a) intratumoral cell lysis, mediated by cryolysis; and b) intratumoral administration of 1) a combination of immunotherapeutic agents comprising: i) a TLR9 agonist CpG oligodeoxydinucleotide, ii) an agonistic anti-CD40 monoclonal antibody, iii) an agonistic anti-OX40 monoclonal antibody and iv) an anti-CTLA4 monoclonal antibody; or 2) a combination of immunotherapeutic agents comprising: i) a TLR9 agonist CpG oligodeoxydinucleotide, ii) an agonistic anti-CD40 monoclonal antibody, iii) an anti-PD1 monoclonal antibody and iv) an anti-CTLA4 monoclonal antibody.

### CROSS REFERENCE TO RELATED APPLICATIONS

This application claims the benefit of and priority to U.S. Provisional Patent Application No. 63/347,690, filed Jun. 1, 2022, which is incorporated herein by reference in its entirety for all purposes.

### REFERENCE TO SEQUENCE LISTING

This application contains a Sequence Listing which has been submitted electronically in XML format and is hereby incorporated by reference in its entirety. Said Sequence Listing XML, created on Sep. 5, 2023, is named SYNC-001_SL.xml, and is 35,703 bytes in size.

### BACKGROUND

Current clinical approaches for cancer immunotherapy rely on the systemic administration of high doses of one or two biological immunomodulatory agents. These agents aim at reverting the pre-established conditions of tumor-induced immunosuppression while stimulating an adaptive immune response against tumor-specific antigens (TSA) and tumor-associated antigens (TAA).

Major advances in the treatment of cancer have been made in the last decade by the use of antagonistic monoclonal antibodies (mAbs) that block immune checkpoint inhibitor molecules such as PD-1, PD-L1 and CTLA-4. However, despite the significant benefits obtained by the use of these mAbs, a substantial fraction of patients still do not respond to the therapy or become refractory to it. Additionally, the use of high systemic doses of these checkpoint inhibitors is accompanied by high frequency of serious adverse events, particularly when used in combination with other immunotherapeutic agents, which limits the number of agents and doses that can be combined to address the multifactorial nature of tumor immune resistance (Vilgelm, Johnson et al. 2016). There is a need in the art for new approaches to cancer immunotherapy.

### SUMMARY

Current cancer immunotherapeutic approaches only provide benefit to a limited fraction of patients and their use often results in partial responses of limited duration. Furthermore, current cancer immunotherapeutic approaches are associated with treatment-acquired resistance and a high percentage of immune adverse events which limit their compliance and applicability. The present disclosure sets forth methods and compositions for treating cancer that overcome the many limitations of current cancer immunotherapeutic approaches.

In some embodiments, the immunotherapeutic methods of the present disclosure are not antigen-specific and do not require prior antigen identification. Methods of the present disclosure, in many embodiments, comprise a combination of different pharmacologic agents that mediate different and synergistic antitumor immune activation effects, thus increasing efficacy and reducing the possibility of immune acquired resistance compared to therapies relying on a single therapeutic agent. Moreover, use of an intratumoral route of administration for compositions of the present disclosure mediates a loco-regional treatment that increases drug accessibility and drug biodistribution to immune relevant tumor associated immune cells and associated secondary and tertiary lymphoid tissues. Additionally, intratumoral methods of administration, as described herein, allow for a significant reduction in dose, which reduces the probability of toxicity and adverse events. This allows for increasing the number of therapeutic agents in drug combinations of the present disclosure, thus increasing the breadth of pharmacologic effects to increase antitumor efficacy. The increase in therapeutic window achieved by use of lower doses and the increase in the number of therapeutic agents with synergistic mechanism of action increases the efficacy of treatment while reducing the frequency and severity of adverse events. Thus, the present disclosure provides, among other things, methods for the treatment of cancer that overcome the limitations of current cancer immunotherapeutic options.

The present disclosure further provides combinations and compositions of immunotherapeutic agents that, at least, mediate (1) powerful activation of the innate arm of an immune response to mediate local antitumor effects; (2) the activation of the adaptive arm of an antitumor immune response by recruitment of antigen presenting cells, activation and maturation of APCs; (3) activation, proliferation and increased survival of T cells; (4) reduction of the immunosuppressive effects of regulatory T cells; and/or (5) reversion and prevention of T cell exhaustion by factors that dominate a suppressive tumor microenvironment.

The present disclosure also provides doses for different immunotherapeutic agents present in compositions and formulations as described herein. Indeed, the present disclosure further provides, among other things, compositions for the treatment of cancer that overcome the limitations of current cancer immunotherapeutic options.

For example, in some embodiments, the present disclosure provides a method for the treatment of cancer, whereby such method comprises a first step aimed at initiating in situ immunization by triggering lysis and immunogenic cell death of tumor cells, using an intratumoral cryolysis device; and a second step comprising intratumoral administration of a combination of immunotherapeutic agents that mediate different pharmacologic effects. In some embodiments, combinations of immunotherapeutic agents provided mediate synergistic pharmacologic effects when administered to a subject.

Further, in some embodiments, the present disclosure provides methods and compositions including immunotherapeutic combinations of active ingredients that are dosed by intratumoral administration. In some embodiments, a composition, or combination formulation, comprises a TLR9 agonist, an anti-CD40 agonistic monoclonal antibody, an anti-OX40 agonistic monoclonal antibody, and an anti-CTLA4 monoclonal antibody. In some embodiments, a composition, or combination formulation, consists of a TLR9 agonist, an anti-CD40 agonistic monoclonal antibody, an anti-OX40 agonistic monoclonal antibody, and an anti-CTLA4 monoclonal antibody. In some embodiments, a composition, or combination formulation, comprises a TLR9 agonist, an anti-CD40 agonistic monoclonal antibody, an anti-PD1 monoclonal antibody, and an anti-CTLA4 monoclonal antibody. In some embodiments, a composition, or combination formulation, consists of a TLR9 agonist, an anti-CD40 agonistic monoclonal antibody, an anti-PD1 monoclonal antibody, and an anti-CTLA4 monoclonal antibody.

Additionally, in some embodiments, the present disclosure provides dose ranges for each active ingredient for different combinations of drug preparations disclosed herein.

The present disclosure provides methods of treating cancer in a subject, the methods comprising steps of: a) administering to a tumor in the subject a therapy that causes tumor cell lysis; and b) administering to the subject a therapeutically effective amount of a combination formulation, wherein the combination formulation comprises: a. a TLR9 agonist; b. an agonistic anti-CD40 monoclonal antibody; c. an anti-CTLA4 monoclonal antibody; and d. an agonistic anti-OX40 monoclonal antibody or an anti-PD1 monoclonal antibody.

In some embodiments, a combination formulation comprises: a. a TLR9 agonist; b. an agonistic anti-CD40 monoclonal antibody; c. an agonistic anti-OX40 monoclonal antibody; and d. an anti-CTLA4 monoclonal antibody.

In some embodiments, a combination formulation consists of: a. a TLR9 agonist; b. an agonistic anti-CD40 monoclonal antibody; c. an agonistic anti-OX40 monoclonal antibody; and d. an anti-CTLA4 monoclonal antibody.

In some embodiments, a combination formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 1; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26;

In some embodiments, a combination formulation comprises a TLR9 agonist as set forth in SEQ ID NO: 2; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an combination formulation comprises a TLR9 agonist as set forth in SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29; an agonistic anti-OX40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31; and an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, a combination formulation comprises a TLR9 agonist as set forth in SEQ ID NO: 1; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, a combination formulation comprises a TLR9 agonist as set forth in SEQ ID NO: 2; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, a combination formulation comprises a TLR9 agonist as set forth in SEQ ID NO: 27; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, a combination formulation comprises: a. a TLR9 agonist; b. an agonistic anti-CD40 monoclonal antibody; c. an anti-PD1 monoclonal antibody; and d. an anti-CTLA4 monoclonal antibody.

In some embodiments, a combination formulation consists of: a. a TLR9 agonist; b. an agonistic anti-CD40 monoclonal antibody; c. an anti-PD1 monoclonal antibody; and d. an anti-CTLA4 monoclonal antibody.

In some embodiments, a combination formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 1; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 2; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a combination formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29; an anti-PD1 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33; and an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, a combination formulation comprises a TLR9 agonist as set forth in SEQ ID NO: 1; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, a combination formulation comprises a TLR9 agonist as set forth in SEQ ID NO: 2; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, a combination formulation comprises a TLR9 agonist as set forth in SEQ ID NO: 27; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, a TLR9 agonist, an agonistic anti-CD40 monoclonal antibody, an agonistic anti-OX40 monoclonal antibody, and an anti-CTLA4 monoclonal antibody are co-administered.

In some embodiments, a TLR9 agonist, an agonistic anti-CD40 monoclonal antibody, an agonistic anti-OX40 monoclonal antibody, and an anti-CTLA4 monoclonal antibody are administered sequentially.

In some embodiments, a TLR9 agonist is administered in a separate composition sequentially with a composition comprising an agonistic anti-CD40 monoclonal antibody, an agonistic anti-OX40 monoclonal antibody, and an anti-CTLA4 monoclonal antibody.

In some embodiments, a TLR9 agonist, an agonistic anti-CD40 monoclonal antibody, an anti-PD1 monoclonal antibody, and an anti-CTLA4 monoclonal antibody are co-administered.

In some embodiments, a TLR9 agonist, an agonistic anti-CD40 monoclonal antibody, an anti-PD1 monoclonal antibody, and an anti-CTLA4 monoclonal antibody are administered sequentially.

In some embodiments, a TLR9 agonist is administered in a separate composition sequentially with a composition comprising an agonistic anti-CD40 monoclonal antibody, an anti-PD1 monoclonal antibody, and an anti-CTLA4 monoclonal antibody.

In some embodiments, a therapy administered to a tumor is a cryolysis therapy that mediates tumor cell lysis by cryolysis.

In some embodiments, administration of a cryolysis therapy comprises at least one, at least two, or at least three cycles of freeze-thaw. In some embodiments, administration of a cryolysis therapy comprises one cycle of freeze-thaw. In some embodiments, administration of a cryolysis therapy comprises two cycles of freeze-thaw. In some embodiments, administration of a cryolysis therapy comprises three cycles of freeze-thaw.

In some embodiments, administration of a cryolysis therapy comprises no more than one, no more than two, or no more than three cycles of freeze-thaw. In some embodiments, administration of a cryolysis therapy comprises no more than one cycle of freeze-thaw. In some embodiments, administration of a cryolysis therapy comprises no more than two cycles of freeze-thaw. In some embodiments, a cryolysis therapy comprises no more than three cycles of freeze-thaw.

In some embodiments, a cryolysis therapy is mediated by an intratumoral cryolysis device comprising a cryoprobe for contacting and freezing tumor cells during freeze-thaw cycles. In some embodiments, a cryogen circulates within a cryoprobe. In some embodiments, a cryogen is selected from the group consisting of: argon, nitrous oxide, carbon dioxide, and liquid nitrogen.

In some embodiments, an intratumoral cryolysis device is set to a duty cycle of about 50%, about 60%, about 70%, about 80%, about 90%, or about 100% and is operated during freeze-thaw cycles to generate a cryoprobe temperature of about −40° C. or colder, about −45° C. or colder, about −50° C. or colder, about −55° C. or colder, about −60° C. or colder, about −65° C. or colder, about −70° C. or colder, about −75° C. or colder, about −80° C. or colder, about −85° C. or colder, about −90° C. or colder, about −100° C. or colder, about −110° C. or colder, or about −120° C. or colder in less than about one minute followed by a step of passive thawing. In some embodiments, an intratumoral cryolysis device is set to a duty cycle of about 50%, about 60%, about 70%, about 80%, about 90%, or about 100% and is operated during freeze-thaw cycles to generate a cryoprobe temperature of about −40° C. or colder, about −45° C. or colder, about −50° C. or colder, about −55° C. or colder, about −60° C. or colder, about −65° C. or colder, about −70° C. or colder, about −75° C. or colder, about −80° C. or colder, about −85° C. or colder, about −90° C. or colder, about −100° C. or colder, about −110° C. or colder, or about −120° C. or colder in less than about 30 seconds followed by a step of passive thawing. In some embodiments, an intratumoral cryolysis device is set to a duty cycle of about 70% to about 100% and is operated during freeze-thaw cycles to generate a cryoprobe temperature of about −40° C. to about −60° C. in less than about one minute followed by a step of passive thawing. In some embodiments, an intratumoral cryolysis device is set to a duty cycle of about 70% to about 100% and is operated during freeze-thaw cycles to generate a cryoprobe temperature of about −40° C. to about −60° C. in less than about 30 seconds followed by a step of passive thawing. In some embodiments, an intratumoral cryolysis device is set to 100% duty cycle and is operated during freeze-thaw cycles to generate a cryoprobe temperature of −40° C. or colder in less than about one minute followed by a step of passive thawing. In some embodiments, a generated cryoprobe temperature is maintained for about one minute before passive thawing. In some embodiments, a generated cryoprobe temperature is maintained for about two minutes before passive thawing. In some embodiments, a generated cryoprobe temperature is maintained for about three minutes before passive thawing. In some embodiments, a generated cryoprobe temperature continues to cool to a temperature of about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder for a total time of about 5 minutes, about 4.5 minutes, about 4 minutes, about 3.5 minutes, about 3 minutes, about 2.5 minutes, about 2 minutes, about 1.75 minutes, about 1.5 minutes, about 1.25 minutes, about 1 minute, about 50 seconds, about 40 seconds, about 30 seconds, about 20 seconds, or about 10 seconds.

In some embodiments, a cryoprobe is cooled to a temperature that creates an ice ball within a tumor that has a diameter of about 8 mm to about 30 mm, about 10 mm to about 30 mm, about 15 mm to about 30 mm, about 20 mm to about 30 mm, or about 25 mm to about 30 mm, about 8 mm to about 25 mm, about 8 mm to about 20 mm, about 8 mm to about 15 mm, about 8 mm to about 10 mm, about 10 mm to about 18 mm, or about 12 mm to about 16 mm. In some embodiments, a cryoprobe is cooled to a temperature that creates an ice ball within a tumor that has a diameter of about 12 mm to about 16 mm. In some particularly preferred embodiments, a cryoprobe is cooled to a temperature that creates an ice ball within a tumor that has a diameter of about 14 mm.

In some embodiments, a majority of an ice ball has a temperature of approximately −40° C. In some embodiments, a majority of an ice ball has a temperature of approximately −50° C. In some embodiments, a majority of an ice ball has a temperature of approximately −60° C. In some embodiments, a majority of an ice ball has a temperature of approximately −40° C. or colder. In some embodiments, a majority of an ice ball has a temperature of approximately −50° C. or colder. In some embodiments, a majority of an ice ball has a temperature of approximately −60° C. or colder. In some embodiments, a majority of an ice ball has a temperature of approximately −40° C. to approximately −60° C.

In some embodiments, an ice ball comprises an approximately 1 cm (10 mm) diameter region within that has a temperature of approximately −40° C. In some embodiments, an ice ball comprises an approximately 1 cm diameter region within that has a temperature of approximately −50° C. In some embodiments, an ice ball comprises an approximately 1 cm diameter region within that has a temperature of approximately −60° C. In some embodiments, an ice ball comprises an approximately 1 cm diameter region within that has a temperature of approximately −40° C. or colder. In some embodiments, an ice ball comprises an approximately 1 cm diameter region within that has a temperature of approximately −50° C. or colder. In some embodiments, an ice ball comprises an approximately 1 cm diameter region within that has a temperature of approximately −60° C. or colder. In some embodiments, an ice ball comprises an approximately 0.5 cm to approximately 1.5 cm diameter region within that has a temperature of approximately −40° C. In some embodiments, an ice ball comprises an approximately 0.5 cm to approximately 1.5 cm diameter region within that has a temperature of approximately −50° C. In some embodiments, an ice ball comprises an approximately 0.5 cm to approximately 1.5 cm diameter region within that has a temperature of approximately −60° C. In some embodiments, an ice ball comprises an approximately 0.5 cm to approximately 1.5 cm diameter region within that has a temperature of approximately −40° C. or colder. In some embodiments, an ice ball comprises an approximately 0.5 cm to approximately 1.5 cm diameter region within that has a temperature of approximately −50° C. or colder. In some embodiments, an ice ball comprises an approximately 0.5 cm to approximately 1.5 cm diameter region within that has a temperature of approximately −60° C. or colder.

In some embodiments, a cryolysis therapy does not ablate an entire tumor but causes partial tumor cell lysis.

In some embodiments, a cryolysis therapy causes necrotic cell death in a zone of tumor tissue with the zone being approximately 14 mm in diameter. In some embodiments, a cryolysis therapy causes necrotic cell death in a zone of tumor tissue with the zone being approximately 8 mm to approximately 30 mm, approximately 10 mm to approximately 30 mm, approximately 15 mm to approximately 30 mm, approximately 20 mm to approximately 30 mm, approximately 25 mm to approximately 30 mm, approximately 8 mm to approximately 25 mm, approximately 8 mm to approximately 20 mm, approximately 8 mm to approximately 15 mm, approximately 8 mm to approximately 10 mm, approximately 10 mm to approximately 18 mm, or approximately 12 mm to approximately 16 mm.

In some embodiments, a TLR9 agonist is a CpG ODN of class B or C. In some embodiments, TLR9 agonist is a CpG ODN of class B. In some embodiments, a TLR9 agonist is a CpG ODN of class C.

In some embodiments, a TLR9 agonist comprises a nucleic acid sequence as set forth in SEQ ID NO: 1, SEQ ID NO: 2, or SEQ ID NO: 27. In some embodiments, a TLR9 agonist is a nucleic acid as set forth in SEQ ID NO: 1, SEQ ID NO: 2, or SEQ ID NO: 27. In some embodiments, a TLR9 agonist is a nucleic acid as set forth in SEQ ID NO: 1. In some embodiments, a TLR9 agonist is a nucleic acid as set forth in SEQ ID NO: 2. In some embodiments, a TLR9 agonist is a nucleic acid as set forth in SEQ ID NO: 27.

In some embodiments, an agonistic anti-CD40 monoclonal antibody comprises a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8. In some embodiments of the present disclosure, an agonistic anti-CD40 monoclonal antibody is of human IgG2k isotype.

In some embodiments of the present disclosure, an agonistic anti-CD40 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28. In some embodiments of the present disclosure, an agonistic anti-CD40 monoclonal antibody comprises a heavy chain, wherein the heavy chain comprises an amino acid sequence having at least 90% sequence identity to SEQ ID NO: 28. In some embodiments of the present disclosure, an agonistic anti-CD40 monoclonal antibody comprises a heavy chain, wherein the heavy chain comprises an amino acid sequence having at least 95% sequence identity to SEQ ID NO: 28. In some embodiments of the present disclosure, an agonistic anti-CD40 monoclonal antibody comprises a heavy chain, wherein the heavy chain comprises an amino acid sequence having at least 96% sequence identity to SEQ ID NO: 28. In some embodiments of the present disclosure, an agonistic anti-CD40 monoclonal antibody comprises a heavy chain, wherein the heavy chain comprises an amino acid sequence having at least 97% sequence identity to SEQ ID NO: 28. In some embodiments of the present disclosure, an agonistic anti-CD40 monoclonal antibody comprises a heavy chain, wherein the heavy chain comprises an amino acid sequence having at least 98% sequence identity to SEQ ID NO: 28. In some embodiments of the present disclosure, an agonistic anti-CD40 monoclonal antibody comprises a heavy chain, wherein the heavy chain comprises an amino acid sequence having at least 99% sequence identity to SEQ ID NO: 28. In some embodiments of the present disclosure, an agonistic anti-CD40 monoclonal antibody comprises a heavy chain, wherein the heavy chain comprises an amino acid sequence as set forth in SEQ ID NO: 28.

In some embodiments of the present disclosure, an agonistic anti-CD40 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments of the present disclosure, an agonistic anti-CD40 monoclonal antibody comprises a light chain, wherein the light chain comprises an amino acid sequence having at least 90% sequence identity to SEQ ID NO: 29. In some embodiments of the present disclosure, an agonistic anti-CD40 monoclonal antibody comprises a light chain, wherein the light chain comprises an amino acid sequence having at least 95% sequence identity to SEQ ID NO: 29. In some embodiments of the present disclosure, an agonistic anti-CD40 monoclonal antibody comprises a light chain, wherein the light chain comprises an amino acid sequence having at least 96% sequence identity to SEQ ID NO: 29. In some embodiments of the present disclosure, an agonistic anti-CD40 monoclonal antibody comprises a light chain, wherein the light chain comprises an amino acid sequence having at least 97% sequence identity to SEQ ID NO: 29. In some embodiments of the present disclosure, an agonistic anti-CD40 monoclonal antibody comprises a light chain, wherein the light chain comprises an amino acid sequence having at least 98% sequence identity to SEQ ID NO: 29. In some embodiments of the present disclosure, an agonistic anti-CD40 monoclonal antibody comprises a light chain, wherein the light chain comprises an amino acid sequence having at least 99% sequence identity to SEQ ID NO: 29. In some embodiments of the present disclosure, an agonistic anti-CD40 monoclonal antibody comprises a light chain, wherein the light chain comprises an amino acid sequence as set forth in SEQ ID NO: 29.

In some embodiments of the present disclosure, an agonistic anti-CD40 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 90% sequence identity to SEQ ID NO: 28, and the light chain comprises an amino acid sequence having at least 90% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 95% sequence identity to SEQ ID NO: 28, and the light chain comprises an amino acid sequence having at least 95% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 96% sequence identity to SEQ ID NO: 28, and the light chain comprises an amino acid sequence having at least 96% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 97% sequence identity to SEQ ID NO: 28, and the light chain comprises an amino acid sequence having at least 97% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 98% sequence identity to SEQ ID NO: 28, and the light chain comprises an amino acid sequence having at least 98% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 99% sequence identity to SEQ ID NO: 28, and the light chain comprises an amino acid sequence having at least 99% sequence identity to SEQ ID NO: 29. In some embodiments of the present disclosure, an agonistic anti-CD40 monoclonal antibody comprises a heavy chain and a light chain, wherein the heavy chain comprises an amino acid sequence as set forth in SEQ ID NO: 28, and the light chain comprises an amino acid sequence as set forth in SEQ ID NO: 29.

In some embodiments, an agonistic anti-OX40 monoclonal antibody comprises a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14. In some embodiments of the present disclosure, an agonistic anti-OX40 monoclonal antibody is of human IgG1k isotype.

In some embodiments of the present disclosure, an agonistic anti-OX40 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30. In some embodiments of the present disclosure, an agonistic anti-OX40 monoclonal antibody comprises a heavy chain, wherein the heavy chain comprises an amino acid sequence having at least 90% sequence identity to SEQ ID NO: 30. In some embodiments of the present disclosure, an agonistic anti-OX40 monoclonal antibody comprises a heavy chain, wherein the heavy chain comprises an amino acid sequence having at least 95% sequence identity to SEQ ID NO: 30. In some embodiments of the present disclosure, an agonistic anti-OX40 monoclonal antibody comprises a heavy chain, wherein the heavy chain comprises an amino acid sequence having at least 96% sequence identity to SEQ ID NO: 30. In some embodiments of the present disclosure, an agonistic anti-OX40 monoclonal antibody comprises a heavy chain, wherein the heavy chain comprises an amino acid sequence having at least 97% sequence identity to SEQ ID NO: 30. In some embodiments of the present disclosure, an agonistic anti-OX40 monoclonal antibody comprises a heavy chain, wherein the heavy chain comprises an amino acid sequence having at least 98% sequence identity to SEQ ID NO: 30. In some embodiments of the present disclosure, an agonistic anti-OX40 monoclonal antibody comprises a heavy chain, wherein the heavy chain comprises an amino acid sequence having at least 99% sequence identity to SEQ ID NO: 30. In some embodiments of the present disclosure, an agonistic anti-OX40 monoclonal antibody comprises a heavy chain, wherein the heavy chain comprises an amino acid sequence as set forth in SEQ ID NO: 30.

In some embodiments of the present disclosure, an agonistic anti-OX40 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31. In some embodiments of the present disclosure, an agonistic anti-OX40 monoclonal antibody comprises a light chain, wherein the light chain comprises an amino acid sequence having at least 90% sequence identity to SEQ ID NO: 31. In some embodiments of the present disclosure, an agonistic anti-OX40 monoclonal antibody comprises a light chain, wherein the light chain comprises an amino acid sequence having at least 95% sequence identity to SEQ ID NO: 31. In some embodiments of the present disclosure, an agonistic anti-OX40 monoclonal antibody comprises a light chain, wherein the light chain comprises an amino acid sequence having at least 96% sequence identity to SEQ ID NO: 31. In some embodiments of the present disclosure, an agonistic anti-OX40 monoclonal antibody comprises a light chain, wherein the light chain comprises an amino acid sequence having at least 97% sequence identity to SEQ ID NO: 31. In some embodiments of the present disclosure, an agonistic anti-OX40 monoclonal antibody comprises a light chain, wherein the light chain comprises an amino acid sequence having at least 98% sequence identity to SEQ ID NO: 31. In some embodiments of the present disclosure, an agonistic anti-OX40 monoclonal antibody comprises a light chain, wherein the light chain comprises an amino acid sequence having at least 99% sequence identity to SEQ ID NO: 31. In some embodiments of the present disclosure, an agonistic anti-OX40 monoclonal antibody comprises a light chain, wherein the light chain comprises an amino acid sequence as set forth in SEQ ID NO: 31.

In some embodiments of the present disclosure, an agonistic anti-OX40 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 90% sequence identity to SEQ ID NO: 30, and the light chain comprises an amino acid sequence having at least 90% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 95% sequence identity to SEQ ID NO: 30, and the light chain comprises an amino acid sequence having at least 95% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 96% sequence identity to SEQ ID NO: 30, and the light chain comprises an amino acid sequence having at least 96% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 97% sequence identity to SEQ ID NO: 30, and the light chain comprises an amino acid sequence having at least 97% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 98% sequence identity to SEQ ID NO: 30, and the light chain comprises an amino acid sequence having at least 98% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 99% sequence identity to SEQ ID NO: 30, and the light chain comprises an amino acid sequence having at least 99% sequence identity to SEQ ID NO: 31. In some embodiments of the present disclosure, an agonistic anti-OX40 monoclonal antibody comprises a heavy chain and a light chain, wherein the heavy chain comprises an amino acid sequence as set forth in SEQ ID NO: 30, and the light chain comprises an amino acid sequence as set forth in SEQ ID NO: 31.

In some embodiments, an anti-PD1 monoclonal antibody comprises a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20. In some embodiments of the present disclosure, an anti-PD1 monoclonal antibody is of human IgG4k isotype.

In some embodiments of the present disclosure, an anti-PD1 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32. In some embodiments of the present disclosure, an anti-PD1 monoclonal antibody comprises a heavy chain, wherein the heavy chain comprises an amino acid sequence having at least 90% sequence identity to SEQ ID NO: 32. In some embodiments of the present disclosure, an anti-PD1 monoclonal antibody comprises a heavy chain, wherein the heavy chain comprises an amino acid sequence having at least 95% sequence identity to SEQ ID NO: 32. In some embodiments of the present disclosure, an anti-PD1 monoclonal antibody comprises a heavy chain, wherein the heavy chain comprises an amino acid sequence having at least 96% sequence identity to SEQ ID NO: 32. In some embodiments of the present disclosure, an anti-PD1 monoclonal antibody comprises a heavy chain, wherein the heavy chain comprises an amino acid sequence having at least 97% sequence identity to SEQ ID NO: 32. In some embodiments of the present disclosure, an anti-PD1 monoclonal antibody comprises a heavy chain, wherein the heavy chain comprises an amino acid sequence having at least 98% sequence identity to SEQ ID NO: 32. In some embodiments of the present disclosure, an anti-PD1 monoclonal antibody comprises a heavy chain, wherein the heavy chain comprises an amino acid sequence having at least 99% sequence identity to SEQ ID NO: 32. In some embodiments of the present disclosure, an anti-PD1 monoclonal antibody comprises a heavy chain, wherein the heavy chain comprises an amino acid sequence as set forth in SEQ ID NO: 32.

In some embodiments of the present disclosure, an anti-PD1 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33. In some embodiments of the present disclosure, an anti-PD1 monoclonal antibody comprises a light chain, wherein the light chain comprises an amino acid sequence having at least 90% sequence identity to SEQ ID NO: 33. In some embodiments of the present disclosure, an anti-PD1 monoclonal antibody comprises a light chain, wherein the light chain comprises an amino acid sequence having at least 95% sequence identity to SEQ ID NO: 33. In some embodiments of the present disclosure, an anti-PD1 monoclonal antibody comprises a light chain, wherein the light chain comprises an amino acid sequence having at least 96% sequence identity to SEQ ID NO: 33. In some embodiments of the present disclosure, an anti-PD1 monoclonal antibody comprises a light chain, wherein the light chain comprises an amino acid sequence having at least 97% sequence identity to SEQ ID NO: 33. In some embodiments of the present disclosure, an anti-PD1 monoclonal antibody comprises a light chain, wherein the light chain comprises an amino acid sequence having at least 98% sequence identity to SEQ ID NO: 33. In some embodiments of the present disclosure, an anti-PD1 monoclonal antibody comprises a light chain, wherein the light chain comprises an amino acid sequence having at least 99% sequence identity to SEQ ID NO: 33. In some embodiments of the present disclosure, an anti-PD1 monoclonal antibody comprises a light chain, wherein the light chain comprises an amino acid sequence as set forth in SEQ ID NO: 33.

In some embodiments of the present disclosure, an anti-PD1 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33. In some embodiments of the present disclosure, an anti-PD1 monoclonal antibody comprises a heavy chain and a light chain, wherein the heavy chain comprises an amino acid sequence having at least 90% sequence identity to SEQ ID NO: 32, and the light chain comprises an amino acid sequence having at least 90% sequence identity to SEQ ID NO: 33. In some embodiments of the present disclosure, an anti-PD1 monoclonal antibody comprises a heavy chain and a light chain, wherein the heavy chain comprises an amino acid sequence having at least 95% sequence identity to SEQ ID NO: 32, and the light chain comprises an amino acid sequence having at least 95% sequence identity to SEQ ID NO: 33. In some embodiments of the present disclosure, an anti-PD1 monoclonal antibody comprises a heavy chain and a light chain, wherein the heavy chain comprises an amino acid sequence having at least 96% sequence identity to SEQ ID NO: 32, and the light chain comprises an amino acid sequence having at least 96% sequence identity to SEQ ID NO: 33. In some embodiments of the present disclosure, an anti-PD1 monoclonal antibody comprises a heavy chain and a light chain, wherein the heavy chain comprises an amino acid sequence having at least 97% sequence identity to SEQ ID NO: 32, and the light chain comprises an amino acid sequence having at least 97% sequence identity to SEQ ID NO: 33. In some embodiments of the present disclosure, an anti-PD1 monoclonal antibody comprises a heavy chain and a light chain, wherein the heavy chain comprises an amino acid sequence having at least 98% sequence identity to SEQ ID NO: 32, and the light chain comprises an amino acid sequence having at least 98% sequence identity to SEQ ID NO: 33. In some embodiments of the present disclosure, an anti-PD1 monoclonal antibody comprises a heavy chain and a light chain, wherein the heavy chain comprises an amino acid sequence having at least 99% sequence identity to SEQ ID NO: 32, and the light chain comprises an amino acid sequence having at least 99% sequence identity to SEQ ID NO: 33. In some embodiments of the present disclosure, an anti-PD1 monoclonal antibody comprises a heavy chain and a light chain, wherein the heavy chain comprises an amino acid sequence as set forth in SEQ ID NO: 32, and the light chain comprises an amino acid sequence as set forth in SEQ ID NO: 33.

In some embodiments, an anti-CTLA4 monoclonal antibody comprises a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments of the present disclosure, an anti-CTLA4 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34. In some embodiments of the present disclosure, an anti-CTLA4 monoclonal antibody comprises a heavy chain, wherein the heavy chain comprises an amino acid sequence having at least 90% sequence identity to SEQ ID NO: 34. In some embodiments of the present disclosure, an anti-CTLA4 monoclonal antibody comprises a heavy chain, wherein the heavy chain comprises an amino acid sequence having at least 95% sequence identity to SEQ ID NO: 34. In some embodiments of the present disclosure, an anti-CTLA4 monoclonal antibody comprises a heavy chain, wherein the heavy chain comprises an amino acid sequence having at least 96% sequence identity to SEQ ID NO: 34. In some embodiments of the present disclosure, an anti-CTLA4 monoclonal antibody comprises a heavy chain, wherein the heavy chain comprises an amino acid sequence having at least 97% sequence identity to SEQ ID NO: 34. In some embodiments of the present disclosure, an anti-CTLA4 monoclonal antibody comprises a heavy chain, wherein the heavy chain comprises an amino acid sequence having at least 98% sequence identity to SEQ ID NO: 34. In some embodiments of the present disclosure, an anti-CTLA4 monoclonal antibody comprises a heavy chain, wherein the heavy chain comprises an amino acid sequence having at least 99% sequence identity to SEQ ID NO: 34. In some embodiments of the present disclosure, an anti-CTLA4 monoclonal antibody comprises a heavy chain, wherein the heavy chain comprises an amino acid sequence as set forth in SEQ ID NO: 34.

In some embodiments of the present disclosure, an anti-CTLA4 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35. In some embodiments of the present disclosure, an anti-CTLA4 monoclonal antibody comprises a light chain, wherein the light chain comprises an amino acid sequence having at least 90% sequence identity to SEQ ID NO: 35. In some embodiments of the present disclosure, an anti-CTLA4 monoclonal antibody comprises a light chain, wherein the light chain comprises an amino acid sequence having at least 95% sequence identity to SEQ ID NO: 35. In some embodiments of the present disclosure, an anti-CTLA4 monoclonal antibody comprises a light chain, wherein the light chain comprises an amino acid sequence having at least 96% sequence identity to SEQ ID NO: 35. In some embodiments of the present disclosure, an anti-CTLA4 monoclonal antibody comprises a light chain, wherein the light chain comprises an amino acid sequence having at least 97% sequence identity to SEQ ID NO: 35. In some embodiments of the present disclosure, an anti-CTLA4 monoclonal antibody comprises a light chain, wherein the light chain comprises an amino acid sequence having at least 98% sequence identity to SEQ ID NO: 35. In some embodiments of the present disclosure, an anti-CTLA4 monoclonal antibody comprises a light chain, wherein the light chain comprises an amino acid sequence having at least 99% sequence identity to SEQ ID NO: 35. In some embodiments of the present disclosure, an anti-CTLA4 monoclonal antibody comprises a light chain, wherein the light chain comprises an amino acid sequence as set forth in SEQ ID NO: 35.

In some embodiments of the present disclosure, an anti-CTLA4 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 90% sequence identity to SEQ ID NO: 34, and the light chain comprises an amino acid sequence having at least 90% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 95% sequence identity to SEQ ID NO: 34, and the light chain comprises an amino acid sequence having at least 95% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 96% sequence identity to SEQ ID NO: 34, and the light chain comprises an amino acid sequence having at least 96% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 97% sequence identity to SEQ ID NO: 34, and the light chain comprises an amino acid sequence having at least 97% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 98% sequence identity to SEQ ID NO: 34, and the light chain comprises an amino acid sequence having at least 98% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 99% sequence identity to SEQ ID NO: 34, and the light chain comprises an amino acid sequence having at least 99% sequence identity to SEQ ID NO: 35. In some embodiments of the present disclosure, an anti-CTLA4 monoclonal antibody comprises a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, a therapy that causes tumor cell lysis and a combination formulation are administered into the same tumor. In some embodiments, a therapy that causes tumor cell lysis and a combination formulation are administered into different tumors.

In some embodiments, a treatment is repeated about every week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks. In some embodiments, a the treatment is repeated about every 3 to 16 weeks, about every 6 to 16 weeks, about every 8 to 16 weeks, about every 10 to 16 weeks, about every 12 to 16 weeks, about every 14 to 16 weeks, about every 3 to 14 weeks, about every 3 to 12 weeks, about every 3 to 10 weeks, about every 3 to 8 weeks, about every 3 to 6 weeks, or about every 3 to 4 weeks. In some embodiments, a treatment is repeated about every 4 to 8 weeks, about every 5 to 8 weeks, about every 6 to 8 weeks, about every 7 to 8 weeks, about every 4 to 7 weeks, about every 4 to 6 weeks, or about every 4 to 5 weeks. In some embodiments, a treatment is repeated about every 4 to 8 weeks.

In some embodiments, a treatment is repeated for 1 to 12 cycles, 1 to 10 cycles, 1 to 8 cycles, 1 to 6 cycles, 1 to 4 cycles, 1 to 2 cycles, 3 to 12 cycles, 5 to 12 cycles, 7 to 12 cycles, 9 to 12 cycles, or 11 to 12 cycles. In some embodiments, a treatment is repeated for 3 to 6 cycles, 3 to cycles, 3 to 4 cycles, 4 to 6 cycles, or 5 to 6 cycles. In some embodiments, a treatment is repeated for 3 to 6 cycles.

In some embodiments, a TLR9 agonist is administered at a dose within the range of about mg to about 10 mg, about 1 mg to about 10 mg, about 2 mg to about 10 mg, about 4 mg to about 10 mg, about 8 mg to about 10 mg, about 0.5 mg to about 8 mg, about 0.5 mg to about 6 mg, about 0.5 mg to about 4 mg, about 0.5 mg to about 2 mg, about 0.5 mg to about 1 mg, about 1 mg to about 4 mg, about 2 mg to about 4 mg, or about 3 mg to about 4 mg.

In some embodiments, an anti-CD40 agonist monoclonal antibody is administered at a dose within the range of about 0.5 mg to about 10 mg, about 1 mg to about 10 mg, about 2 mg to about mg, about 4 mg to about 10 mg, about 8 mg to about 10 mg, about 0.5 mg to about 8 mg, about mg to about 6 mg, about 0.5 mg to about 4 mg, about 0.5 mg to about 2 mg, about 0.5 mg to about 1 mg, about 1 mg to about 8 mg, or about 4 mg to 8 mg.

In some embodiments, an anti-OX40 agonist monoclonal antibody is administered at a dose within the range of about 0.5 mg to about 10 mg, about 1 mg to about 10 mg, about 2 mg to about 10 mg, about 4 mg to about 10 mg, about 8 mg to about 10 mg, about 0.5 mg to about 8 mg, about 0.5 mg to about 6 mg, about 0.5 mg to about 4 mg, about 0.5 mg to about 2 mg, about 0.5 mg to about 1 mg, about 1 mg to about 8 mg, or about 4 mg to 8 mg.

In some embodiments, an anti-PD1 monoclonal antibody is administered at a dose within the range of about 1 mg to about 100 mg, about 2 mg to about 100 mg, about 4 mg to about 100 mg, about 8 mg to about 100 mg, about 10 mg to about 100 mg, about 20 mg to about 100 mg, about 30 mg to about 100 mg, about 40 mg to about 100 mg, about 50 mg to about 100 mg, about 70 mg to about 100 mg, about 90 mg to about 100 mg, about 1 mg to about 90 mg, about 1 mg to about 70 mg, about 1 mg to about 50 mg, about 1 mg to about 40 mg, about 1 mg to about 30 mg, about 1 mg to about 20 mg, about 1 mg to about 10 mg, about 3 mg to about 10 mg, 3 mg to about 30 mg, about 3 mg to about 100 mg, or about 10 mg to about 30 mg.

In some embodiments, an anti-CTLA4 monoclonal antibody is administered at a dose within the range of about 1 mg to about 50 mg, about 2 mg to about 50 mg, about 4 mg to about 50 mg, about 8 mg to about 50 mg, about 10 mg to about 50 mg, about 20 mg to about 50 mg, about 30 mg to about 50 mg, about 40 mg to about 50 mg, about 1 mg to about 40 mg, about 1 mg to about 30 mg, about 1 mg to about 20 mg, about 1 mg to about 10 mg, about 1 mg to about 5 mg, about 1 mg to about 2 mg, about 5 mg to about 15 mg, about 5 mg to about 40 mg, or about 15 mg to about 40 mg.

In some embodiments, a TLR9 agonist, an anti-CD40 agonistic antibody, an anti-OX40 agonistic antibody, and an anti-CTLA4 antibody are administered at a dose of about 1 mg, about 1 mg, about 1 mg, and about 1 mg, respectively.

In some embodiments, a TLR9 agonist, an anti-CD40 agonistic antibody, an anti-OX40 agonistic antibody, and an anti-CTLA4 antibody are administered at a dose of about 2 mg, about 5 mg, about 5 mg, and about 5 mg, respectively.

In some embodiments, a TLR9 agonist, an anti-CD40 agonistic antibody, an anti-OX40 agonistic antibody, and an anti-CTLA4 antibody are administered at a dose of about 3 mg, about 7.5 mg, about 7.5 mg, and about 15 mg, respectively.

In some embodiments, a TLR9 agonist, an anti-CD40 agonistic antibody, an anti-OX40 agonistic antibody, and an anti-CTLA4 antibody are administered at a dose of about 4 mg, about 10 mg, about 10 mg, and about 40 mg, respectively.

In some embodiments, a TLR9 agonist, an anti-CD40 agonistic antibody, an anti-PD1 antibody, and an anti-CTLA4 antibody are administered at a dose of about 1 mg, about 1 mg, about 3 mg, and about 1 mg, respectively.

In some embodiments, a TLR9 agonist, an anti-CD40 agonistic antibody, an anti-PD1 antibody, and an anti-CTLA4 antibody are administered at a dose of about 2 mg, about 5 mg, about 10 mg, and about 5 mg, respectively.

In some embodiments, a TLR9 agonist, an anti-CD40 agonistic antibody, an anti-PD1 antibody, and an anti-CTLA4 antibody are administered at a dose of about 3 mg, about 7.5 mg, about 30 mg, and about 15 mg, respectively.

In some embodiments, a TLR9 agonist, an anti-CD40 agonistic antibody, an anti-PD1 antibody, and an anti-CTLA4 antibody are administered at a dose of about 4 mg, about 10 mg, about 80 mg, and about 40 mg, respectively.

In some embodiments, a TLR9 agonist, an anti-CD40 agonistic antibody, an anti-PD1 antibody, and an anti-CTLA4 antibody are administered at a dose of about 4 mg, about 10 mg, about 100 mg, and about 40 mg, respectively.

In some embodiments, a combination formulation is administered in a total volume within the range of about 1 mL to about 30 mL, about 1 mL to about 25 mL, about 1 mL to about 20 mL, about 1 mL to about 15 mL, about 1 mL to about 10 mL, about mL to about 5 mL, about 1 mL to about 2 mL, about 2 mL to about 30 mL, about 5 mL to about 30 mL, about 10 mL to about 30 mL, about 15 mL to about 30 mL, about 20 mL to about 30 mL, about 25 mL to about 30 mL, about 5 mL to about 25 mL, about 10 mL to about 20 mL, about 10 mL to about 15 mL, or about 15 mL to about 20 mL. In some embodiments, a combination formulation is administered in a total volume within the range of about 1 mL to about 30 mL. In some embodiments, a total volume is about 15 mL.

In some embodiments, a cancer is a solid tumor cancer. In some embodiments, a solid tumor cancer is selected from adenocarcinoma, astrocytoma, bladder cancer, bone sarcoma, breast cancer, cervical cancer, chordoma, colorectal cancer, endometrial cancer, esophageal cancer, glioblastoma, glioma, kidney cancer, liver cancer, medulloblastoma, melanoma, meningioma, mesothelioma, metastatic pituitary carcinoma, prostate cancer, neuroblastoma, non-melanoma skin cancer, non-small cell lung cancer, oral cancer, ovarian cancer, pancreatic cancer, renal cell carcinoma, retinoblastoma, sarcoma, small cell lung cancer, squamous cell carcinoma (including head and neck cancer), stomach cancer, testicular cancer, thyroid cancer, and Wilms tumor. In some embodiments, a cancer is metastatic prostate cancer. In some embodiments, a cancer is metastatic breast cancer. In some embodiments, a cancer is non-small cell lung cancer.

The present disclosure provides pharmaceutical compositions comprising: a. a TLR9 agonist; b. an agonistic anti-CD40 monoclonal antibody; c. an anti-CTLA4 monoclonal antibody; d. an agonistic anti-OX40 monoclonal antibody; and e. pharmaceutical acceptable excipients. The present disclosure further provides pharmaceutical compositions consisting of: a. a TLR9 agonist; b. an agonistic anti-CD40 monoclonal antibody; c. an anti-CTLA4 monoclonal antibody; d. an agonistic anti-OX40 monoclonal antibody; and e. pharmaceutical acceptable excipients.

In some embodiments, a TLR9 agonist is a nucleic acid as set forth in SEQ ID NO: 1, SEQ ID NO: 2, or SEQ ID NO: 27. In some embodiments, an agonistic anti-CD40 monoclonal antibody comprises a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8. In some embodiments, an agonistic anti-OX40 monoclonal antibody comprises a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14. In some embodiments, an anti-CTLA4 monoclonal antibody comprises a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a TLR9 agonist is a nucleic acid as set forth in SEQ ID NO: 1, SEQ ID NO: 2, or SEQ ID NO: 27. In some embodiments, an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments, an agonistic anti-OX40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31. In some embodiments, an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, a TLR9 agonist is a nucleic acid as set forth in SEQ ID NO: 1, SEQ ID NO: 2, or SEQ ID NO: 3. In some embodiments, an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29. In some embodiments, an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31. In some embodiments, an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, respective doses of TLR9 agonist, anti-CD40 agonistic antibody, anti-OX40 agonistic antibody, and anti-CTLA4 antibody are selected from: a. about 1 mg, about 1 mg, about 1 mg, and about 1 mg, respectively; b. about 2 mg, about 5 mg, about 5 mg, and about 5 mg, respectively; c. about 3 mg, about 7.5 mg, about 7.5 mg, and about 15 mg, respectively; and d. about 4 mg, about 10 mg, about 10 mg, and about 40 mg, respectively.

The present disclosure provides pharmaceutical compositions comprising: a. a TLR9 agonist; b. an agonistic anti-CD40 monoclonal antibody; c. an anti-CTLA4 monoclonal antibody; d. an agonistic anti-PD1 monoclonal antibody; and e. pharmaceutical acceptable excipients. The present disclosure further provides pharmaceutical compositions consisting of: a. a TLR9 agonist; b. an agonistic anti-CD40 monoclonal antibody; c. an anti-CTLA4 monoclonal antibody; d. an agonistic anti-PD1 monoclonal antibody; and e. pharmaceutical acceptable excipients.

In some embodiments, a TLR9 agonist is a nucleic acid as set forth in SEQ ID NO: 1, SEQ ID NO: 2, or SEQ ID NO: 27. In some embodiments, an agonistic anti-CD40 monoclonal antibody comprises a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8. In some embodiments, an anti-PD1 monoclonal antibody comprises a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20. In some embodiments, an anti-CTLA4 monoclonal antibody comprises a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, a TLR9 agonist is a nucleic acid as set forth in SEQ ID NO: 1, SEQ ID NO: 2, or SEQ ID NO: 27. In some embodiments, an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments, an anti-PD1 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33. In some embodiments, an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, a TLR9 agonist is a nucleic acid as set forth in SEQ ID NO: 1, SEQ ID NO: 2, or SEQ ID NO: 27. In some embodiments, an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29. In some embodiments, an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33. In some embodiments, an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, respective doses of TLR9 agonist, anti-CD40 agonistic antibody, anti-PD1 antibody and anti-CTLA4 antibody are selected from: a. about 1 mg, about 1 mg, about 3 mg, and about 1 mg, respectively; b. about 2 mg, about 5 mg, about 10 mg, and about 5 mg, respectively; c. about 3 mg, about 7.5 mg, about 30 mg, and about 15 mg, respectively; and d. about 4 mg, about 10 mg, about 100 mg, and about 40 mg, respectively.

In some embodiments, respective doses of TLR9 agonist, anti-CD40 agonistic antibody, anti-PD1 antibody and anti-CTLA4 antibody are selected from: a. about 1 mg, about 1 mg, about 3 mg, and about 1 mg, respectively; b. about 2 mg, about 5 mg, about 10 mg, and about 5 mg, respectively; c. about 3 mg, about 7.5 mg, about 30 mg, and about 15 mg, respectively; d. about 4 mg, about 10 mg, about 80 mg, and about 40 mg, respectively, and e. about 4 mg, about 10 mg, about 100 mg, and about 40 mg, respectively.

The present disclosure provides, among other things, methods of treating cancer in a subject, the methods comprising steps of: a) administering to a tumor in the subject a cryolysis therapy that causes partial tumor cell lysis; and b) administering to the same tumor in step a) a therapeutically effective amount of a combination formulation, wherein the combination formulation comprises: a. a TLR9 agonist; b. an agonistic anti-CD40 monoclonal antibody; c. an anti-CTLA4 monoclonal antibody; and d. an agonistic anti-OX40 monoclonal antibody; wherein the cryolysis therapy is administered prior to administration of the combination formulation.

The present disclosure provides, among other things, methods of treating cancer in a subject, the methods comprising steps of: a) administering to a tumor in the subject a cryolysis therapy that causes partial tumor cell lysis; and b) administering to the same tumor in step a) a therapeutically effective amount of a combination formulation, wherein the combination formulation comprises: a. a TLR9 agonist; b. an agonistic anti-CD40 monoclonal antibody; c. an anti-CTLA4 monoclonal antibody; and d. an anti-PD1 monoclonal antibody; wherein the cryolysis therapy is administered prior to administration of the combination formulation.

In some embodiments, a combination formulation is any pharmaceutical composition as described herein.

### BRIEF DESCRIPTION OF THE DRAWING

FIG. 1 shows a representation of a mechanism of action of CpG oligodeoxynucleotides as TLR9 agonist on human B cells and plasmacytoid dendritic cells.

FIG. 2 shows a representation of a mechanism of action of agonistic anti-CD40 monoclonal antibodies on B cells, dendritic cells and macrophages.

FIG. 3 shows a representation of a mechanism of action of blocking anti-CTLA4 monoclonal antibodies

FIG. 4 shows a representation of a mechanism of action of agonistic anti-OX40 monoclonal antibodies

FIG. 5 shows a representation of a mechanism of action of blocking anti-PD1 monoclonal antibodies

FIG. 6 shows a representation of three immunological states of a tumor and schematics of intratumoral partial cryolysis and formation of an ice ball that is smaller than the tumor.

FIG. 7 shows a representation of schematics of intratumoral injection and infusion of therapeutic components of SV-101 into a region previously treated by partial cryolysis.

FIG. 8 shows a representation of schematics of intratumoral injection and infusion of therapeutic components of SV-102 into a region previously treated by partial cryolysis.

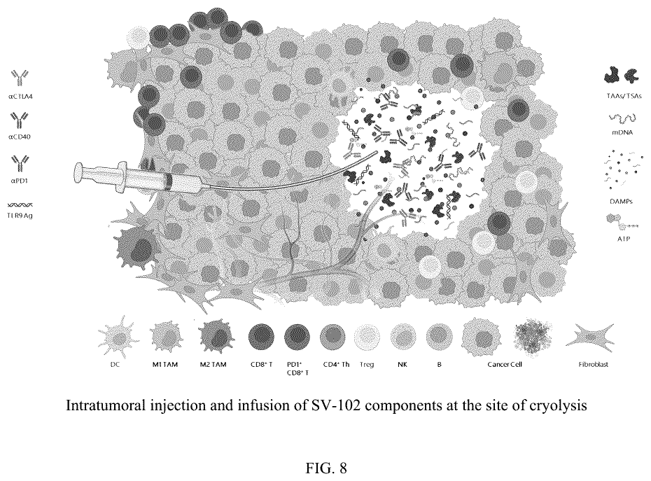

FIG. 9 shows a representation of a proposed mechanism of action for synergistic activation of macrophages mediated by concomitant treatment of anti-CD40 agonistic mAbs and TLR9 agonists.

FIG. 10 shows a representation of a proposed mechanism of action for synergistic activation of B cells mediated by concomitant treatment of anti-CD40 agonistic mAbs and TLR9 agonists.

FIG. 11 shows a representation of a proposed mechanism of action for synergistic activation of dendritic cells (e.g., pDCs and cDCs) mediated by concomitant treatment of anti-CD40 agonistic mAbs and TLR9 agonists.

FIG. 12 shows a representation of a proposed mechanism of action for synergistic activation CD4+ and CD8+ T cells by dendritic cells in TDLNs, TME and TLS, as well as suppression of Treg function, mediated by combined treatment with anti-CTLA4 mAb, anti-CD40 agonistic mAb and anti-OX40 agonistic mAb.

FIG. 13 shows a representation of a proposed mechanism of action for synergistic activation CD4+ and CD8+ T cells by dendritic cells in TDLNs, TME and TLS, as well as suppression of Treg function, mediated by combined treatment with anti-CTLA4 mAb, anti-CD40 agonistic mAb and anti-PD1 mAb.

FIG. 14 shows a representation of a proposed mechanism of action in TME for synergistic suppression of Treg function, mediated by combination of anti-OX40 mAb and anti-CTLA4 mAb.

FIG. 15 shows a representation of a proposed mechanism of action in TME for synergistic reactivation of Teff cells by combined action of anti-CD40 agonistic mAb and anti-PD1 mAb.

FIG. 16 shows a representation of a proposed mechanism of action in TME for synergistic suppression of Treg function and reactivation of exhausted Teff cells, mediated by combination of anti-PD1 mAb and anti-CTLA4 mAb.

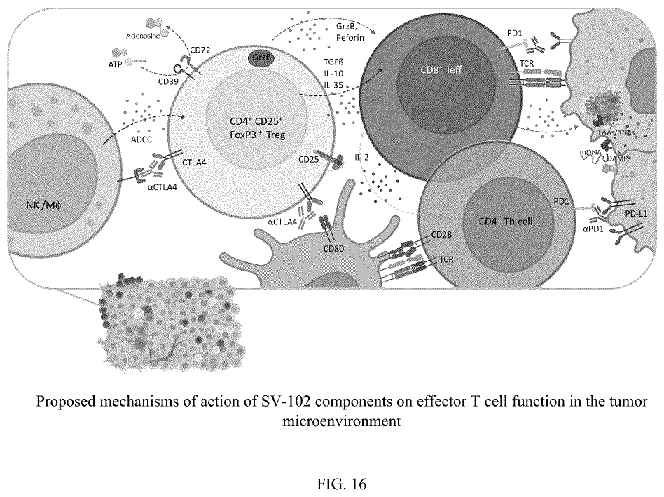

FIG. 17 shows a representation of isotherms around a cryoprobe during cryolysis. The temperature within the represented cryolysis region is −40° C. to −60° C.

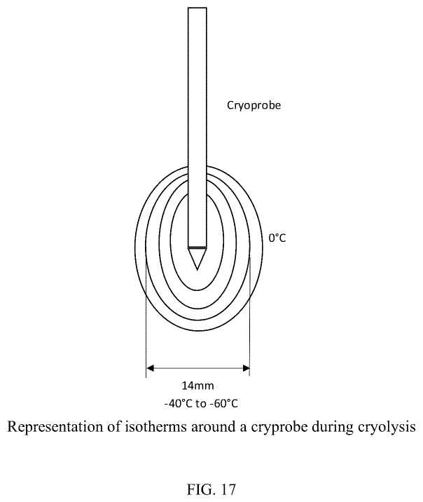

FIG. 18 shows multiparametric MRI with diffusion weighted sequences shows no evidence of residual tumor in the prostate (panels A-C).

FIG. 19 shows coronal images of lung windows of CT scans done at days 1, 28 and 56 post-treatment. The right sided pleural effusion has resolved to barely detectable, and the intraparenchymal lung lesions have shrunk by 40% in the combined diameters, indicating a partial response.

### DETAILED DESCRIPTION

### Definitions

Administration: As used herein, the term “administration” refers to the administration of a therapy (e.g., a composition/formulation or other form of treatment such as cryotherapy) to a subject or system. Administration to an animal subject (e.g., to a human) may be by any appropriate route. For example, in some embodiments, administration may be intratumoral. In some embodiments, administration is conducted by infusion or perfusion (e.g., using methods described herein). In some embodiments, administration is an intratumoral administration that is aimed at a loco-regional therapy where an injected formulation is not expected to be fully contained within a tumor tissue but is actually expected to enter into lymphoid structures and peritumoral tissue connected to the tumor via lymphatic vessels.

Agent: The term “agent” as used herein may refer to a compound or entity of any chemical class including, for example, polypeptides, nucleic acids, saccharides, lipids, small molecules, metals, or combinations thereof. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. Some particular embodiments of agents that may be utilized in accordance with the present disclosure include antibodies, antibody fragments, nucleic acids. In some particular embodiments, an agent of the present disclosure is a CpG oligodeoxynucleotide (CpG ODN). In some particular embodiments, an agent of the present disclosure is a monoclonal antibody. In some aspects of the present disclosure, the agent is a “therapeutic agent” or a “pharmacologic agent” that elicits a desired pharmacological effect when administered to an organism (e.g., a subject). In some embodiments, said agent that elicits a desired pharmacological effect when administered to an organism can be said to be “active” or “pharmacologically active.” In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, an agent is an ingredient in a combination formulation or composition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Antibody: As used herein, the term “antibody” as used to herein may include whole antibodies and any antigen-binding fragments (i.e., “antigen-binding portions”) or single chains thereof. An “antibody” refers, in some embodiments, to a glycoprotein comprising at least two heavy chains and two light chains inter-connected by disulfide bonds, or an antigen binding fragment thereof. Each heavy chain is comprised of a heavy chain variable region (abbreviated as VH) and a heavy chain constant region. In certain naturally occurring IgG, IgD and IgA antibodies, a heavy chain constant region is comprised of three domains, CH1, CH2 and CH3. In certain naturally occurring antibodies, each light chain is comprised of a light chain variable region (abbreviated as VL) and a light chain constant region. In some embodiments, a light chain constant region is comprised of one domain, CL. VH and VL regions can be further subdivided into regions of hypervariability, termed complementarity determining regions (CDR), interspersed with regions that are more conserved, termed framework regions (FR). Each VH and VL is composed of three CDRs and four framework regions (FRs), arranged from amino-terminus to carboxy-terminus in the following order: FR1, CDR1, FR2, CDR2, FR3, CDR3, FR4. Variable regions of a heavy chain and/or a light chains contain a binding domain that interacts with an antigen. In some embodiments, constant regions of antibodies may mediate the binding of the immunoglobulin to host tissues or factors, including various cells of the immune system (e.g., effector cells) and the first component (C1q) of the classical complement system. Unless otherwise indicated, an antibody may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids. Unless otherwise indicated, “antibody” may include, by way of example, monoclonal and polyclonal antibodies; chimeric and humanized antibodies; human and non-human antibodies; wholly synthetic antibodies; and single chain antibodies.

Combination therapy: As used herein, the term “combination therapy” may refer to those situations in which a subject is simultaneously exposed to two or more therapeutic regimens or therapies (e.g., with two or more therapeutic agents). In some embodiments, two or more agents may be administered simultaneously (i.e., co-administration); in some embodiments, such agents may be administered sequentially (in a particular order, or in any order, as indicated by the context); in some embodiments, such agents are administered in overlapping dosing regimens. In some embodiments, a combination therapy is administered in the form of a combination formulation or composition (e.g., combination formulations and compositions described herein). In some embodiments, multiple agents of a composition or formulation are mixed just prior to administration.

Cryoablation: As used herein, the term “cryoablation” may refer to a process that uses localized, extreme cold to destroy a target tissue, e.g., an abnormal or diseased tissue, such as a tumor. Cryoablation is performed using hollow needles (e.g., cryoprobes) through which cooled, thermally conductive, fluids are circulated. During cryoablation, cryoprobes are positioned adjacent to, or within (e.g., intratumoral positioning), a target tissue in such a way that a freezing process will destroy abnormal or diseased tissue, such as a tumor, completely or to a degree that is substantially complete. Once a cryoprobe is in place, an attached cryogenic freezing unit removes heat from (“cools”) the tip of the cryoprobe and by extension from surrounding tissues in a target zone. Cryoablation occurs in tissue that has been frozen by at least three mechanisms: 1. formation of ice crystals within cells thereby disrupting membranes, and interrupting cellular metabolism among other processes; 2. coagulation of blood thereby interrupting blood flow to a tissue in turn causing ischemia and cell death; and 3. induction of apoptosis, the so-called programmed cell death cascade. Cryoablation is amenable for treatment of most solid tumors. A skilled medical practitioner will understand which tumors are amenable for treatment by cryotherapies such as cryoablation, or cryolysis (e.g., cryolysis therapy as described herein). In many embodiments of the present disclosure, administration of cryotherapy is conducted in such a manner as to avoid cryoablation. In some embodiments of the present disclosure, cryotherapy is conducted in such a manner as to promote cryolysis (i.e., partial lysis of tumor tissue) as opposed to cryoablation.

Cryolysis: As used herein, the term “cryolysis” may refer to a process that uses localized, extreme cold to partially destroy a target tissue, e.g., an abnormal or diseased tissue, such as a tumor. Thus, cryolysis, as performed in methods described herein, is similar to cryoablation, but cryolysis does not aim to ablate an entire tumor. Accordingly, during administration of a therapy that causes cell lysis by cryolysis, cryoprobes are positioned adjacent to, or within (e.g., intratumoral positioning), a target tissue in such a way that a freezing process will partially destroy abnormal or diseased tissue, such as a tumor. In many embodiments of the present disclosure, a goal of cryolysis is not to achieve full ablation of the tumor tissue (i.e., cryoablation), but to elicit a partial necrotic zone within a tumor to release sufficient amounts of TSA/TAA and DAMPs to initiate an immune response. Accordingly, cryolysis may differ from cryoablation in methods of administration including, but not limited to, minimum temperature achieved in a target tissue treatment zone, rate of cooling of a target tissue treatment zone (which is related to, among other things, rate of cooling of a cryotherapy device, such as a cryoprobe), diameter of an ice ball formed at a target tissue treatment zone, diameter of an isotherm within an ice ball that reaches a critical temperature for necrosis and apoptosis, duty cycle of a cryotherapy device, speed of thaw, and duration of freeze-thaw cycles (Cooperberg, M. and Kim D. K. (2020); Littrup, P. J., et al. (2009); Shah, T. T., et al. (2016)).

Cryoprobe: As used herein, the term “cryoprobe” may refer to a hollow needle through which a cooled, thermally conductive, fluid is circulated. A thermally conductive fluid may be any acceptable cryogen used in the field, for example, argon, nitrous oxide, carbon dioxide, or liquid nitrogen. A cryoprobe may be of any diameter that is commonly used in the field for intratumoral cryotherapy. In some embodiments, a cryoprobe has a diameter of about 1.5 mm to about 3.4 mm. In some embodiments, a cryoprobe has a diameter of about 1.5 mm. In some embodiments, a cryoprobe has a diameter of about 3.4 mm. In some embodiments, a cryoprobe diameter is selected to enable a rapid cooling rate. In some embodiments, achieving a rapid cooling rate requires that a cooling probe achieves a temperature of about −40° C. or colder, about −50° C. or colder, or about −60° C. or colder, in less than a minute. In some embodiments, a cryoprobe surface may reach −130° C. to −150° C. and as low as −190° C. as need for rapid cooling of target tissue, however, it is generally accepted that such low temperatures are not required for cell death (Littrup, P. J., et al. (2009); Shah, T. T., et al. (2016)). In some embodiments, cryoprobe diameter is selected to enable efficient cryolysis of a target tissue, e.g., tumor cells, which is to say that the selected cryoprobe effectively elicits a partial necrotic zone within a tumor to release sufficient amounts of TSA and DAMPs to initiate an immune response.

Cryotherapy: As used herein, the terms “cryotherapy” or “cryosurgery” may refer to controlled, localized use of freezing temperatures in medical therapy. Cryotherapy may be used to treat a variety of tissue lesions. For example, in cancer treatment, cryosurgery is an application of extremely low temperatures to destroy abnormal or diseased tissue (e.g., tumor tissue) either completely (i.e., cryoablation) or partially (i.e., cryolysis, or partial cryolysis). In some embodiments, administration of a cryotherapy treatment causes damage to a treated tissue due to intracellular ice formation. The degree of damage of a target tissue depends upon many factors including, but not limited to, minimum temperature achieved in a target tissue treatment zone, rate of cooling of a target tissue treatment zone (which is related to, among other things, rate of cooling of a cryotherapy device, such as a cryoprobe), diameter of an ice ball formed at a target tissue treatment zone, diameter of an isotherm within an ice ball that reaches a critical temperature for necrosis and/or apoptosis, speed of thaw, duty cycle of a cryotherapy device, and duration of freeze-thaw cycles (Cooperberg, M. and Kim D. K. (2020); Littrup, P. J., et al. (2009); Shah, T. T., et al. (2016)).

Duty cycle: As used herein, the term “duty cycle” may refer to a fraction of one period in which a signal, system, or device is active. A period is the time it takes for a signal, system, or device to complete an on-and-off cycle, or a cycle between two states. Duty cycle is commonly expressed as a percentage or a ratio. In some embodiments of the present disclosure, duty cycle describes the percentage of time a cryogen is flowing, or circulating, within a cryoprobe. In some embodiments, a duty cycle of about 50%, about 60%, about 70%, about 80%, about 90%, or about 100% is used. In some embodiments, a duty cycle of about 100% is used. In some embodiments of the present disclosure, a duty cycle is selected to provide a rapid freeze rate of a tumor cell where a zone of tumor cells reaches about −40° C. or colder, about −50° C. or colder, or about −60° C. or colder in less than 2 minutes, less than 1.75 minutes, less than 1.5 minutes, less than 1.25 minutes, less than 1 minute, less than 50 seconds, less than 40 seconds, less than 30 seconds, less than 20 seconds, or less than 10 seconds. In some embodiments of the present disclosure, a duty cycle is selected to provide a rapid freeze rate of a tumor cell where a zone of tumor cells reaches about −40° C. or colder, about −50° C. or colder, or about −60° C. or colder in less than one minute.

Subject: As used herein, the terms “subject” or “patient” refer to any animal, including a mammal, to which a provided method and/or composition is or may be administered, e.g., for experimental, diagnostic, prophylactic, and/or therapeutic purposes. The term “mammal” as used herein, encompasses any mammal. Examples of mammals include, but are not limited to, cows, horses, sheep, pigs, cats, dogs, mice, rats, rabbits, guinea pigs, monkeys, humans, etc. The subject is preferably a human. In some embodiments a subject is suffering from or is susceptible to one or more disorders or conditions. In some embodiments, a subject displays one or more symptoms of a disorder or condition. In some embodiments, a subject has been diagnosed with one or more disorders or conditions. In some embodiments, a disorder or condition is or includes a proliferative disease such as cancer. In some embodiments, a cancer is a solid tumor cancer. In some embodiments, a subject is receiving or has received certain therapy to diagnose and/or to treat a disease, disorder, or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena, including those related to therapeutic treatment.

Treatment: As used herein, the term “treatment” (also “treat” or “treating”) refers to any administration of a composition or formulation (e.g., those described herein) or provided method that partially or completely alleviates, ameliorates, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. In some embodiments, refers to an amelioration, prophylaxis, or reversal of a disease or disorder, or at least one discernible symptom thereof. For example, treating cancer, in particular a solid tumor cancer. A treatment may be administered in cycles, or treatment cycles. In some embodiments, a treatment cycle involves administration of a treatment regimen (e.g., methods set forth in the present disclosure) to a subject followed by a period of rest to allow the subject to recover. In some embodiments, a period of rest after a subject receives treatment is about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, or about 16 weeks. In some embodiments, a treatment is repeated for 1 to 12 cycles, 1 to 10 cycles, 1 to 8 cycles, 1 to 6 cycles, 1 to 4 cycles, 1 to 2 cycles, 3 to 12 cycles, 5 to 12 cycles, 7 to 12 cycles, 9 to 12 cycles, or 11 to 12 cycles. In some embodiments, a treatment is repeated for 3 to 6 cycles, 3 to 5 cycles, 3 to 4 cycles, 4 to 6 cycles, or 5 to 6 cycles. In some embodiments, a treatment is repeated for 3 to 6 cycles.

Therapeutically effective amount: As used herein, a “therapeutically effective amount” of an agent, or composition/formulation, of the present disclosure (e.g., an antibody, oligonucleotide, etc.) means the amount of the agent, or compositions/formulation, that is effective to treat or prevent recurrence of a condition or its signs or symptoms. The full therapeutic effect does not necessarily occur by administration of one dose, and may occur only after administration of a series of doses. Thus, a therapeutically effective amount may be administered in one or more administrations of a unit dosage form.

Pharmaceutically acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to a recipient thereof.

### DETAILED DESCRIPTION

The present disclosure appreciates that given the complex and multifactorial nature of the mechanisms mediating tumor immune evasion, it is becoming increasingly clear that cancer immunotherapy needs to implement a combination of many therapeutic agents that elicit multiple immuno-pharmacologic effects (Melero, Berman et al. 2015, Vilgelm, Johnson et al. 2016).

The present disclosure further appreciates that a comprehensive antitumor immunotherapeutic approach ideally needs to: a) elicit immunogenic tumor cell death, characterized by the necrotic death and release of TAA/TSA and danger associated molecular patterns (DAMPs); b) recruit and activate innate immune cells (e.g., NK, macrophages, etc.) and antigen presenting cells (APCs) that take up, process and present the TSA/TAAs-derived peptides to mediate the priming of an adaptive immune response to such antigens; c) stimulate the activation and proliferation of effector T cells while avoiding counterregulatory mechanisms that limit T cell priming; and d) block pre-existing and inducible immunosuppressive mechanisms, by reverting mechanism mediating T cell exhaustion, by blocking the function of or depleting regulatory T cells (Tregs), and by preventing recruitment of myeloid derived suppressor cells (MDSCs) to the tumor microenvironment (Chen and Mellman 2013).

The present disclosure recognizes that there are multiple therapeutic approaches that boost immunogenic tumor cell death and TAA/TSA release (Green, Ferguson et al. 2009, Dudek, Garg et al. 2013, Zhou, Wang et al. 2019). These include radiotherapy, chemotherapy, oncolytic viruses, toxins, lytic peptides, lytic compounds, cryoablation, electroporation, radiofrequency ablation, antibody-drug conjugates, and others.

Additionally, there are different approaches being investigated to recruit, activate and promote the immunogenic maturation of APCs to a tumor bed, and these involve the use of cytokines (e.g., GM-CSF, FLT-3 ligand), chemokines (e.g., CCL21, CXCL10, etc.), TLRs ligands (e.g. CpG ODNs), STING agonists and agonistic anti-CD40 (αCD40) ligands. Additionally, vaccination strategies based on the dosing of peptide/adjuvants, DNA or RNA vaccines encoding single or personalized TSAs aim at providing tumor antigens in a context that favors antigen uptake and presentation by professional APCs with the goal of stimulating a strong adaptive immune response. Similarly, there are several investigational strategies that promote the activation and proliferation of NK cells and effector CD4 helper and CD8 cytotoxic T cells. Those studies include the use of interleukins (e.g., IL-2, IL-12, IL-15, etc.), and agonistic ligands of the TNFR family such as OX40, 4-1BB, GITR, ICOS, CD27 and others (Choi, Shi et al. 2020).

The present disclosure further recognizes that one of the most effective immunotherapeutic approaches in the clinical setting has been to block immunosuppressive mechanisms mediated by Tregs, which affect the function of helper and effector T cells as well as that of dendritic cells (DCs). The main checkpoint inhibitor expressed by Tregs is CTLA-4, and its blockade with antagonistic anti-CTLA4 (αCTLA) mAbs has represented a breakthrough in the immunotherapy of cancer (Wei, Duffy et al. 2018). Additionally, effector T cells express numerous immune checkpoints that mediate exhaustion and anergy, such as PD-1, TIM-3, TIGIT, LAG-3, VISTA and BTLA, and their blockade with antagonistic mAbs is being actively investigated (Melero, Berman et al. 2015). The most advanced of these approaches is the blockade of PD-1 checkpoint (or its ligand PD-L1 expressed by tumor cells or immunosuppressive DCs), which reinvigorates exhausted T cells and today constitutes a central backbone of almost every immunotherapeutic approach. However, the combined use of CTLA-4 and PD-1/PD-L1 blockade relies on pre-existing antitumor immunity and tumor infiltrating T cells and still fails to completely address the mechanisms of enhanced tumor lysis, dendritic cell activation, promotion of antigen presentation, activation of naïve T cells, and direct stimulation of effector T cell proliferation. For these reasons, antitumor response using this combination is observed more frequently only in certain “hot” immunogenic cancers (e.g., melanoma, lung cancer) and less frequently in “cold” non-immunogenic cancers (e.g., breast, prostate, pancreatic cancer, etc.) (Das, Verma et al. 2015, Winograd, Byrne et al. 2015, Vilgelm, Johnson et al. 2016, Chae, Arya et al. 2018, Chowdhury, Chamoto et al. 2018).

Therefore, there is a strong rationale for combining therapeutic approaches that target different stages and functions of an antitumor immune response, synchronizing tumor antigen release with APC recruitment and activation, promotion of activation and proliferation of effector T cells, with concomitant suppression of pre-existing and inducible counterregulatory immunosuppressive mechanisms mediated by Tregs, MDSCs and the tumor itself. However, despite the obvious therapeutic potential of combining therapeutic approaches, there are several barriers to the development of immunotherapies involving the use of combinations of investigational agents, and accordingly, several anticipated combinations have not yielded expected outcomes, e.g., increased antitumor responses. One such barrier arises due to agents often being developed individually or in combination with only one or two other agents. The suboptimal activity of individual or a few agents is often compensated by resorting to the use of high doses, which maximizes their potential serious adverse effects (AEs). Another barrier arises due to most of investigational agents being administered systemically, which requires higher doses than what is actually needed at sites of action (e.g., tumor, tumor draining lymph nodes (TDLNs) and peritumoral tertiary lymphoid structures), thus increasing systemic exposure and risk of off-target toxicities. Moreover, any added efficacy benefit of using a particular combination of agents is often counterbalanced by additive serious AEs, such as cytokine storm, ulcerative colitis, hepatitis, thyroiditis, hypophysitis and other immune-related AEs that often force the discontinuation of these therapies (Xu, Xu et al. 2020, Biniakewitz, Kasler et al. 2021).

A potential solution to the use of high systemic doses of multiple immunotherapeutic agents has been to dose some of those agents by intratumoral or peritumoral injection at lower doses, which has shown good efficacy and reduced AEs in preclinical models (Marabelle, Kohrt et al. 2014, Aznar, Tinari et al. 2017). However, these approaches still rely on the dosing of only one or two intratumoral agent with the others administered systemically at high doses.

Therefore, current immunotherapeutic approaches relying on the use of one or two agents are only partially addressing some of those immunologic mechanisms, thus still fail to provide a comprehensive solution to the problem of tumor-mediated immune evasion.

The methods and compositions of the present disclosure provide a solution to these problems, maximizing antitumor efficacy by synchronized and simultaneous targeting of multiple immunologic mechanisms, optimizing the biodistribution of active pharmacological ingredients while minimizing the likelihood of serious adverse events. In some embodiments, the methods and compositions of the present disclosure stimulate autologous vaccination against unique TSA/TAAs by implementing a strategy that combines induction of immunogenic tumor cell death by physical and/or pharmacologic methods with an intratumoral poly-pharmacologic immune therapeutic strategy that mediates APC recruitment and activation, promotion of activation and expansion of effector T cells, with concomitant suppression of pre-existing and inducible counterregulatory immunosuppressive mechanisms mediated by Tregs and cells at the tumor microenvironment. In some embodiments, the methods of the present disclosure aim at generating a systemic immune response against a tumor upon local immune stimulation by using said tumor as its own vaccine, thus generating a polyclonal adaptive immune response mediated by T and/or B-cells against pre-existing TSA/TAAs. An advantage of the methods and compositions of the present disclosure over regular cancer vaccines is that, in some embodiments, they use off-the-shelf immune stimulatory products, do not require pre-treatment, molecular target identification of tumor antigens, nor HLA-type patient identification and selection, resulting in a more of a universal therapeutic strategy compared to a personalized cancer vaccine approach, which is usually directed against specific tumor antigens that must be shared between a vaccine and a tumor.

The methods and compositions provided herein mediate multiple pharmacologic effects aimed at mounting a synchronized and multi-faceted antitumor immune response.

Administration of Tumor Cell Lysis Therapies

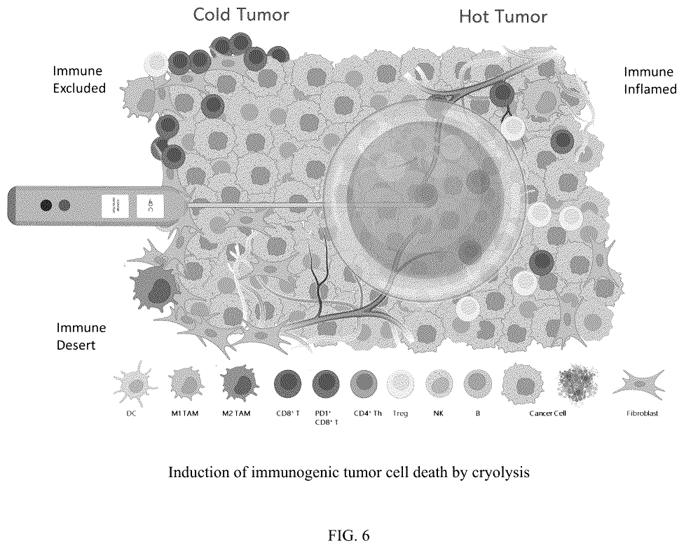

The methods of the present disclosure, in some embodiments, comprise a first step aimed at initiating in situ immunization by triggering cell lysis and immunogenic cell death of tumor cells, using an intratumoral cryolysis device and a second step comprising the intratumoral administration of a combination of immunotherapeutic agents that mediate different pharmacologic effects (FIG. 6).

In some embodiments, methods of the present disclosure comprise a first step of triggering cell lysis and immunogenic cell death of tumor cells (e.g., by intratumoral cryolysis), which mediates necrotic cell death followed by the release of TSA/TAAs and DAMPs into a tumor microenvironment. One goal of a cryolysis step, as performed in some embodiments of the present disclosure, is not to achieve full ablation of the tumor tissue (i.e., cryoablation) but to elicit a partial necrotic zone (as a result of a partial cryolysis) within the tumor to release sufficient amounts of TSA and DAMPs to initiate an immune response (den Brok, Sutmuller et al. 2004, Sidana 2014). In some embodiments of the present disclosure a partial cryolysis resulting from 1-2 freeze-thaw cycles is preferred to preserve vascular and lymphatic structures that facilitate migration of antigen, cytokines, therapeutic agents, and immune cells between a TDLN and a tumor microenvironment. In some embodiments, a number of freeze-thaw cycles is selected to minimize damage to vascular and lymphatic structures and/or immunosuppressive mechanisms. In some embodiments, one freeze-thaw cycle is preferred. In some embodiments, two freeze-thaw cycles are preferred. In some embodiments, no more than one freeze-thaw cycle is preferred. In some embodiments, no more than two freeze-thaw cycles are preferred. In some embodiments, no more than three freeze-thaw cycles are preferred. In some embodiments, a step of triggering cell lysis and immunogenic cell death of tumor cells, is a first step in a method as described herein. In some embodiments, a step of triggering cell lysis and immunogenic cell death of tumor cells is a first step in a method as described herein, and followed by a step of administering a combination formulation comprising immunotherapeutic agents as described herein.

The present disclosure recognizes that cryotherapy (such as cryoablation and cryolysis) can directly induce necrosis by damaging cell membranes and organelles via the formation of ice crystals, and indirectly through osmotic stress and ischemia from thrombosis of the microvasculature (Fraser and Gill 1967, Whittaker 1984, Karlsson, Cravalho et al. 1993, Hoffmann and Bischof 2002, Sabel 2009). Its mechanism of action has been attributed to mechanical forces and osmotic changes induced by water crystallization, and by ischemic effects of microvascular injury that contribute to coagulative necrosis of a tumor (Gage and Baust 1998). Upon thawing, necrotic tumor cells within an ice ball (e.g., an ice ball formed during cryolysis) release intact tumor antigens and DAMPs, such as cytokines, intracellular ATP, nuclear proteins and endogenous TLR ligands such as HMGB1 proteins (Sidana 2014). DAMPS stimulate an innate immune response and attract granulocytes, macrophages, and NK cells. DCs take up tumor antigens, migrate to a TDLN and initiate an adaptive immune response (mechanistically similar to an in-situ immunization) (den Brok, Sutmuller et al. 2006). Antigen loaded immature DCs are then activated and migrate to the TDLN where they process and display tumor antigens in context of MHC molecules and present antigens to T cells priming their activation and promoting the expansion of Th1 cytotoxic CD8+ T cells. Additionally, soluble antigens reach TDLNs where they can be loaded and cross-presented by DCs. Thus, controlled tumor lysis by cryolysis of a solid tumor is mechanistically similar to an in-situ immunization where many unique TAA/TSA from a heterogeneous population of tumor cells are released in conjunction with danger signals, which are able to prime and initiate an immune response to such antigens (Bastianpillai, Petrides et al. 2015, Yakkala, Chiang et al. 2019). Several animal experiments have confirmed an induction of systemic antitumor immune response by tumor cryolysis (Blackwood and Cooper 1972, Neel and DeSanto 1973, Bagley, Faraci et al. 1974, Faraci, Bagley et al. 1975, Faraci, Bagley et al. 1975, Muller, Micksche et al. 1985, Joosten, Muijen et al. 2001, Urano, Tanaka et al. 2003, Sabel and Nehs 2005, Sabel, Nehs et al. 2005, den Brok, Sutmuller et al. 2006, den Brok, Sutmuller et al. 2006, Sabel, Arora et al. 2006) (for review see (Sabel 2009)). These studies showed that cryotherapy (e.g., cryolysis) alone was able to increase the amount of tumor antigen loaded and mature DCs in TDLNs (den Brok, Sutmuller et al. 2006), higher levels of Th1 cytokines (IL2 and IFNγ), higher tumor rejection rates and increased protection from tumor rechallenge.

It is understood in the field that a threshold appears to exist with regard to antigenic stimulant required to support antitumor immunity, which requires elimination of most of a tumor mass (i.e., cryoablation) (Blackwood and Cooper 1972). Indeed, prostate cancer patients whose primary tumors were treated with cryoablation showed a decrease in metastatic burden, with an enhancement of this immune response after repeated cryotherapy treatments leading to cryogenic destruction of prostatic tissue (Soanes, Ablin et al. 1970, Soanes, Gonder et al. 1970, Ablin 1972). Also, patients with metastatic prostate cancer demonstrated palliation in bone pain after cryosurgery, with associated increase in IgM antibodies (Gursel, Roberts et al. 1973). Further, cryoablation of high-risk prostate cancer was shown to increase serum levels of TNFα and IFNγ four weeks post-tumor ablation with an increase in tumor specific CTLs and an increased Th1:Th2 cytokines ratio (Soanes, Ablin et al. 1970).

Notably, cryotherapy does not always lead to immune stimulation and sometimes it even results in immunosuppression and tumor growth promotion (Hanawa 1993, Shibata, Yamashita et al. 1998). While many studies demonstrate some degree of antitumor immune response post-cryosurgery, many studies failed to show any response. There are multiple possible reasons for these discrepancies. First, without wishing to be bound by theory, the rate of freezing is thought to play a role in determining immune-stimulating versus immunosuppressive effects of cryotherapies. A high freezing rate leads to formation of ice crystals, which results in necrotic immunogenic cell death, and is associated with increased expression of IFNγ and lower number of Tregs (Sabel, Su et al. 2010). On the contrary, with a slower freezing rate, which is observed in the periphery of an ice ball, cells die by osmotic shock, followed by apoptosis, which is less immunogenic and is correlated with an immunosuppressive increase in Treg cells, low IFNγ levels and a Th2 type of T cell response (e.g., TGF-ß and IL-10) (Sabel 2009, Sabel, Su et al. 2010). Thus, the ratio of necrosis to apoptosis are understood to play a critical role in determining the stimulating or suppressive nature of the immune response to cryotherapy (Sabel 2009, Ferguson, Choi et al. 2011). In addition, when a large amount of tumor is frozen, large quantities of immune complexes generated may cause ‘high zone tolerance’, a phenomenon where antigen overloading may lead to immunosuppression (Sabel 2009).

Partial cryolysis of tumor tissue, as performed in many embodiments of the present disclosure, fundamentally changes a tumor microenvironment within a treatment zone, achieving, among other things, a preponderance of tumor cells lysed with cell membranes ruptured (necrotic cell death), thereby releasing their cellular contents (including antigens, and damage associated molecular patterns (DAMPS), and other cellular debris) into the extracellular space, while also minimizing damage to lymphatic drainage of the tissue and denaturing of the released tumor proteins. Partial cryolysis as performed in many embodiments of the present disclosure results in a different outcome than that of a total tumor cryoablation. For example, unlike the more commonly practiced tumor cryoablation, which requires complete cell death of both an entire tumor and a surrounding margin of healthy tissue, partial cryolysis methods of the present disclosure result in only a preponderance of necrotic tumor cell death in a partial tumor treatment zone being lysed, with no healthy cell margin at all in a targeted treatment zone. A cryolysis method such as this would result in a failed cryoablation for several reasons, including only a portion of a tumor being lysed, no healthy surrounding tissue margin, and an extensive number of tumor cells remaining viable in a partial cryolysis treatment zone.

The present disclosure appreciates that partial tumor lysis that results in necrotic cell death via ruptured cell membranes can potentially be achieved via many modalities including radiation, radiofrequency, high intensity focused ultrasound (HIFU), microwave, oncolytic viruses, oncolytic chemical agents, oncolytic peptides, and cryolysis. In many embodiments, cryolysis is favored over other modalities because of its efficiency, repeatability, superior elicitation of immune response than heat-based modalities, quality of released proteins (minimal denaturing), overall safety profile and lack of off-target effects. In some embodiments of the present disclosure, cryolysis is mediated by an intratumoral cryolysis device, followed by at least one cycle of freezing and thawing using standard and approved cryolysis devices and probes. In some preferred embodiments, cryolysis is administered as a first step in methods described herein.

In some embodiments of the present disclosure, to mediate cryolysis a cryoprobe is inserted into a tumor (e.g., intratumorally). Once in position in a tumor a cryoprobe is cooled to a temperature that creates an ice ball within said tumor that has a diameter of about 10 mm to about 14 mm. In some embodiments, an ice ball within a tumor has a diameter of about 8 mm to about 14 mm. In some embodiments, an ice ball within a tumor has a diameter of about 8 mm to about 12 mm. In some embodiments, an ice ball within a tumor has a diameter of about 8 mm to about mm. In some embodiments, an ice ball within a tumor has a diameter of about 12 mm to about 16 mm. In some embodiments, an ice ball within a tumor has a diameter of about 13 to about 15 mm. In some embodiments, an ice ball within a tumor has a diameter of about 14 mm to about 18 mm. In some embodiments, an ice ball within a tumor has a diameter of about 14 mm to about 30 mm. In some embodiments, an ice ball within a tumor has a diameter of about 16 mm to about 30 mm. In some embodiments, an ice ball within a tumor has a diameter of about 18 mm to about 30 mm. In some embodiments, an ice ball within a tumor has a diameter of about 20 mm to about 30 mm. In some embodiments, an ice ball within a tumor has a diameter of about 25 mm to about 30 mm. In some embodiments, an ice ball within a tumor has a diameter of about 14 mm to about 25 mm. In some embodiments, an ice ball within a tumor has a diameter of about 14 mm to about 20 mm. In some embodiments, an ice ball within a tumor has a diameter of about 14 mm to about 15 mm. In some embodiments, an ice ball within a tumor has a diameter of about 13 mm to about 14 mm. In some embodiments, an ice ball within a tumor has a diameter of about 8 mm. In some embodiments, an ice ball within a tumor has a diameter of about 9 mm. In some embodiments, an ice ball within a tumor has a diameter of about 10 mm. In some embodiments, an ice ball within a tumor has a diameter of about 11 mm. In some embodiments, an ice ball within a tumor has a diameter of about 12 mm. In some embodiments, an ice ball within a tumor has a diameter of about 13 mm. In some embodiments, an ice ball within a tumor has a diameter of about 14 mm. In some embodiments, an ice ball within a tumor has a diameter of about 15 mm. In some embodiments, an ice ball within a tumor has a diameter of about 16 mm. In some embodiments, an ice ball within a tumor has a diameter of about 17 mm. In some embodiments, an ice ball within a tumor has a diameter of about 18 mm. In some embodiments, an ice ball within a tumor has a diameter of about 19 mm. In some embodiments, an ice ball within a tumor has a diameter of about 20 mm. In some embodiments, an ice ball within a tumor has a diameter of about 21 mm. In some embodiments, an ice ball within a tumor has a diameter of about 22 mm. In some embodiments, an ice ball within a tumor has a diameter of about 23 mm. In some embodiments, an ice ball within a tumor has a diameter of about 24 mm. In some embodiments, an ice ball within a tumor has a diameter of about mm. In some embodiments, an ice ball within a tumor has a diameter of about 30 mm.

In some embodiments, an ice ball, as formed in methods described herein, has a temperature gradient within the ice ball where the temperature of ice near a cryoprobe surface is about the same as the cryoprobe surface. Further, as distance from a cryoprobe is increased, temperature within the ice ball changes and approaches 0° C. near an ice ball/tissue interface (also referred to as a leading edge). Due to this temperature change, in some embodiments, temperatures generated at a cryoprobe are below −40° C. to −60° C. In some embodiments, temperatures generated at a cryoprobe are below −40° C. In some embodiments, temperatures generated at a cryoprobe are below −60° C. In some embodiments, temperatures generated at a cryoprobe are below −80° C. In some embodiments, temperatures generated at a cryoprobe are below −100° C. In some embodiments, temperatures generated at a cryoprobe are below −120° C. In some embodiments, temperatures generated at a cryoprobe are below −140° C. In some embodiments, cooling of a cryoprobe creates an ice ball where the majority of the ice has a temperature that is approximately −40° C. In some embodiments, lethal ice (e.g., ice that causes cell death by necrosis, apoptosis, or osmotic shock) in an ice ball (at about −40° C. or colder) resides about 5 mm from a leading edge (ice at the ice ball/tissue interface that is at temperature of about 0° C.). In some embodiments, lethal ice in an ice ball resides about 1 mm, about 2 mm, about 3 mm, about 4 mm, or about 5 mm from a leading edge. After freezing, a frozen tumor tissue ice ball formed by a cryoprobe is allowed to thaw passively (no heat cycle for the cryoprobe) and completely, which is automatically determined by timing and, which may further be verified by any suitable imaging method known in the field (e.g., thermocouples, ultrasound).

In some embodiments of the present disclosure, a partial tumor cryolysis treatment zone size is the same regardless of the tumor size being treated and is defined by the size of an ice ball that is generated. In some preferred embodiments, the size of an ice ball diameter ranges from about 8 mm to about 30 mm. In some embodiments, the size of an ice ball diameter ranges from about 10 mm to about 30 mm. In some embodiments, the size of an ice ball diameter ranges from about 12 mm to about 30 mm. In some embodiments, the size of an ice ball diameter ranges from about 14 mm to about 30 mm. In some embodiments, the size of an ice ball diameter ranges from about 16 mm to about 30 mm. In some embodiments, the size of an ice ball diameter ranges from about 18 mm to about 30 mm. In some embodiments, the size of an ice ball diameter ranges from about 20 mm to about 30 mm. In some embodiments, the size of an ice ball diameter ranges from about 25 mm to about 30 mm. In some embodiments, the size of an ice ball diameter ranges from about 8 mm to about 25 mm. In some embodiments, the size of an ice ball diameter ranges from about 8 mm to about 20 mm. In some embodiments, the size of an ice ball diameter ranges from about 8 mm to about 18 mm. In some embodiments, the size of an ice ball diameter ranges from about 8 mm to about 16 mm. In some embodiments, the size of an ice ball diameter ranges from about 8 mm to about 14 mm. In some embodiments, the size of an ice ball diameter ranges from about 8 mm to about 12 mm. In some preferred embodiments, size of an ice ball diameter ranges from about 12 mm to about 16 mm. In some embodiments, the size of an ice ball diameter ranges from about 10 mm to about 14 mm. In some embodiments, the size of an ice ball diameter ranges from about 11 mm to about 15 mm. In some embodiments, the size of an ice ball diameter ranges from about 13 mm to about 15 mm.

In some preferred embodiments, an ice ball has a diameter of about 14 mm, which results in a spherical volume of partially lysed tissue of approximately 1.44 ml. In some embodiments, an ice ball has a diameter which results in a spherical volume of partially lysed tissue of approximately 1.2 ml to approximately 1.6 ml. In some embodiments, an ice ball has a diameter which results in a spherical volume of partially lysed tissue of approximately 0.3 ml to approximately 14 ml. In some embodiments, an ice ball has a diameter which results in a spherical volume of partially lysed tissue of approximately 0.3 ml to approximately 10 ml. In some embodiments, an ice ball has a diameter which results in a spherical volume of partially lysed tissue of approximately 0.3 ml to approximately 8 ml. In some embodiments, an ice ball has a diameter which results in a spherical volume of partially lysed tissue of approximately 0.3 ml to approximately 4 ml. In some embodiments, an ice ball has a diameter which results in a spherical volume of partially lysed tissue of approximately 0.3 ml to approximately 2 ml. In some embodiments, an ice ball has a diameter which results in a spherical volume of partially lysed tissue of approximately 0.3 ml to approximately 1.4 ml. In some embodiments, an ice ball has a diameter which results in a spherical volume of partially lysed tissue of approximately 0.3 ml to approximately 1 ml. In some embodiments, an ice ball has a diameter which results in a spherical volume of partially lysed tissue of approximately 1 ml to approximately 14 ml. In some embodiments, an ice ball has a diameter which results in a spherical volume of partially lysed tissue of approximately 1.4 ml to approximately 14 ml. In some embodiments, an ice ball has a diameter which results in a spherical volume of partially lysed tissue of approximately 2 ml to approximately 14 ml. In some embodiments, an ice ball has a diameter which results in a spherical volume of partially lysed tissue of approximately 4 ml to approximately 14 ml. In some embodiments, an ice ball has a diameter which results in a spherical volume of partially lysed tissue of approximately 8 ml to approximately 14 ml. In some embodiments, an ice ball has a diameter which results in a spherical volume of partially lysed tissue of approximately 10 ml to approximately 14 ml.

In some embodiments, during a cycle of cryolysis, an ice ball is generated by cooling the temperature at a cryoprobe to about −40° C. to about −60° C., or colder in about 1 minute or less, and continuing to cool the temperature to about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder for a total time of about 5 minutes, about 4.5 minutes, about 4 minutes, about 3.5 minutes, about 3 minutes, about 2.5 minutes, about 2 minutes, about 1.75 minutes, about 1.5 minutes, about 1.25 minutes, about 1 minute, about 50 seconds, about 40 seconds, about 30 seconds, about 20 seconds, or about 10 seconds. In some embodiments, an ice ball is generated by cooling the temperature at a cryoprobe to about −40° C. to about −60° C., or colder in about 1 minute or less, and continuing to cool the temperature to about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder for a total time of about 5 minutes. In some embodiments, an ice ball is generated by cooling the temperature at a cryoprobe to about −40° C. to about −60° C., or colder in about 1 minute or less, and continuing to cool the temperature to about −120° C. to about −150° C. or colder for a total time of about 5 minutes. In some embodiments, an ice ball is generated by cooling the temperature at a cryoprobe to about −40° C. to about −60° C., or colder in about 1 minute or less, and continuing to cool the temperature to about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder for a total time of about 2 minutes. In some embodiments, an ice ball is generated by cooling the temperature at a cryoprobe to about −40° C. to about −60° C., or colder in about 1 minute or less, and continuing to cool the temperature to about −120° C. to about −150° C. or colder for a total time of about 2 minutes.

In some embodiments, temperatures generated at a cryoprobe reach about −40° C. to about −60° C. or colder in about 1 minute. In some embodiments, temperatures generated at a cryoprobe reach about −40° C. to about −60° C. or colder in about 50 seconds. In some embodiments, temperatures generated at a cryoprobe reach about −40° C. to about −60° C. or colder in about 40 seconds. In some embodiments, temperatures generated at a cryoprobe reach about −40° C. to about −60° C. or colder in about 30 seconds. In some embodiments, temperatures generated at a cryoprobe reach about −40° C. to about −60° C. or colder in about 20 seconds. In some embodiments, temperatures generated at a cryoprobe reach about −40° C. to about −60° C. or colder in about 10 seconds. In some embodiments, temperatures generated at a cryoprobe reach about −40° C. to about −60° C. or colder in about 1 minute or less. In some embodiments, temperatures generated at a cryoprobe reach about −40° C. to about −60° C. or colder in about 50 seconds or less. In some embodiments, temperatures generated at a cryoprobe reach about −40° C. to about −60° C. or colder in about 40 seconds or less. In some embodiments, temperatures generated at a cryoprobe reach about −40° C. to about −60° C. or colder in about 30 seconds or less. In some embodiments, temperatures generated at a cryoprobe reach about −40° C. to about −60° C. or colder in about 20 seconds or less. In some embodiments, temperatures generated at a cryoprobe reach about −40° C. to about −60° C. or colder in about 10 seconds or less.

In some embodiments, temperatures generated at a cryoprobe reach about −60° C. to about −70° C., about −40° C. to about −70° C., about −50° C. to about −70° C., about −60° C. to about −80° C., about −70° C. to about −90° C., about −80° C. to about −100° C., about −90° C. to about −110° C., about −100° C. to about −120° C., about −110° C. to about −130° C., about −120° C. to about −150° C., or colder in about 5 minutes, about 4.5 minutes, about 4 minutes, about 3.5 minutes, about 3 minutes, about 2.5 minutes, about 2 minutes, about 1.75 minutes, about 1.5 minutes, about 1.25 minutes, about 1 minute, about 50 seconds, about 40 seconds, about 30 seconds, about 20 seconds, or about 10 seconds. In some embodiments, temperatures generated at a cryoprobe reach about −60° C. to about −70° C., about −40° C. to about −70° C., about −50° C. to about −70° C., about −60° C. to about −80° C., about −70° C. to about −90° C., about −80° C. to about −100° C., about −90° C. to about −110° C., about −100° C. to about −120° C., about −110° C. to about −130° C., about −120° C. to about −150° C., or colder in about 5 minutes or less, about 4.5 minutes or less, about 4 minutes or less, about 3.5 minutes or less, about 3 minutes or less, about 2.5 minutes or less, about 2 minutes or less, about 1.75 minutes or less, about 1.5 minutes or less, about 1.25 minutes or less, about 1 minute or less, about 50 seconds or less, about 40 seconds or less, about 30 seconds or less, about 20 seconds or less, or about 10 seconds or less.

In some embodiments, an ice ball generation cycle time is predetermined and is automatically ended a few seconds after an ice ball has reached its predetermined maximum size of about 8 mm to about 30 mm, about 10 mm to about 30 mm, about 12 mm to about 30 mm, about 14 mm to about 30 mm, about 16 mm to about 30 mm, about 18 mm to about 30 mm, about 20 mm to about 30 mm, about 25 mm to about 30 mm, about 8 mm to about 25 mm, about 8 mm to about 20 mm, about 8 mm to about 18 mm, about 8 mm to about 16 mm, about 8 mm to about 14 mm, about 8 mm to about 12 mm, about 8 mm to about 10 mm, about 12 mm to about 16 mm, about 13 mm to about 15 mm, or about 14 mm in diameter. In some embodiments, an ice ball generation cycle time is predetermined and is automatically ended a few seconds after an ice ball has reached its predetermined maximum size of about 8 mm to about 30 mm, about 12 mm to about 16 mm, or about 14 mm in diameter.

In some embodiments, cooling of a cryoprobe creates an ice ball where the majority of the ice has a temperature that is approximately −40° C. or colder, approximately −45° C. or colder, approximately −50° C. or colder, approximately −55° C. or colder, approximately −60° C. or colder, approximately −65° C., approximately −70° C., approximately −80° C. or colder, approximately −90° C. or colder, approximately −100° C. or colder, approximately −110° C. or colder, approximately −120° C. or colder, approximately −130° C. or colder, approximately −140° C. or colder, or approximately −150° C., or colder. In some embodiments, cooling of a cryoprobe creates an ice ball where the majority of the ice has a temperature that is approximately −40° C. or colder. In some embodiments, cooling of a cryoprobe creates an ice ball where the majority of the ice has a temperature that is approximately −60° C. or colder.

In some embodiments, an ice ball comprises an approximately 8 mm, approximately 9 mm, approximately 10 mm, approximately 11 mm, approximately 12 mm, approximately 13 mm, approximately 14 mm, or approximately 15 mm diameter region within that has a temperature of about −40° C. or colder, about −50° C. or colder, about −60° C. or colder, about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder. In some embodiments, an ice ball comprises an approximately 10 mm diameter within that has a temperature of about −40° C., about −50° C., about −60° C. or colder, about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder. In some embodiments, an ice ball comprises an approximately 10 mm diameter within that has a temperature of about −40° C. or colder.

In some embodiments, after an ice ball has reached its predetermined maximum size, a step of thawing (e.g., passive thawing) is performed. In some embodiments, a step of passive thawing lasts for about 10 minutes, about 9.5 minutes, about 9 minutes, about 8.5 minutes, about 8 minutes, about 7.5 minutes, about 7 minutes, about 6.5 minutes, about 6 minutes, about 5.5 minutes, about 5 minutes, about 4.5 minutes, about 4 minutes, about 3.5 minutes, about 3 minutes, about 2.75 minutes, about 2.5 minutes, about 2.25 minutes, about 2 minutes, about 1.75 minutes, about 1.5 minutes, about 1.25 minutes, or about 1 minute. In some embodiments, a step of passive thawing lasts for less than about 10 minutes, less than about 9.5 minutes, less than about 9 minutes, less than about 8.5 minutes, less than about 8 minutes, less than about 7.5 minutes, less than about 7 minutes, less than about 6.5 minutes, less than about 6 minutes, less than about 5.5 minutes, less than about 5 minutes, less than about 4.5 minutes, less than about 4 minutes, less than about 3.5 minutes, less than about 3 minutes, less than about 2.75 minutes, less than about 2.5 minutes, less than about 2.25 minutes, less than about 2 minutes, less than about 1.75 minutes, less than about 1.5 minutes, less than about 1.25 minutes, or less than about 1 minute. In some embodiments, a step of passive thawing lasts for about 5 minutes. In some embodiments, a step of passive thawing lasts for about 2 minutes. In some embodiments, a step of passive thawing lasts until an ice ball has dissipated (e.g., as judged by a suitable visualization method).

In some embodiments of the disclosure, a second cycle of cryolysis repeated after a passive thawing of frozen tissue has taken place and is verified by any suitable imaging method known in the field. In some embodiments, a third cycle of cryolysis is repeated after a passive thawing of frozen tissue has taken place and is verified by any suitable imaging method known in the field.

Cryolytic cycles described herein are intended to produce a tissue effect in a tumor treatment zone whereby a preponderance of tumor cells are lysed, (i.e., their cell membranes are ruptured) thereby releasing their cellular contents into the extracellular space. Freeze/thaw cycles described herein are intended to not damage lymphatic drainage in a tumor tissue in and around a treatment zone. Frozen tumor tissue in an ice ball (e.g., an ice ball formed by a cryoprobe) is allowed to thaw passively (no heat cycle for a cryoprobe) and completely, which is automatically determined by timing and, which may be verified by any suitable imaging method known in the field.

In some embodiments, a cryolysis therapy administered according to the present disclosure causes necrotic cell death in a zone of tumor tissue with the zone being approximately 8 mm, approximately 9 mm, approximately 10 mm, approximately 11 mm, approximately 12 mm, approximately 13 mm, approximately 14 mm, or approximately 15 mm in diameter. In some embodiments, a cryolysis therapy causes necrotic cell death in a zone of tumor tissue with the zone being approximately 12 mm to approximately 16 mm in diameter. In some embodiments, a cryolysis therapy causes necrotic cell death in a zone of tumor tissue with the zone being approximately 13 mm to approximately 15 mm in diameter. In some embodiments, a cryolysis therapy causes necrotic cell death in a zone of tumor tissue with the zone being approximately 14 mm in diameter.

In some embodiments of the present disclosure, a selected tumor site is prepped for a precision image guided percutaneous approach with a coaxial cryoprobe/infusion needle system (SCINS). A SCINS is inserted via standard precision image guided percutaneous technique with the tip of the SCINS being located precisely so that a resulting cryolysis treatment zone volume will reside completely within a selected tumor. The length of a cryolysis zone with a SCINS can be visualized under imaging as length of a cryoprobe that is exposed from the tip of a coaxial infusion needle.

In some embodiments, a cryolysis device is set to a duty cycle that allows for the generation of a cryoprobe temperature of about −40° C., about −50° C., about −60° C., or colder in about 5 minutes, about 4.5 minutes, about 4 minutes, about 3.5 minutes, about 3 minutes, about 2.75 minutes, about 2.5 minutes, about 2.25 minutes, about 2 minutes, about 1.75 minutes, about 1.5 minutes, about 1.25 minutes, about 1 minute, about 50 seconds, about 40 seconds, about 30 seconds, about 20 seconds, or about 10 seconds. In some embodiments, a cryolysis device is set to a duty cycle that allows for the generation of a cryoprobe temperature of about −40° C., about −50° C., about −60° C., or colder in about 5 minutes or less, about 4.5 minutes or less, about 4 minutes or less, about 3.5 minutes or less, about 3 minutes or less, about 2.75 minutes, about 2.5 minutes or less, about 2.25 minutes or less, about 2 minutes or less, about 1.75 minutes or less, about 1.5 minutes or less, about 1.25 minutes or less, or about 1 minute or less. In some embodiments, a cryolysis device is set to a duty cycle that allows for the generation of a cryoprobe temperature of about −40° C. or colder in less than about 1 minute. In some embodiments, a cryolysis device is set to a duty cycle that allows for the generation of a cryoprobe temperature of about −60° C. or colder in less than about 1 minute. In some embodiments, a cryolysis device is set to a duty cycle of about 100%, about 99%, about 98%, about 97%, about 96%, about 95%, about 94%, about 93%, about 92%, about 91%, about 90%, about 80%, or about 70%. In some embodiments, a cryolysis device is set to a duty cycle of at least about 100%, at least about 99%, at least about 98%, at least about 97%, at least about 96%, at least about 95%, at least about 94%, at least about 93%, at least about 92%, at least about 91%, at least about 90%, at least about 80%, or at least about 70%. In some embodiments, a cryolysis device is set to a duty cycle of about 100%.

Administration of Combination Formulations

Methods and compositions of the present disclosure are based on an intratumoral administration of a TLR9 agonist, an anti-CD40 agonistic monoclonal antibody, an anti-CTLA4 monoclonal antibody and either an agonistic anti-OX40 monoclonal antibody or an anti-PD1 monoclonal antibody. These combinations of immune-stimulating reagents are designed to promote and favor an immunogenic antitumor response following tumor cell lysis mediated by cryolysis, and to ensure that the outcome of cryolysis is pro-inflammatory, avoiding those instances when it can potentially be immunosuppressive.

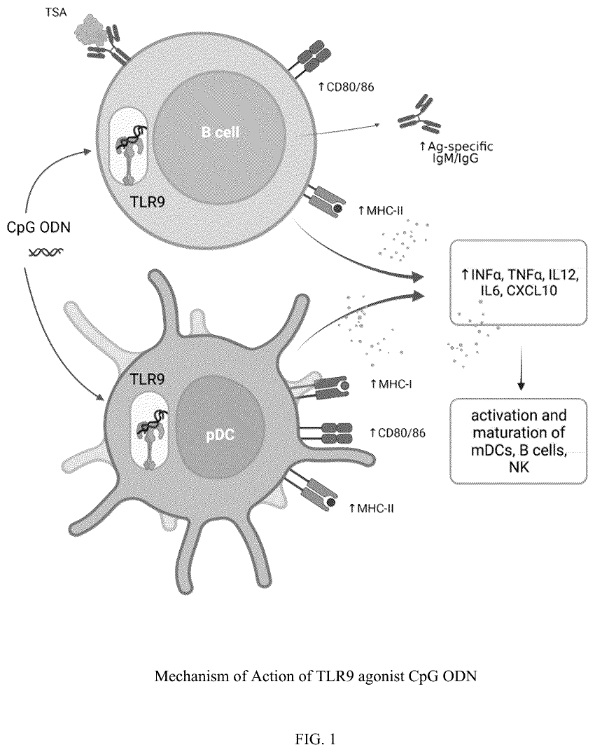

TLR9 is an intracellular pattern recognition receptor present in the endosomal compartment of plasmacytoid dendritic cells, macrophages, natural killer cells, and other APCs in mice (Karapetyan, Luke et al. 2020). In humans, TLR9 is primarily expressed in B cells and pDCs (Krieg 2007). The physiologic ligands for TLR9 are bacterial dsDNA fragments with unmethylated cytidine phosphate guanosine (CpG) dinucleotides. TLR9 agonists are artificial oligonucleotides bearing unmethylated CpG motifs. They can induce a signaling cascade that leads to transcriptional programs that result in inflammatory processes, enhanced killing of cancer cells as well as in the generation of adaptive immune responses. Compelling data suggest that intratumoral administration of TLR9 agonists improves APC activation, in particular pDCs in TDLNs resulting in proinflammatory Th1 cytokine release such as TNFα, IFNγ, IL2, IL-6, IL-12 and type I IFN genes (Suek, Campesato et al. 2019, Karapetyan, Luke et al. 2020). IFNα has direct effects on tumors, including inhibition of angiogenesis, antiproliferative effects and increase in MHC-I expression by tumor cells, which enhances target visibility and facilitates killing of tumor cells by T cells (Suek, Campesato et al. 2019). In addition, IFNα enhances NK killing ability and maturation of conventional DCs (Hervas-Stubbs, Perez-Gracia et al. 2011). Additionally, TLR9 agonists can increase expression of co-stimulatory molecules CD80 and CD86, increase in IP10 (CXCL10) a chemoattractant of monocytes, macrophages, DCs, T cells, NK cells, and increase expression of the lymph node homing signal CCR7, which ultimately results in increased T cell priming and tumor rejection, and in some instances, potent abscopal effects in distant non-treated tumors (Suek, Campesato et al. 2019, Karapetyan, Luke et al. 2020) (FIG. 1).

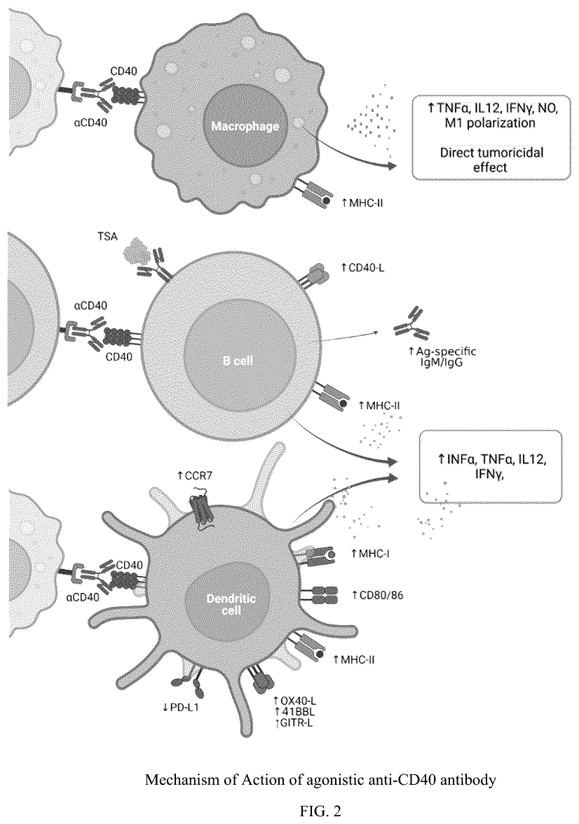

CD40 is a membrane molecule a member of the TNFR family, expressed in APCs such as DCs, B cells, monocytes and macrophages. The key physiological function of the CD40/CD40L pathway is mediated by CD40 ligation on APCs, especially dendritic cells (DCs) by CD40L expressed primarily by CD4 helper T cells (Schonbeck and Libby 2001, Vonderheide and Glennie 2013, Vonderheide 2020). CD40 is not a direct T-cell agonist, however, its capacity to increase antigen presentation enhances T-cell activation indirectly. CD40 is expressed in APCs such as DCs, B cells and macrophages, and its ligation by CD40L expressed by helper T cells or an agonistic mAb enables activation of the APCs by improving their antigen presentation ability via the upregulation of HLA class II and co-stimulatory molecules CD80/86, and by increasing the T cell priming ability of APCs via upregulation of OX40-L, 4-1BB-L, GITR-L, while downregulating immunosuppressive molecules such as PD-L1. CD40 agonism also increases the release of various Th1 cytokines by DCs (IL-1β, IL-6, IL-8, IL-12, TNFα and IFNg), which enhance the cytotoxic response (Schonbeck and Libby 2001, Vonderheide and Glennie 2013, Vonderheide 2020). In particular, αCD40 agonistic mAbs elicit IL-12 secretion by APCs, which i) downregulates expression of CTLA4 and PD1 on Tregs, making them less immunosuppressive, and ii) downregulates PD1 on effector CD8+ T cells, which restores response of T cells to αPD1 mAbs (Ngiow, Young et al. 2016). CD40 also affects the expression of proapoptotic and antiapoptotic genes (Bcl-XL) on different types of cells (Choi, Shi et al. 2020). In B cells, αCD40 agonistic mAb can upregulate HLA-II, CD40L, Th1 cytokine production and elicit secretion of antigen-specific IgM/IgG. Agonistic αCD40 mAbs can also induce secretion of cytokines that are crucial to macrophage cross-priming and cytotoxic function, activating macrophages to directly kill tumor cells in a T cell independent manner. It has been shown that αCD40 antibodies of the IgG2 isotype have potent CD40 agonistic activity and can mimic the signals from CD40L-expressing helper CD4+ T cells to activate APCs, licensing them to prime CD8+ T cells (Vonderheide and Glennie 2013, Vonderheide 2020, Yu, Chan et al. 2020). CD40 activation requires oligomerization of the CD40 receptor to induce agonistic effects. Oligomerization of the receptor can be enhanced in trans by interaction of the Fc domain of the αCD40 agonistic mAb with Fc receptor of neighboring cells (White, Chan et al. 2011) (FIG. 2).

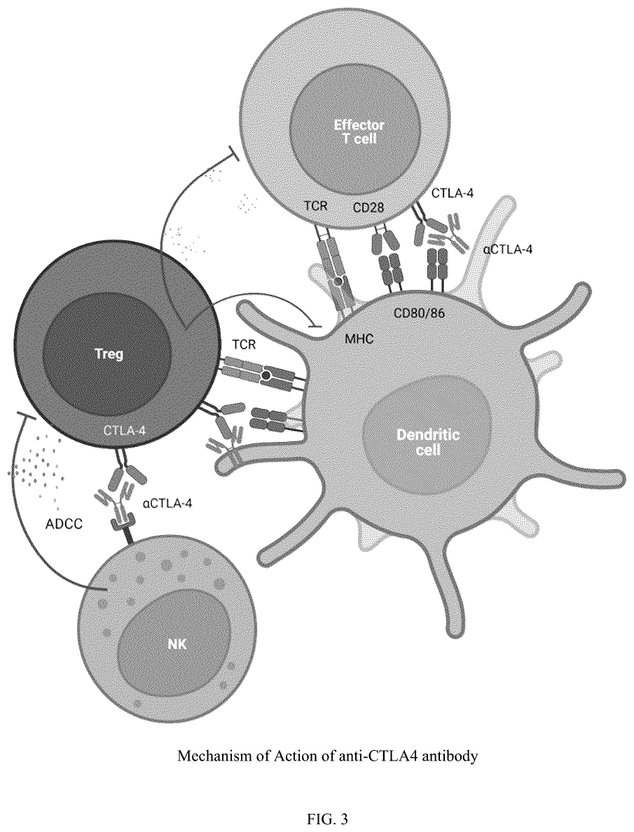

CTLA4 is a suppressive molecule expressed constitutively in Tregs and inducible in effector CD4+ and CD8+ T cells following their TCR engagement and activation (Wei, Duffy et al. 2018). The level of expression of CTLA4 in the immune synapse correlates to the strength of TCR affinity for the MHCI/peptides complexes (Buchbinder and Desai 2016). CTLA4 binds to CD80 and CD86 co-stimulatory molecules on the APCs with higher affinity than the co-stimulatory ligand CD28 expressed on T cells. Thus, CTLA4 negatively outcompetes the positive activation signal mediated by CD28 after binding to CD80/86. The competition between CD28 and CTLA4 has the function of controlling peripheral tolerance to highly reactive T cell clones by allowing the expansion of those only those clones with moderate TCR/MHC-I/peptide affinity and reducing the proliferation of those clones with very strong TCR/MHC-I/peptide affinity (Buchbinder and Desai 2016). In addition to this cell intrinsic mechanism of suppression, where CTLA4 and CD28 from the same T cell compete with each other for binding to CD80/86, CTLA4 has a cell extrinsic suppressive mechanism, where CTLA4 expressed by Tregs blocks CD80/86 on the surface of the APC, as well as sequesters CD80/86 by trans-endocytosis (Qureshi, Zheng et al. 2011), thereby reducing its availability and preventing their interaction with CD28 on the T cell being activated by such APC (Buchbinder and Desai 2016, Wei, Duffy et al. 2018). Based on the above mechanism, CTLA4 blockade with αCTLA4 mAbs is expected to increase immune activation by interfering with both of these cell-intrinsic and cell-extrinsic mechanisms. First, blocking CTLA4 on activated T cells and on Tregs is supposed to allow the activation of effector T cells by increasing the co-stimulatory interaction between CD80/86 and CD28, regardless of their TCR affinity and specificity. This leads to the activation and proliferation of a higher number of effector T cell clones, which would contribute to antitumor activity by enhancing the priming of T cells and by broadening the TCR repertoire and spectrum of tumor antigens that CD8+ T cells react to (Cha, Klinger et al. 2014, Kvistborg, Philips et al. 2014, Robert, Harview et al. 2014, Robert, Tsoi et al. 2014). Second, CTLA4 blockade leads to the expansion of tumor neoantigen-specific CD8+ T cells within the tumor microenvironment (Fehlings, Simoni et al. 2017) and to the expansion of a particular subset of exhausted Th1 CD4+ T cells (Wei, Levine et al. 2017). Third, there is evidence showing that αCTLA4 mAb may contribute to antitumor immunity via a second mechanism involving ADCC-mediated depletion of Tregs, which can not only enhance T cell priming in the TDLN but also reset the cold TME by deletion of tumor infiltrating Tregs (Wei, Duffy et al. 2018). Therefore, αCTLA4 mAbs mediate their function by a dual mechanism involving Treg depletion by Fc-dependent ADCC and by blocking the inhibition of costimulatory signals in the APC provided by CD80/86 on CD28+ T cells, thus favoring the CD28-mediated activation of T cells (Tang, Du et al. 2018, Wei, Duffy et al. 2018). Regarding the site of action at which CTLA4 blockade exerts its pharmacological effect, it is thought that the blockade of CTLA4 most likely impacts the early stage of T cell activation in the draining lymph nodes when CTLA4+ Tregs remove CD80/CD86 from the surface of APCs by trans-endocytosis and compete with CD28 for binding to CD80/86 (Qureshi, Zheng et al. 2011). In addition, CTLA4 blockade may also take effect at the tumor site as exhausted CTLA4+ T cells and Tregs can accumulate within the tumor microenvironment (Curran, Montalvo et al. 2010). Blocking CTLA4 with antagonistic mAbs has been shown to increase the ratio of CD8:Tregs in the tumor microenvironment (FIG. 3).

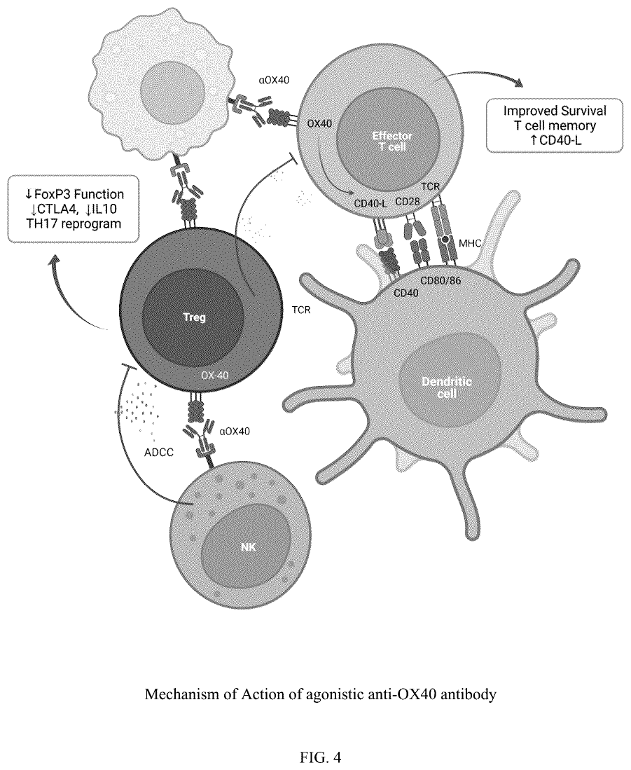

OX40 is a member of the TNFR superfamily and is expressed by effector CD4+ and CD8+ T cells, Tregs, NKT and NK within 1-4 days after TCR engagement. Ligation of OX40 by OX40L expressed by APCs or by agonistic anti-OX40 mAbs promotes different signals that enhance the survival of Teff cells and generation of memory T cells (Croft 2010). The immuno-pharmacologic effects include the promotion of cytokine production, proliferation, and upregulation of CD40L, which can help stimulate DCs via the CD40/CD40L axis (Colombo 2017). Therefore, agonistic anti-OX40 mAbs such as YH002 mimic the function of activated APCs on effector T cell activation. Clustering and trimerization of the OX40 receptor molecules mediated by many agonistic αOX40 mAb require the Fc function of the antibody and accessory cells that express the appropriate Fc receptor molecules, such as NKs, DCs, macrophages or B cells (Zhang, Armstrong et al. 2017, Kuang, Jing et al. 2020). In addition of having an activating function on Teff cells, agonistic αOX40 mAbs can inhibit the generation of Tregs and the suppressive activity of Tregs through direct inhibition of FoxP3 expression (Zhang, Xiao et al. 2018), which leads to reduced expression of the checkpoint inhibitory molecule CTLA4, thus allowing better co-stimulation of Teff cells via the CD28/CD80 axis. Furthermore, inhibition of FoxP3 on Tregs leads to reduction of IL10 secretion by Tregs, which favors better signaling between CD40 in DCs and CD40L on memory T cells (Burocchi, Pittoni et al. 2011). Moreover, agonistic αOX40 mAbs of the IgG1 isotype also can mediate depletion of Tregs by ADCC, particularly for intratumoral Tregs, which express high levels of OX40, and can favor reprogramming of Tregs into TH17 T cells (Piconese, Valzasina et al. 2008, Croft 2010, Aspeslagh, Postel-Vinay et al. 2016, Colombo 2017, Deng, Zhao et al. 2019, Alves Costa Silva, Facchinetti et al. 2020, Choi, Shi et al. 2020) (FIG. 4).

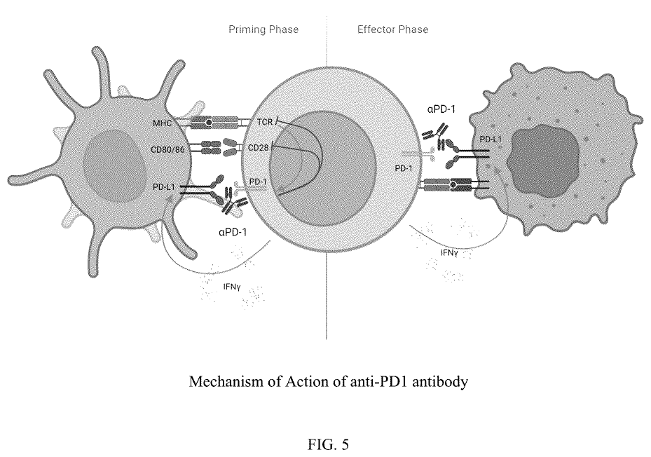

PD-1 is expressed on activated T cells following TCR-mediated antigen stimulation as well as on B cells, NK cells and monocytes. PD1 function is to control T cell activation in peripheral tissues to prevent tissue damage by ongoing inflammation. Prolonged or high levels of antigen exposure, such as in advanced cancer, leads to T cell exhaustion mediated by PD1. During T cell priming, PD-L1 is induced in APCs by IFNγ as a counterregulatory mechanism following T cell activation (Keir, Butte et al. 2008). Ligation of PD-1 with PD-L1 in the context of TCR activation leads to phosphorylation of PD1 by Lck tyrosine kinase, followed by recruitment of the SHP2 phosphatase to the intracellular domain of PD1, which in turn dephosphorylates and inactivates both the TCR and CD28, thus blocking the stimulatory and co-stimulatory downstream activation signaling that take place during T cell priming (Buchbinder and Desai 2016, Wei, Duffy et al. 2018). Similarly, during the effector phase in the tumor, PD-L1 is induced by IFNγ and expressed by tumor cells. Ligation of PD-L1 on PD1+ Teff cells that recognize tumor antigens presented by MHC-I via the TCR, recruits SHP2, which inactivates TCR signaling, reduces cytokine production and induces T cell apoptosis or exhaustion and anergy of PD1+TILs, resulting in exclusion of Teff cells from infiltrating the tumor (Keir, Butte et al. 2008, Shrimali, Janik et al. 2015). Blocking of PD1 with antagonistic mAbs works by restoring the immune function of exhausted T cells in the periphery (Shrimali, Janik et al. 2015) and can potentiate tumor killing by various immune mechanisms including direct inhibition of T-cell apoptosis leading to revived T-cell effector function, enhanced APC-mediated T-cell activation, downregulation of the effects of Treg-mediated suppression, downregulation of immune suppressive cytokines IL-10 and TGFβ and upregulation of IL-2 and IFN-γ, while providing resistance to the inducible expression of PD-L1 in the tumor (Wei, Duffy et al. 2018). Clinical evidence supports a model in which blockade of the PD1 signaling axis is most effective in tumors in which an endogenous T-cell response has already been elicited but is being suppressed through PD1 engagement by its ligands PD-L1 and PD-L2. However, the response to αPD1 mAb in PD-L1(−) tumors indicates that the presence of a preexisting immune response is not always an absolute requirement for tumor rejection after αPD1 therapy (FIG. 5).

Preclinical results have shown a broad spectrum of responses in different tumor models when combining αPD1 mAbs with agonistic αOX40 mAb (Guo, Wang et al. 2014, Vilgelm, Johnson et al. 2016, Messenheimer, Jensen et al. 2017, Shrimali, Ahmad et al. 2017). These responses range from synergistic to antagonistic effects, with the timing at which these two mAbs are dosed seemingly playing an important role in the type of response. For at least these reasons, the methods and compositions of the present disclosure do not combine the simultaneous administration of an agonistic anti-OX40 antibody with an antagonistic αPD1 mAb.

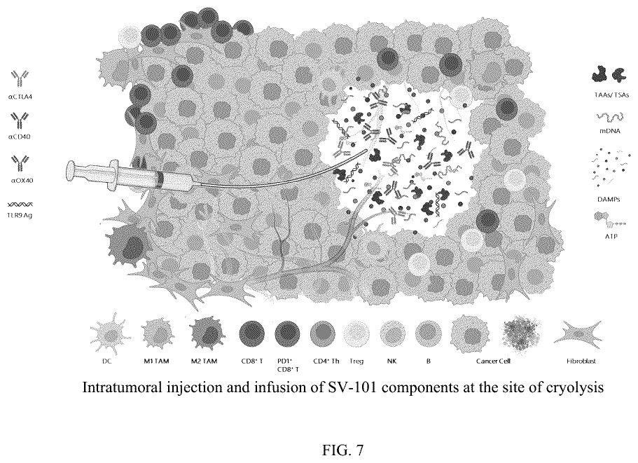

In some embodiments, methods of the present disclosure comprise a step of intratumoral administration of SV-101, a combination formulation of immune-activating therapeutic agents comprising a TLR9 agonist, an αCD40 agonist, an αOX40 agonist, and an αCTLA4 checkpoint blockade inhibitor. In some embodiments, methods of the present disclosure comprise a step of intratumoral administration of an SV-101 combination formulation consisting of a TLR9 agonist, an αCD40 agonist, an αOX40 agonist, and an αCTLA4 checkpoint blockade inhibitor (FIG. 7). In some embodiments, methods of the present disclosure comprise a step of administration of an SV-101 formulation, wherein the step of administering SV-101 is a second step in methods described herein. In some embodiments, the step of administering SV-101 is a second step in a method (e.g., a method described herein) that follows a first step of lysing a tumor by cryolysis. In many embodiments of the present disclosure, such a combination of agents facilitates priming of a new immune response, strongly helping with activation and maturation of APCs, while helping with priming of naïve T cells, facilitating their stimulation, proliferation, survival and formation of memory T cells. This combination of active ingredients is intended for predominantly “cold” tumors, that do not show heavy T cell infiltration or that do not show strong expression of PD-L1. In many embodiments of the present disclosure, administration of SV-101 primes a new round of immune responses in tumors that show some T cell infiltration, and that may have characteristics of “hot” tumors, but that have stopped responding to treatment with αPD1 checkpoint inhibitors.

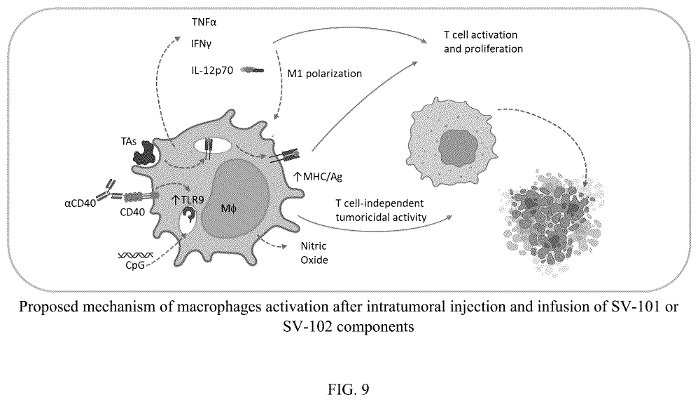

One of the first changes that follow intratumoral cryolysis and intratumoral infusion of SV-101, as described herein, is the recruitment and activation of APCs of different classes such as macrophages, B cells and DCs. The combination of a TLR9 agonist and an αCD40 agonist in a TME can activate these cells in a unique synergistic manner that is not achievable by the use of either agent in isolation. First, a T-cell independent synergistic effect has been observed on the direct tumoricidal activity of macrophages (FIG. 9). Treatment with αCD40 agonistic mAbs can increase the expression of TLR9, which then increases the response to TLR9 agonists. Macrophages stimulated in this way show high levels of expression of TNFα, IFNγ, and IL12p70, which helps to skew their phenotype to pro-inflammatory M1 phenotype. Importantly, the secretion of IL12 is something observed only when both αCD40 and TLR9 agonists are used in combination. Additionally, this synergy leads to an increase in NO production and direct T cell-independent tumoricidal activity. Moreover, the Th1 cytokines secreted leads to an M1 polarization, and increased expression of MEW molecules, which helps TA presentation and increases T cell activation and proliferation.

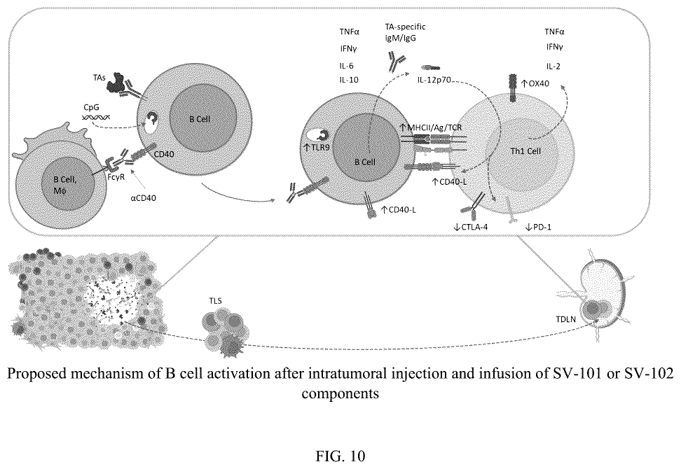

B cells, present in tumors, tertiary lymphoid structures (TLS), and TDLNs are also stimulated by the dual combination of αCD40 mAb and TLR9 agonists (FIG. 10). B cells express CD40 and TLR9 receptors and can bind to TAs via their membrane IgM. Agonism by an αCD40 mAb requires the help of an FcγRIIB-expressing cell, such as a macrophage or another B-cell, to aid in the cross-linking of the CD40 trimers. Agonism of CD40 receptor results in increased expression of TLR9 receptors, which increases the sensitivity to CpG ODNs. The combined stimulus results in increases in expression of TNFα, IFNγ, IL6, IL10, and IL12. Also, there is an increase in the expression of CD40L by B cells which can help and potentiate the agonistic effect of αCD40 mAb and help with activation of pDCs. B cells, stimulated in this way can increase the secretion of TA-specific IgM and IgG. Moreover, B cells can present processed TSA peptides to helper T cells via their MHC-II receptor. Interestingly, B cells are capable of inducing proliferation and Th1 differentiation of allogeneic naïve CD4+ T cells only when exposed to both CpG ODNs and CD40L, via a mechanism that depends on IL12. IL12p70 is a key Th1 cytokine which can lead to changes in helper T cells such as increases in the expression of stimulatory molecules such as CD40-L and OX40, a reduction in immunosuppressive molecules that are associated with T cell stimulation such as CTLA4 and PD1, and an increase of Th1 cytokines such as TNFα, IFNγ and IL-2.

Similar effects of αCD40 and TLR9 agonists are observed in pDCs and cDCs (FIG. 10). The combined activity of these two agents has shown to increase the Th1 cytokines IFNα, IL12p70, TNFα, IL1ß, IL6, which lead to its activation and stimulation of Th1 T cell response and IP10 (or CXCL10), a chemokine that promotes recruitment of immature pDCs and activated T cells. More importantly, the production of significantly higher amounts of IFNα and bioactive IL12p70 is only observed when both an αCD40 agonist and a TLR9 agonist are present, and the downstream Th1 responses are dependent on IL12. Additionally, other relevant phenotypic and functional changes take place on pDCs including: 1) increase in CD40, which is expected to increase their sensitivity to αCD40 agonist and help with stimulation by T helper cells expressing CD40L; 2) increase in OX40L expression, which can help DCs to stimulate and activate T cells which express OX40, leading to increased survival and establishment of memory T cells; 3) increase expression of CCR7, the ligand for CXCL10 chemokine, which favors recruitment and homing of activated DCs to LNs; 4) increase in expression of costimulatory molecules CD80 and CD86, which provide the co-stimulatory signal during T cell priming in TDLN; 5) increase in MHC-II expression, which helps to stimulate CD4+ helper T cells; 6) increase in MHC-I expression and cross-presentation response, which helps with stimulation of cytotoxic CD8+ T cells; 7) increase in the expression of co-stimulatory molecules 4-1BB-L, GITR-L; and 8) downregulation of immuno-suppressive molecule PD-L1.

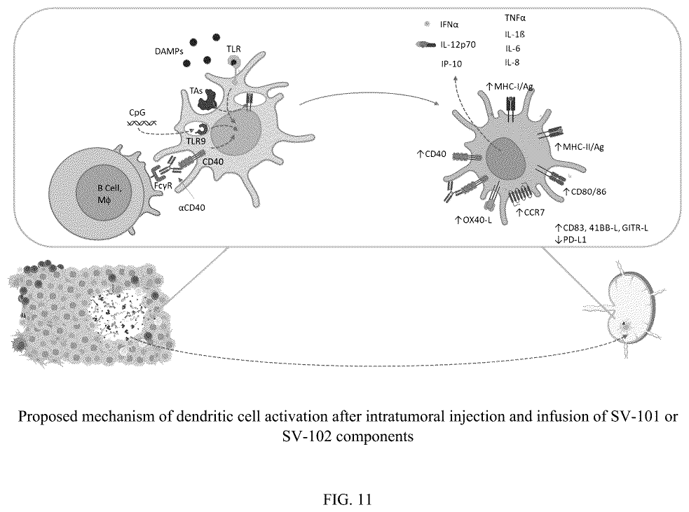

The priming and activation of naïve CD4+ and CD8+ T cells takes place in the TDLN, and to an extend also in TLS in TME (FIG. 11). CD4+ T cells with the appropriate TCR can recognize the processed TA-peptides in the context of the MHC-II (signal 1). Similarly, CD8+ T cells with the right TCR recognize the processed TA-peptides in the context of the MHC-I, thanks to cross-presentation of the externally endocytosed TA (signal 1). Both CD4+ and CD8+ T cells get co-stimulation signals by the interaction from CD28 in the T cell with co-stimulatory receptors CD80 and CD86 on the DC (signal 2). IFNα secreted by the activated DCs provides an additional stimulatory signal (signal 3) to T cells and to DCs themselves. As the activation of T cells proceeds, there are counterregulatory mechanisms that are induced and that prevent the activation and survival of those T cells that have too weak or too strong TCR/MHC/Ag interactions. One of these inducible signals expressed on Teff cells is CTLA4, which is induced when the interaction of TCR/MHC/Ag is too strong or prolonged. CTLA-4 has the function of outcompeting CD28, thus blocking the costimulatory signal provided by CD80/86. This restricts the polyclonality of TCR variants and T cell clones that will be selected, proliferate and survive. Also, CTLA-4 is constitutively expressed at high levels by Tregs, which can also dampen the co-stimulatory signal of CD80/86 by direct blocking the interaction with CD28 and by trans-endocytosis of membrane patches from the DC that contain CD80/86. Increased T cell stimulation will result in increase in the secretion of Th1 cytokines such as TNFα, IL2 and IFNγ, and increase in T cell proliferation (↑CD3/Ki67). Thus, blocking CTLA4 with αCTLA mAb will increase the co-stimulatory signal provided by CD28 to proceed, ultimately allowing T cell clones with strong TCR/MHC/Ag avidity to survive and proliferate, generating increased diversity of TCR variants. In addition, if an αCTLA4 mAb used to block CTLA4 is of the IgG1 isotype (such as YH001), an ADCC-mediated deletion of Tregs that express high levels of CTLA4 may occur, which would be an additional mechanism to help the priming of naïve Teff cells.

Another important mechanistic effect of active agents in SV-101 such as αCD40 agonists and TLR9 agonists is the secretion of IL12, which has the effect of increasing the expression of OX40 and CD40-L on T cells. Increased expression of OX40, will turn CD4 T cells susceptible to the agonistic effects of αOX40 mAb, which has the added effect of upregulation of CD40-L on Th cells, which will facilitate activation of cDCs via CD40.

Also, OX40 is highly expressed on Tregs, and αOX40 mAb agonism can suppress the function of FoxP3, leading to downregulation of CTLA4 and IL10, and promotion of Treg reprogramming to Th17 cells. Additionally, it has been shown that αOX40 of the IgG1 isotype can mediate death of Tregs via ADCC, and that this activity can synergize with the ADCC activity of αCTLA4 mAbs of IgG1 isotype. An overall effect provided by the 4-components present in SV-101 is to increase T cell priming while reducing counterregulatory mechanism, helping to increase CD4 Th cell stimulation, proliferation, survival, and memory differentiation with increased TCR polyclonality.

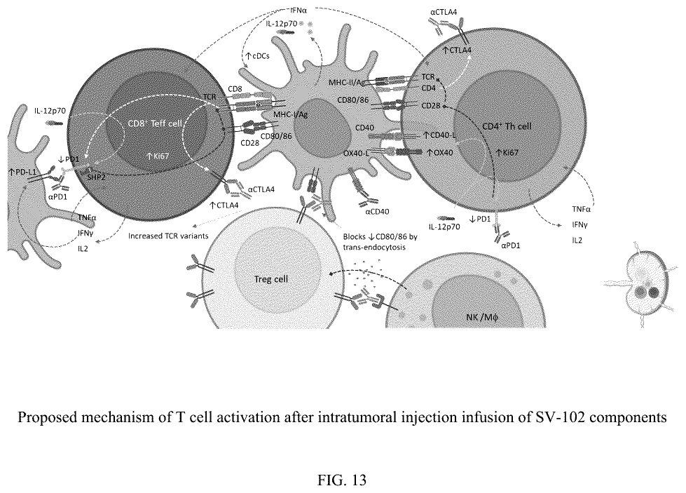

Once activated, CD8+ Teff cells in TDLN proliferate and migrate to the TME (which is further facilitated by the expression of CXCL10 by DCs activated by both TLR9 agonists and αCD40 agonists) to initiate the effector phase of the anti-tumor immune response (FIG. 13).

At a TME, the function of CD8+ Teff cells is to recognize the same TA peptide in the context of the MHC-I and kill the tumor cells by release of GrzB, which reinitiates another cycle of TA and DAMPs from tumor cells to maintain the T cell stimulation cycle. An important immunosuppressive mechanism to overcome in the TME is that mediated by Tregs. Tregs mediate immunosuppression during the effector phase of Teff cells by several mechanisms which include: 1) degradation of the extracellular ATP, which is a DAMP signal, to adenosine by the combined action of membrane enzymes CD39 and CD72; 2) sequestration of immunostimulatory cytokines such as IL-2, by overexpressing the high affinity IL-2R CD25, thus dampening the helper effect of CD4 Th cells; 3) secretion of immunosuppressive cytokines such as TGFß, IL10 and IL35; and 4) direct Teff lysis by secretion of GrzB and perforin. The presence of the αOX40 and αCTLA4 components in SV-101, is also important during the effector phase of the immune response, since these molecules target the function and number of Tregs. Tregs express high levels of both CTLA4 and OX40, and the presence of IgG1 αCTLA and αOX40 can mediate ADCC of Tregs in the TME and reduce the immunosuppressive effects.

In summary, αCD40 agonistic mAbs and TLR9 agonists present in SV-101 mediate a powerful activation and maturation of APCs (mainly immature dendritic cells, but also macrophages, and B cells) in a TME, inducing them to upregulate proinflammatory molecules that favor tumor antigen uptake and cross-presentation (MHC-I, CD80/CD86), as well as cytokines that stimulate helper and effector cytotoxic T cells during antigen presentation (TNFα, INFα/ß, IL-12) while at the same time downregulating immunosuppressive molecules to T cells such as PD-L1. The activation of dendritic cells prompts them to migrate to tumor draining lymph nodes where they activate antigen-specific naïve and memory T cells (both CD4 and CD8), favoring a cytotoxic Th1 antitumor immune response. Activation of effector T cells by APCs is favored by the presence of αCTLA4 mAb, which has the dual function of favoring direct co-stimulation by CD80/86-CD28 interaction on activated T cells that express inhibitory CTLA4, and indirect co-stimulation by limiting the sequestration of CD80/86 by Tregs expressing CTLA4. Additional positive stimulation of effector T cells is obtained by the agonistic function of αOX40 mAb, which can trigger signaling that favors survival and differentiation into memory T cells, while inhibiting the differentiation of Tregs by inhibition of FoxP3 signaling. Additionally, αCTLA4 mAbs and αOX40 mAbs in SV-101 inhibit the pre-existing immunosuppressive mechanisms mediated by Tregs by inhibition of their function and by promoting Treg depletion by ADCC. Reduction of Treg in a TME also prevents the recruitment of MDSCs, thus preventing the re-establishment of a suppressive TME post immunogenic cell death. Moreover, IL-12 elicited by αCD40 mAbs and TLR9 ligands (e.g., agonists) can reduce the expression of PD-1 on exhausted T cells, which can help T cell tumor infiltration by preventing lymphocytes from being excluded from a TME by PD-L1 expressed by a tumor.

In some embodiments, methods of the present disclosure comprise a step of intratumoral administration of SV-102, a combination formulation of immune-activating therapeutic agents comprising a TLR9 agonist, an αCD40 agonist, an αPD1 and αCTLA4 checkpoint blockade inhibitor. In some embodiments, methods of the present disclosure comprise a step of intratumoral administration of an SV-102 combination formulation consisting of a TLR9 agonist, an αCD40 agonist, an αPD1 checkpoint blockade inhibitor and an αCTLA4 checkpoint blockade inhibitor. In some embodiments, methods of the present disclosure comprise a step of administration of an SV-102 formulation, wherein the step of administering SV-102 is a second step in methods described herein. In some embodiments, the step of administering SV-101 is a second step in a method (e.g., a method described herein) that follows a first step of lysing a tumor by cryolysis.

In many embodiments of the present disclosure, such a combination of agents is intended to facilitate the priming of a new immune response, strongly helping with the activation and maturation of APCs, while helping with the priming of naïve T cells, counteracting the immunosuppressive action of Tregs while also facilitating the reinvigoration of pre-existing exhausted Teff cells. This combination of active ingredients is intended for the treatment of predominantly hot tumors that show heavy T cell infiltration, as well as “cold” tumors who have not been previously treated and stopped responding to αPD1 checkpoint inhibitors, or tumors that show a strong expression of PD-L1.

The mechanism of action of SV-102 shares similar immune-pharmacologic effects with SV-101 regarding the activation of macrophages (FIG. 9), B cells (FIG. 10) and dendritic cells (FIG. 11). However, there are unique mechanistic differences between SV-102 and SV-101 in the activation/priming of T cell response, and in the effector phase in the tumor microenvironment.

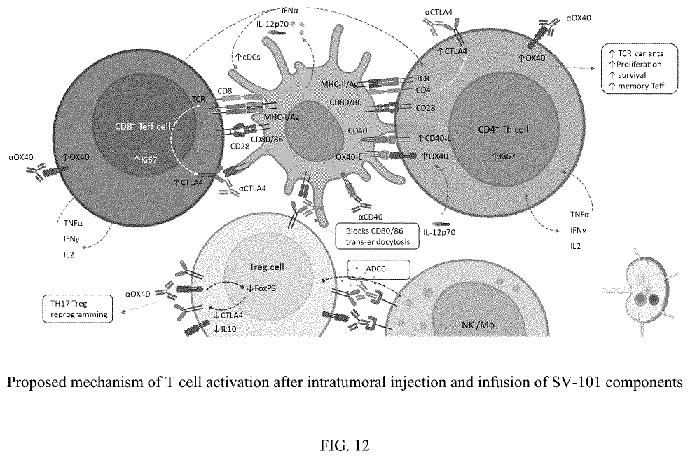

The priming and activation of naïve CD4+ and CD8+ T cells takes place in the TDLN, and to an extend also in TLS in TME (FIG. 12). CD4+ T cells with the appropriate TCR can recognize the processed TA-peptides in the context of the MHC-II (signal 1). Similarly, CD8+ T cells with the right TCR recognize the processed TA-peptides in the context of the MHC-I, thanks to cross-presentation of the externally endocytosed TA (signal 1). Both CD4+ and CD8+ T cells get co-stimulation signals by the interaction from CD28 in the T cell with co-stimulatory receptors CD80 and CD86 on the DC (signal 2). IFNα secreted by the activated DCs provides an additional stimulatory signal (signal 3) to T cells and to DCs themselves. As the activation of T cells proceeds, there are counterregulatory mechanisms that are induced and that prevent the activation and survival of those T cells that have too weak or too strong TCR/MHC/Ag interactions. One of these inducible signals expressed on Teff cells is CTLA4, which is induced when the interaction of TCR/MHC/Ag is too strong or prolonged. CTLA-4 has the function of outcompeting CD28, thus blocking the costimulatory signal provided by CD80/86. This restricts the polyclonality of TCR variants and T cell clones that will be selected, proliferate and survive. Also, CTLA-4 is constitutively expressed at high levels by Tregs, which can also dampen the co-stimulatory signal of CD80/86 by direct blocking the interaction with CD28 and by trans-endocytosis of membrane patches from the DC that contain CD80/86. Increased T cell stimulation will result in increase in the secretion of Th1 cytokines such as TNFα, IL2 and IFNγ, and increase in T cell proliferation (↑CD3/Ki67). Thus, blocking CTLA4 with αCTLA mAb will increase the co-stimulatory signal provided by CD28 to proceed, ultimately allowing T cell clones with strong TCR/MHC/Ag avidity to survive and proliferate, generating increased diversity of TCR variants. In addition, if an αCTLA4 mAb used to block CTLA4 is of the IgG1 isotype, an ADCC-mediated deletion of Tregs that express high levels of CTLA4 may occur, which would be an additional mechanism to help the priming of naïve Teff cells.

Another counterregulatory mechanism induced by sustained TCR activation is the expression of PD1, which can be activated in the TDLN by PD-L1 expressing cells (immature DCs, MDSC, M2 macrophages), which is usually upregulated in response to increased levels of IFNγ. PD-1/PD-L1 interaction will recruit SHP-2 phosphatase which can dampen T cell activation by dephosphorylating the Lck/TCR complex and intracellular domain of CD28. Thus, blocking PD-1/PDL1 interaction with an αPD1 is another mechanism that can help to sustain T cell priming in TDLN.

Another important mechanistic effect of αCD40 agonist and TLR9 agonist agents is the secretion of IL12, which has the effect of lowering the expression of PD-1, thus making them more susceptible to the action of αPD1 mAb, and increasing the expression of OX40 and CD40-L on T cells. When these effects are combined with the increased levels of the cognate ligands OX40-L and CD40 expressed by DCs activated in this way, an increase in CD4 Th cell stimulation and survival, and memory differentiation can be expected.

An overall effect provided by the 4-components present in SV-102 is to increase T cell priming while reducing counterregulatory mechanism, helping to increase CD4 Th cell stimulation, proliferation, survival, and memory differentiation with increased TCR polyclonality.

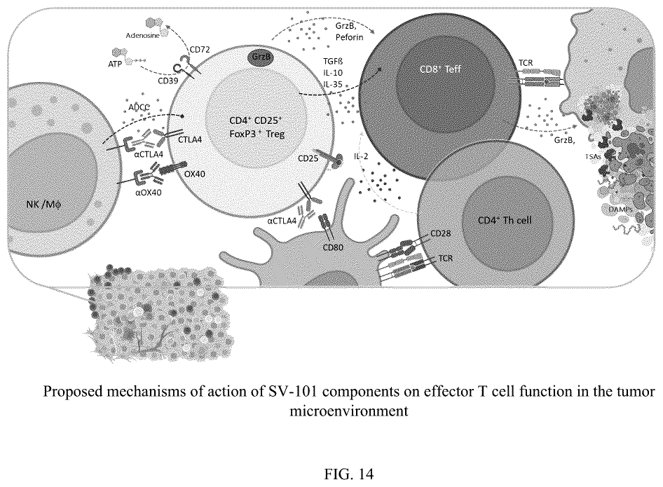

Once activated, CD8+ Teff cells in TDLN proliferate and migrate to the TME (which is further facilitated by the expression of CXCL10 by DCs activated by both TLR9 agonists and αCD40 agonists) to initiate the effector phase of the anti-tumor immune response (FIG. 14).

At a TME, the function of CD8+ Teff cells is to recognize the same TA peptide in the context of the MHC-I and kill the tumor cells by release of GrzB, which reinitiates another cycle of TA and DAMPs from tumor cells to maintain the T cell stimulation cycle.

A consequence of continued T cell stimulation via a TCR is an increase in PD1 expression and release of IFNγ, which leads to induction in the expression of PD-L1 on tumor cells. The PD-1/PD-L1 interaction will result in phosphorylation of PD1 by Lck, which will recruit SHP2 phosphatase, resulting in dephosphorylation of the TCR, and results in exhausted PD1hi Teff, which often are refractory to PD1 checkpoint inhibitors. Exhausted PD1hi T cells also express CD40 in the TME. This exhausted PD1hi T cells, can be reinvigorated by a couple of mechanisms mediated by active agents present in SV-102. First, elevated levels of IL12p70 in the TME (which is achieved by the combined action of αCD40 agonist and TLR9 agonist) can help to lower expression levels of PD1hi to PD1lo, which renders cells more susceptible to αPD1 mAb blocking agents. Also, agonism of CD40 by αCD40 agonist mAb can reinvigorate the metabolism of exhausted T cells by activation of mTORC1.

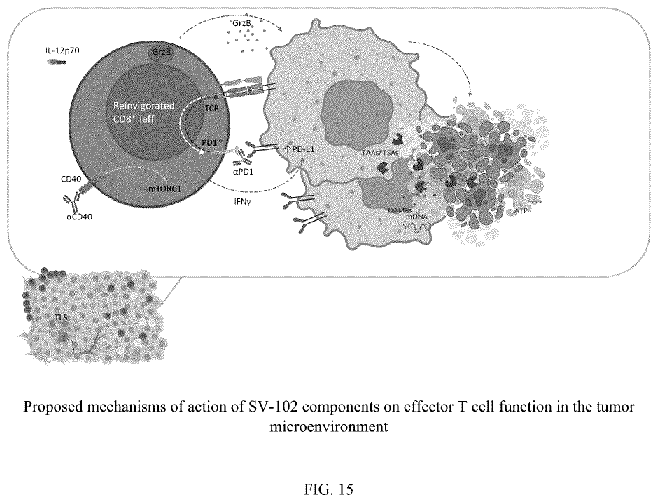

Another important immunosuppressive mechanism to overcome in a TME is a Treg mediated immunosuppressive mechanism (FIG. 15). Tregs mediate immunosuppression during the effector phase of Teff cells by several mechanisms which include: 1) degradation of the extracellular ATP, which is a DAMP signal, to adenosine by the combined action of membrane enzymes CD39 and CD72; 2) sequestration of immunostimulatory cytokines such as IL-2, by overexpressing the high affinity IL-2R CD25, thus dampening the helper effect of CD4 Th cells; 3) secretion of immunosuppressive cytokines such as TGFß, IL10 and IL35; and 4) direct Teff lysis by secretion of GrzB and perforin.

The presence of αCTLA4 in SV-102, is also important during the effector phase of an immune response, since Tregs express high levels of CTLA4, and the presence of IgG1 αCTLA can mediate ADCC of Tregs in the TME and reduce the immunosuppressive effects.

In summary, αCD40 agonistic mAbs and TLR9 agonists present in SV-102 mediate a powerful activation and maturation of APCs (mainly immature dendritic cells, but also macrophages, and B cells) in a TME, inducing them to upregulate proinflammatory molecules that favor tumor antigen uptake and cross-presentation (MHC-I, MHC-II, CD80/CD86), as well as cytokines that stimulate helper and effector cytotoxic T cells during antigen presentation (TNFα, INFα/ß, IL-12) while at the same time downregulating immunosuppressive molecules to T cells such as PD-L1. The activation of dendritic cells prompts them to migrate to tumor draining lymph nodes where they activate antigen-specific naïve and memory T cells (both CD4 and CD8), favoring a cytotoxic Th1 antitumor immune response. Activation of effector T cells by APCs is favored by the presence of αCTLA4 mAb, which has the dual function of favoring direct co-stimulation by CD80/86-CD28 interaction on activated T cells that express inhibitory CTLA4, and indirect co-stimulation by limiting the sequestration of CD80/86 by Tregs expressing CTLA4. Additional positive stimulation of effector T cells is obtained by an αPD1 blocking mAb which can reduce suppressive signals provided by PD-L1+ DCs. Additionally, αCTLA4 mAb in SV-102 inhibits the pre-existing immunosuppressive mechanisms mediated by Tregs by inhibition of their function and by promoting Treg depletion by ADCC. Reduction of Treg in a TME also prevents the recruitment of MDSCs, thus preventing the re-establishment of a suppressive TME post immunogenic cell death. Moreover, IL-12 elicited by αCD40 mAbs and TLR9 ligands (e.g., agonists) can reduce the expression of PD-1 on exhausted T cells, which can help T cell tumor infiltration by preventing lymphocytes from being excluded from a TME by PD-L1 expressed by the tumor and allowing them to become responsive to αPD1 mAb therapy. The presence of αPD1 mAb in SV-102 rescues TILs from exhaustion and can prolong their functional cytotoxic state by preventing them to becoming exhausted.

In addition to the pharmacological activity of individual active ingredients, or agents, injected intratumorally in combination formulations SV-101 and SV-102, the biomechanics of an intratumoral injection and loco-regional infusion play an important role in controlling drug biodistribution, and in mediating biological activity and antitumor efficacy of the methods of the present disclosure. It is a well-known general principle of hydrodynamics that liquids will flow (volume/time) from areas of higher pressure to lower pressure, via the paths of least resistance (change in pressure/flow) and highest compliance (change in volume/change in pressure). During an infusion the tissue exhibits resistance and compliance along a varying scale of elasticity, and the paths of least resistance change as the various resistance and compliance pathways become saturated with fluid and exhibit increasing resistance (back pressure). Almost all normal, healthy tissue primarily uses the lymphatic drainage system to maintain fluid balance within the tissue. The lymphatic drainage vessels are typically the primary drainage pathway for interstitial and extravasated fluid. If the lymphatic drainage is compromised, this results in edema (fluid overload in the tissue). It is also well understood that metastatic tumors are often hyper lymphatic when compared to healthy tissue or to earlier stage tumors and they extensively utilize the lymphatic system to traffic small molecules and macromolecules in and out of a tumor zone to communicate with other tissues such as TDLNs. Moreover, tumors are associated with tertiary lymphoid structures (TLS) either intratumorally, peritumorally or in nearby tissue, and these TLS communicate with the tumor and with secondary lymphoid structures such as TDLNs via lymphatic vessels. The lymphatic tissue drainage system represents an important aspect of the biomechanics of the methods of the present disclosure in that it presents a significant path of least resistance for a tumor tissue resolving an interstitial, extravasated fluid overload (edema) that is created by a drug infusion into the treatment zone. The relatively slow and low pressure (as compared to a subcutaneous or intramuscular injection) infusion rate allows for a drug to first mix (via the closest path of least resistance) with the relatively lower resistance immediately surrounding lysed cell debris in a treatment zone which includes tumor antigens, DAMPS, and other cellular contents. Once the lysed treatment zone has been saturated the infusion will expand via the paths of least resistance into the surrounding intact tumor tissue and also exit via the first available lymphatic vessel drainage pathways, and to a much smaller extent to the blood vessel pathways until the compliance limit of the tissue (tissue's ability to swell to accept additional fluid) is reached at which point the continued infusion pressure will reach an equilibrium where flows out of the tumor primarily via the draining lymphatic vessels and blood vessels are approximately equal to the incoming flow volume being injected into the tumor. This point will vary based on the intratumoral and peritumoral tissue compliance (some tissue is more compliant than others), but it is expected that the equilibrium point will be reached at or before ˜5 mL of a drug (e.g., formulations as described herein) has been infused (based on the limits of tissue compliance associated with intramuscular infusions which vary from 1.5 mL to 5 mL). At the equilibrium point, the continued infusion flow/pressure will have saturated and distended target tumor tissue to the limit of its compliance and infused drug as well as any diffusible entities including the lysed tumor cell contents, tumor antigens, DAMPS, etc. will have potentially mixed with and washed out into the surrounding intratumoral tissue, peritumoral tissue, and lymphatic drainage vessels. Thus, most of this excess fluid mixture of infused drug and some of the mobile entities are expected to drain out of a treatment zone and to reach their cellular targets within a tumor microenvironment, TLSs and TDLNs via the draining lymphatic vessels and into the draining lymph nodes. When the infusion is completed the pressure head of the infusion is terminated and the distended and elastic intratumoral and peritumoral tissues will also drain the mixture of excess drug and debris fluid via the path of least resistance which is expected to primarily be the lymphatic vessel drainage system and return to their resting, pre-injection pressures. It is expected that the remaining mixture of extravasated drug, damaged tissue, intact mobile cells, and cellular debris (tumor antigens, DAMPS, cell fragments, etc.) will be highly immunogenic and is expected to stimulate APC recruitment and activation, migration of APCs to TDLNs, antigen presentation and activation of T cells, and promote cytokines that will favor T cell infiltration into the TME. In addition, the drugs injected in the TME are expected to favor the reinvigoration of exhausted TILs and to block the immunosuppressive function of Tregs, thus shifting the balance to a cytotoxic antitumor immune response.

For at least the reasons described above, in some embodiments, intratumoral injection procedures as described herein, are different from other intratumoral therapies, and should be considered as an intratumoral injection that is aimed at a loco-regional therapy where the injected liquid is not expected to be fully contained within the tumor tissue but is actually expected to enter into lymphoid structures and peritumoral tissue connected to the tumor via lymphatic vessels. In fact, a distinguishing parameter from the method of the present disclosure compared to most intratumoral therapies is that the injected volume of the drug combination exceeds the volume of the cryolysis zone and the volume that could theoretically be contained or retained within the tumor, and extravasation of this liquid composition outside the tumor margins is achieved by injection of a large volume and elevated hydrodynamic pressure.

Thus, it is expected that the methods of the present disclosure create a loco-regional effect at least two physical locations (intratumoral/peritumoral and lymphoid organs) where a drug (e.g., formulations as described herein), antigens, and immune cells are brought into synchronous location and in close proximity, which increases their likelihood of interacting and ultimately leading to a local and systemic antitumor response.

In some embodiments of the present disclosure, a mixture of active ingredients, or agents, in SV-101 or SV-102, is injected intratumorally using the following procedure. After a partial tumor cryolysis treatment zone has been established within a selected tumor, the next step is for a coaxial cryoprobe/infusion needle system (SCINS) to be advanced under precision imaging guidance until the outer coaxial infusion needle tip is residing in the approximate center of a cryolysis treatment zone. Once this is accomplished, the cryoprobe is withdrawn from the SCINS, which leaves the outer coaxial infusion needle tip residing approximately in the center of the cryolysis treatment zone in the tumor and prepared for the drug infusion.

In some embodiments of the present disclosure, combination formulation SV-101 or combination formulation SV-102 is prepared for infusion in a volume of about 1 mL to about mL. In some embodiments, combination formulations are prepared in a volume of about 1 mL to about 5 mL, about 1 mL to about 10 mL, about 1 mL to about 15 mL, about 1 mL to about 20 mL, about 1 mL to about 25 mL, about 5 mL to about 30 mL, about 10 mL to about 30 mL, about mL to about 30 mL, about 5 mL to about 10 mL, about 10 mL to about 15 mL, about 15 mL to about 20 mL, or about 20 mL to about 30 mL. In a preferred embodiment, combination formulations are prepared in a volume of about 15 mL.

In some embodiments of the present disclosure, a syringe containing a drug (e.g., a formulation as described herein) is installed on a pump, a low priming volume connecting tube (for example, MEDLINE 60″ Extension Set w/ microbore tubing) is connected via a one-way stopcock to the syringe and the system is primed with drug by advancing the syringe pump until drug is visualized exiting the connecting tube. The primed connecting tube is then attached to a SCINS infusion needle. The priming volume of the connecting tube is about 0.6 ml. The infusion syringe pump is set to a flow rate of about 1-5 mL/min, with a preferred value of 3 mL/min. No over pressure alarm is set. The pump is then started with the entire volume of about 15 ml being injected in 5 minutes. Attention is paid to ensure that the infusion needle tip is still correctly positioned and that no drug is refluxing back along the needle tract. When the drug infusion pump has completed its infusion cycle and delivered the 15 ml out of the syringe and into the connecting tube, the one-way stopcock is closed. At this point there is about 0.6 ml of residual volume of drug residing in the connecting tube. This volume of drug should now be advanced by removing the one-way stopcock from the infusion syringe and by injecting 0.6 ml of sterile water into the one-way stopcock.

In some embodiments, an infusion syringe pump is set to flow at a rate of about 1 mL/min, about 2 mL/min, about 3 mL/min, about 4 mL/min, or about 5 mL/min. In some embodiments, an infusion syringe pump is set to flow at a rate of about 1 mL/min. In some embodiments, an infusion syringe pump is set to flow at a rate of about 2 mL/min. In some embodiments, an infusion syringe pump is set to flow at a rate of about 3 mL/min. In some embodiments, an infusion syringe pump is set to flow at a rate of about 4 mL/min. In some embodiments, an infusion syringe pump is set to flow at a rate of about 5 mL/min.

In some embodiments of the present disclosure, a TLR9 agonist is a CpG oligodeoxynucleotide (ODN) of class B or C. In some embodiments of the present disclosure, a TLR9 agonist is a CpG oligodeoxynucleotide (ODN) of class B. In some embodiments of the present disclosure, a TLR9 agonist is a CpG oligodeoxynucleotide (ODN) of class C.

In some embodiments, a TLR9 agonist is a nucleic acid as set forth in SEQ ID NO: 1, SEQ ID NO: 2, or SEQ ID NO: 27. In some embodiments, a TLR9 agonist is a nucleic acid as set forth in SEQ ID NO: 1. In some embodiments, a TLR9 agonist is a nucleic acid as set forth in SEQ ID NO: 2. In some embodiments, a TLR9 agonist is a nucleic acid as set forth in SEQ ID NO: 27.

In some embodiments, a TLR9 agonist is administered in accordance with the present disclosure at a dose within the range of about 0.5 mg to about 10 mg, about 1 mg to about 10 mg, about 2 mg to about 10 mg, about 4 mg to about 10 mg, about 8 mg to about 10 mg, about 0.5 mg to about 8 mg, about 0.5 mg to about 6 mg, about 0.5 mg to about 4 mg, about 0.5 mg to about 2 mg, about 0.5 mg to about 1 mg, about 1 mg to about 4 mg, about 2 mg to about 4 mg, or about 3 mg to about 4 mg. In some embodiments, a TLR9 agonist is administered at a dose of about 0.5 mg, about 1 mg, about 2 mg, about 3 mg, about 4 mg, about 5 mg, about 7.5 mg, about 10 mg, about 12 mg, or about 15 mg. In some embodiments, a TLR9 agonist is administered at a dose of about 1 mg. In some embodiments, a TLR9 agonist is administered at a dose of about 2 mg. In some embodiments, a TLR9 agonist is administered at a dose of about 3 mg. In some embodiments, a TLR9 agonist is administered at a dose of about 4 mg.

In some embodiments of the present disclosure, an agonistic anti-CD40 monoclonal antibody is one of human IgG2k isotype. In some embodiments, an agonistic anti-CD40 monoclonal antibody comprises a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8.

In some embodiments of the present disclosure, an agonistic anti-CD40 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28. In some embodiments of the present disclosure, an agonistic anti-CD40 monoclonal antibody comprises a heavy chain, wherein the heavy chain comprises an amino acid sequence having at least 90% sequence identity to SEQ ID NO: 28. In some embodiments of the present disclosure, an agonistic anti-CD40 monoclonal antibody comprises a heavy chain, wherein the heavy chain comprises an amino acid sequence having at least 95% sequence identity to SEQ ID NO: 28. In some embodiments of the present disclosure, an agonistic anti-CD40 monoclonal antibody comprises a heavy chain, wherein the heavy chain comprises an amino acid sequence having at least 96% sequence identity to SEQ ID NO: 28. In some embodiments of the present disclosure, an agonistic anti-CD40 monoclonal antibody comprises a heavy chain, wherein the heavy chain comprises an amino acid sequence having at least 97% sequence identity to SEQ ID NO: 28. In some embodiments of the present disclosure, an agonistic anti-CD40 monoclonal antibody comprises a heavy chain, wherein the heavy chain comprises an amino acid sequence having at least 98% sequence identity to SEQ ID NO: 28. In some embodiments of the present disclosure, an agonistic anti-CD40 monoclonal antibody comprises a heavy chain, wherein the heavy chain comprises an amino acid sequence having at least 99% sequence identity to SEQ ID NO: 28. In some embodiments of the present disclosure, an agonistic anti-CD40 monoclonal antibody comprises a heavy chain, wherein the heavy chain comprises an amino acid sequence as set forth in SEQ ID NO: 28.

In some embodiments of the present disclosure, an agonistic anti-CD40 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments of the present disclosure, an agonistic anti-CD40 monoclonal antibody comprises a light chain, wherein the light chain comprises an amino acid sequence having at least 90% sequence identity to SEQ ID NO: 29. In some embodiments of the present disclosure, an agonistic anti-CD40 monoclonal antibody comprises a light chain, wherein the light chain comprises an amino acid sequence having at least 95% sequence identity to SEQ ID NO: 29. In some embodiments of the present disclosure, an agonistic anti-CD40 monoclonal antibody comprises a light chain, wherein the light chain comprises an amino acid sequence having at least 96% sequence identity to SEQ ID NO: 29. In some embodiments of the present disclosure, an agonistic anti-CD40 monoclonal antibody comprises a light chain, wherein the light chain comprises an amino acid sequence having at least 97% sequence identity to SEQ ID NO: 29. In some embodiments of the present disclosure, an agonistic anti-CD40 monoclonal antibody comprises a light chain, wherein the light chain comprises an amino acid sequence having at least 98% sequence identity to SEQ ID NO: 29. In some embodiments of the present disclosure, an agonistic anti-CD40 monoclonal antibody comprises a light chain, wherein the light chain comprises an amino acid sequence having at least 99% sequence identity to SEQ ID NO: 29. In some embodiments of the present disclosure, an agonistic anti-CD40 monoclonal antibody comprises a light chain, wherein the light chain comprises an amino acid sequence as set forth in SEQ ID NO: 29.

In some embodiments of the present disclosure, an agonistic anti-CD40 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 90% sequence identity to SEQ ID NO: 28, and the light chain comprises an amino acid sequence having at least 90% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 95% sequence identity to SEQ ID NO: 28, and the light chain comprises an amino acid sequence having at least 95% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 96% sequence identity to SEQ ID NO: 28, and the light chain comprises an amino acid sequence having at least 96% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 97% sequence identity to SEQ ID NO: 28, and the light chain comprises an amino acid sequence having at least 97% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 98% sequence identity to SEQ ID NO: 28, and the light chain comprises an amino acid sequence having at least 98% sequence identity to SEQ ID NO: 29. In some embodiments of the present disclosure, an agonistic anti-CD40 monoclonal antibody comprises a heavy chain and a light chain, wherein the heavy chain comprises an amino acid sequence having at least 99% sequence identity to SEQ ID NO: 28, and the light chain comprises an amino acid sequence having at least 99% sequence identity to SEQ ID NO: 29. In some embodiments of the present disclosure, an agonistic anti-CD40 monoclonal antibody comprises a heavy chain and a light chain, wherein the heavy chain comprises an amino acid sequence as set forth in SEQ ID NO: 28, and the light chain comprises an amino acid sequence as set forth in SEQ ID NO: 29.

In some embodiments, an anti-CD40 agonist monoclonal antibody is administered in accordance with the present disclosure at a dose within the range of about 0.5 mg to about 10 mg, about 1 mg to about 10 mg, about 2 mg to about 10 mg, about 4 mg to about 10 mg, about 8 mg to about 10 mg, about 0.5 mg to about 8 mg, about 0.5 mg to about 6 mg, about 0.5 mg to about 4 mg, about 0.5 mg to about 2 mg, about 0.5 mg to about 1 mg, about 1 mg to about 8 mg, or about 4 mg to 8 mg. In some embodiments, an anti-CD40 agonist monoclonal antibody is administered at a dose of about 0.5 mg, about 1 mg, about 2 mg, about 3 mg, about 4 mg, about 5 mg, about 7.5 mg, about 10 mg, about 12 mg, about 15 mg, or about 20 mg. In some embodiments, an anti-CD40 agonist monoclonal antibody is administered at a dose of about 1 mg. In some embodiments, an anti-CD40 agonist monoclonal antibody is administered at a dose of about 5 mg. In some embodiments, an anti-CD40 agonist monoclonal antibody is administered at a dose of about 7.5 mg. In some embodiments, an anti-CD40 agonist monoclonal antibody is administered at a dose of about 10 mg.

In some embodiments of the present disclosure, an agonistic anti-OX40 monoclonal antibody is one of human IgG1k isotype. In some embodiments, an agonistic anti-OX40 monoclonal antibody comprises a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14.

In some embodiments of the present disclosure, an agonistic anti-OX40 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30. In some embodiments of the present disclosure, an agonistic anti-OX40 monoclonal antibody comprises a heavy chain, wherein the heavy chain comprises an amino acid sequence having at least 90% sequence identity to SEQ ID NO: 30. In some embodiments of the present disclosure, an agonistic anti-OX40 monoclonal antibody comprises a heavy chain, wherein the heavy chain comprises an amino acid sequence having at least 95% sequence identity to SEQ ID NO: 30. In some embodiments of the present disclosure, an agonistic anti-OX40 monoclonal antibody comprises a heavy chain, wherein the heavy chain comprises an amino acid sequence having at least 96% sequence identity to SEQ ID NO: 30. In some embodiments of the present disclosure, an agonistic anti-OX40 monoclonal antibody comprises a heavy chain, wherein the heavy chain comprises an amino acid sequence having at least 97% sequence identity to SEQ ID NO: 30. In some embodiments of the present disclosure, an agonistic anti-OX40 monoclonal antibody comprises a heavy chain, wherein the heavy chain comprises an amino acid sequence having at least 98% sequence identity to SEQ ID NO: 30. In some embodiments of the present disclosure, an agonistic anti-OX40 monoclonal antibody comprises a heavy chain, wherein the heavy chain comprises an amino acid sequence having at least 99% sequence identity to SEQ ID NO: 30. In some embodiments of the present disclosure, an agonistic anti-OX40 monoclonal antibody comprises a heavy chain, wherein the heavy chain comprises an amino acid sequence as set forth in SEQ ID NO: 30.

In some embodiments of the present disclosure, an agonistic anti-OX40 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31. In some embodiments of the present disclosure, an agonistic anti-OX40 monoclonal antibody comprises a light chain, wherein the light chain comprises an amino acid sequence having at least 90% sequence identity to SEQ ID NO: 31. In some embodiments of the present disclosure, an agonistic anti-OX40 monoclonal antibody comprises a light chain, wherein the light chain comprises an amino acid sequence having at least 95% sequence identity to SEQ ID NO: 31. In some embodiments of the present disclosure, an agonistic anti-OX40 monoclonal antibody comprises a light chain, wherein the light chain comprises an amino acid sequence having at least 96% sequence identity to SEQ ID NO: 31. In some embodiments of the present disclosure, an agonistic anti-OX40 monoclonal antibody comprises a light chain, wherein the light chain comprises an amino acid sequence having at least 97% sequence identity to SEQ ID NO: 31. In some embodiments of the present disclosure, an agonistic anti-OX40 monoclonal antibody comprises a light chain, wherein the light chain comprises an amino acid sequence having at least 98% sequence identity to SEQ ID NO: 31. In some embodiments of the present disclosure, an agonistic anti-OX40 monoclonal antibody comprises a light chain, wherein the light chain comprises an amino acid sequence having at least 99% sequence identity to SEQ ID NO: 31. In some embodiments of the present disclosure, an agonistic anti-OX40 monoclonal antibody comprises a light chain, wherein the light chain comprises an amino acid sequence as set forth in SEQ ID NO: 31.

In some embodiments of the present disclosure, an agonistic anti-OX40 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 90% sequence identity to SEQ ID NO: 30, and the light chain comprises an amino acid sequence having at least 90% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 95% sequence identity to SEQ ID NO: 30, and the light chain comprises an amino acid sequence having at least 95% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 96% sequence identity to SEQ ID NO: 30, and the light chain comprises an amino acid sequence having at least 96% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 97% sequence identity to SEQ ID NO: 30, and the light chain comprises an amino acid sequence having at least 97% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 98% sequence identity to SEQ ID NO: 30, and the light chain comprises an amino acid sequence having at least 98% sequence identity to SEQ ID NO: 31. In some embodiments of the present disclosure, an agonistic anti-OX40 monoclonal antibody comprises a heavy chain and a light chain, wherein the heavy chain comprises an amino acid sequence having at least 99% sequence identity to SEQ ID NO: 30, and the light chain comprises an amino acid sequence having at least 99% sequence identity to SEQ ID NO: 31. In some embodiments of the present disclosure, an agonistic anti-OX40 monoclonal antibody comprises a heavy chain and a light chain, wherein the heavy chain comprises an amino acid sequence as set forth in SEQ ID NO: 30, and the light chain comprises an amino acid sequence as set forth in SEQ ID NO: 31.

In some embodiments, an anti-OX40 agonist monoclonal antibody is administered in accordance with the present disclosure at a dose within the range of about 0.5 mg to about 10 mg, about 1 mg to about 10 mg, about 2 mg to about 10 mg, about 4 mg to about 10 mg, about 8 mg to about 10 mg, about 0.5 mg to about 8 mg, about 0.5 mg to about 6 mg, about 0.5 mg to about 4 mg, about 0.5 mg to about 2 mg, about 0.5 mg to about 1 mg, about 1 mg to about 8 mg, or about 4 mg to 8 mg. In some embodiments, an anti-OX40 agonist monoclonal antibody is administered at a dose of about 0.5 mg, about 1 mg, about 2 mg, about 3 mg, about 4 mg, about 5 mg, about 7.5 mg, about 10 mg, about 12 mg, about 15 mg, or about 20 mg. In some embodiments, an anti-OX40 agonist monoclonal antibody is administered at a dose of about 1 mg. In some embodiments, an anti-OX40 agonist monoclonal antibody is administered at a dose of about 5 mg. In some embodiments, an anti-OX40 agonist monoclonal antibody is administered at a dose of about 7.5 mg. In some embodiments, an anti-OX40 agonist monoclonal antibody is administered at a dose of about 10 mg.

In some embodiments of the present disclosure, an anti-PD1 monoclonal antibody is of the human IgG4k isotype. In some embodiments, an anti-PD1 monoclonal antibody comprises a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20.

In some embodiments of the present disclosure, an anti-PD1 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32. In some embodiments of the present disclosure, an anti-PD1 monoclonal antibody comprises a heavy chain, wherein the heavy chain comprises an amino acid sequence having at least 90% sequence identity to SEQ ID NO: 32. In some embodiments of the present disclosure, an anti-PD1 monoclonal antibody comprises a heavy chain, wherein the heavy chain comprises an amino acid sequence having at least 95% sequence identity to SEQ ID NO: 32. In some embodiments of the present disclosure, an anti-PD1 monoclonal antibody comprises a heavy chain, wherein the heavy chain comprises an amino acid sequence having at least 96% sequence identity to SEQ ID NO: 32. In some embodiments of the present disclosure, an anti-PD1 monoclonal antibody comprises a heavy chain, wherein the heavy chain comprises an amino acid sequence having at least 97% sequence identity to SEQ ID NO: 32. In some embodiments of the present disclosure, an anti-PD1 monoclonal antibody comprises a heavy chain, wherein the heavy chain comprises an amino acid sequence having at least 98% sequence identity to SEQ ID NO: 32. In some embodiments of the present disclosure, an anti-PD1 monoclonal antibody comprises a heavy chain, wherein the heavy chain comprises an amino acid sequence having at least 99% sequence identity to SEQ ID NO: 32. In some embodiments of the present disclosure, an anti-PD1 monoclonal antibody comprises a heavy chain, wherein the heavy chain comprises an amino acid sequence as set forth in SEQ ID NO: 32.

In some embodiments of the present disclosure, an anti-PD1 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33. In some embodiments of the present disclosure, an anti-PD1 monoclonal antibody comprises a light chain, wherein the light chain comprises an amino acid sequence having at least 90% sequence identity to SEQ ID NO: 33. In some embodiments of the present disclosure, an anti-PD1 monoclonal antibody comprises a light chain, wherein the light chain comprises an amino acid sequence having at least 95% sequence identity to SEQ ID NO: 33. In some embodiments of the present disclosure, an anti-PD1 monoclonal antibody comprises a light chain, wherein the light chain comprises an amino acid sequence having at least 96% sequence identity to SEQ ID NO: 33. In some embodiments of the present disclosure, an anti-PD1 monoclonal antibody comprises a light chain, wherein the light chain comprises an amino acid sequence having at least 97% sequence identity to SEQ ID NO: 33. In some embodiments of the present disclosure, an anti-PD1 monoclonal antibody comprises a light chain, wherein the light chain comprises an amino acid sequence having at least 98% sequence identity to SEQ ID NO: 33. In some embodiments of the present disclosure, an anti-PD1 monoclonal antibody comprises a light chain, wherein the light chain comprises an amino acid sequence having at least 99% sequence identity to SEQ ID NO: 33. In some embodiments of the present disclosure, an anti-PD1 monoclonal antibody comprises a light chain, wherein the light chain comprises an amino acid sequence as set forth in SEQ ID NO: 33.

In some embodiments of the present disclosure, an anti-PD1 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33. In some embodiments of the present disclosure, an anti-PD1 monoclonal antibody comprises a heavy chain and a light chain, wherein the heavy chain comprises an amino acid sequence having at least 90% sequence identity to SEQ ID NO: 32, and the light chain comprises an amino acid sequence having at least 90% sequence identity to SEQ ID NO: 33. In some embodiments of the present disclosure, an anti-PD1 monoclonal antibody comprises a heavy chain and a light chain, wherein the heavy chain comprises an amino acid sequence having at least 95% sequence identity to SEQ ID NO: 32, and the light chain comprises an amino acid sequence having at least 95% sequence identity to SEQ ID NO: 33. In some embodiments of the present disclosure, an anti-PD1 monoclonal antibody comprises a heavy chain and a light chain, wherein the heavy chain comprises an amino acid sequence having at least 96% sequence identity to SEQ ID NO: 32, and the light chain comprises an amino acid sequence having at least 96% sequence identity to SEQ ID NO: 33. In some embodiments of the present disclosure, an anti-PD1 monoclonal antibody comprises a heavy chain and a light chain, wherein the heavy chain comprises an amino acid sequence having at least 97% sequence identity to SEQ ID NO: 32, and the light chain comprises an amino acid sequence having at least 97% sequence identity to SEQ ID NO: 33. In some embodiments of the present disclosure, an anti-PD1 monoclonal antibody comprises a heavy chain and a light chain, wherein the heavy chain comprises an amino acid sequence having at least 98% sequence identity to SEQ ID NO: 32, and the light chain comprises an amino acid sequence having at least 98% sequence identity to SEQ ID NO: 33. In some embodiments of the present disclosure, an anti-PD1 monoclonal antibody comprises a heavy chain and a light chain, wherein the heavy chain comprises an amino acid sequence having at least 99% sequence identity to SEQ ID NO: 32, and the light chain comprises an amino acid sequence having at least 99% sequence identity to SEQ ID NO: 33. In some embodiments of the present disclosure, an anti-PD1 monoclonal antibody comprises a heavy chain and a light chain, wherein the heavy chain comprises an amino acid sequence as set forth in SEQ ID NO: 32, and the light chain comprises an amino acid sequence as set forth in SEQ ID NO: 33.

In some embodiments, an anti-PD1 monoclonal antibody is administered in accordance with the present disclosure at a dose within the range of about 1 mg to about 100 mg, about 2 mg to about 100 mg, about 4 mg to about 100 mg, about 8 mg to about 100 mg, about 10 mg to about 100 mg, about 20 mg to about 100 mg, about 30 mg to about 100 mg, about 40 mg to about 100 mg, about 50 mg to about 100 mg, about 70 mg to about 100 mg, about 90 mg to about 100 mg, about 1 mg to about 90 mg, about 1 mg to about 70 mg, about 1 mg to about 50 mg, about 1 mg to about 40 mg, about 1 mg to about 30 mg, about 1 mg to about 20 mg, about 1 mg to about 10 mg, about 3 mg to about 10 mg, 3 mg to about 30 mg, about 3 mg to about 100 mg, or about 10 mg to about 30 mg. In some embodiments, an anti-PD1 monoclonal antibody is administered at a dose of about 0.5 mg, about 1 mg, about 2 mg, about 3 mg, about 4 mg, about 5 mg, about 7.5 mg, about 10 mg, about 10 mg, about 12 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 110 mg, about 120 mg, about 130 mg, about 140 mg, or about 150 mg. In some embodiments, an anti-PD1 monoclonal antibody is administered at a dose of about 3 mg. In some embodiments, an anti-PD1 monoclonal antibody is administered at a dose of about 10 mg. In some embodiments, an anti-PD1 monoclonal antibody is administered at a dose of about 30 mg. In some embodiments, an anti-PD1 monoclonal antibody is administered at a dose of about 80 mg. In some embodiments, an anti-PD1 monoclonal antibody is administered at a dose of about 100 mg.

In some embodiments of the present disclosure, an anti-CTLA4 monoclonal antibody is of the human IgG1k isotype. In some embodiments, an anti-CTLA4 monoclonal antibody comprises a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments of the present disclosure, an anti-CTLA4 monoclonal antibody comprises a heavy chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34. In some embodiments of the present disclosure, an anti-CTLA4 monoclonal antibody comprises a heavy chain, wherein the heavy chain comprises an amino acid sequence having at least 90% sequence identity to SEQ ID NO: 34. In some embodiments of the present disclosure, an anti-CTLA4 monoclonal antibody comprises a heavy chain, wherein the heavy chain comprises an amino acid sequence having at least 95% sequence identity to SEQ ID NO: 34. In some embodiments of the present disclosure, an anti-CTLA4 monoclonal antibody comprises a heavy chain, wherein the heavy chain comprises an amino acid sequence having at least 96% sequence identity to SEQ ID NO: 34. In some embodiments of the present disclosure, an anti-CTLA4 monoclonal antibody comprises a heavy chain, wherein the heavy chain comprises an amino acid sequence having at least 97% sequence identity to SEQ ID NO: 34. In some embodiments of the present disclosure, an anti-CTLA4 monoclonal antibody comprises a heavy chain, wherein the heavy chain comprises an amino acid sequence having at least 98% sequence identity to SEQ ID NO: 34. In some embodiments of the present disclosure, an anti-CTLA4 monoclonal antibody comprises a heavy chain, wherein the heavy chain comprises an amino acid sequence having at least 99% sequence identity to SEQ ID NO: 34. In some embodiments of the present disclosure, an anti-CTLA4 monoclonal antibody comprises a heavy chain, wherein the heavy chain comprises an amino acid sequence as set forth in SEQ ID NO: 34.

In some embodiments of the present disclosure, an anti-CTLA4 monoclonal antibody comprises a light chain, wherein the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35. In some embodiments of the present disclosure, an anti-CTLA4 monoclonal antibody comprises a light chain, wherein the light chain comprises an amino acid sequence having at least 90% sequence identity to SEQ ID NO: 35. In some embodiments of the present disclosure, an anti-CTLA4 monoclonal antibody comprises a light chain, wherein the light chain comprises an amino acid sequence having at least 95% sequence identity to SEQ ID NO: 35. In some embodiments of the present disclosure, an anti-CTLA4 monoclonal antibody comprises a light chain, wherein the light chain comprises an amino acid sequence having at least 96% sequence identity to SEQ ID NO: 35. In some embodiments of the present disclosure, an anti-CTLA4 monoclonal antibody comprises a light chain, wherein the light chain comprises an amino acid sequence having at least 97% sequence identity to SEQ ID NO: 35. In some embodiments of the present disclosure, an anti-CTLA4 monoclonal antibody comprises a light chain, wherein the light chain comprises an amino acid sequence having at least 98% sequence identity to SEQ ID NO: 35. In some embodiments of the present disclosure, an anti-CTLA4 monoclonal antibody comprises a light chain, wherein the light chain comprises an amino acid sequence having at least 99% sequence identity to SEQ ID NO: 35. In some embodiments of the present disclosure, an anti-CTLA4 monoclonal antibody comprises a light chain, wherein the light chain comprises an amino acid sequence as set forth in SEQ ID NO: 35.

In some embodiments of the present disclosure, an anti-CTLA4 monoclonal antibody comprises a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 90% sequence identity to SEQ ID NO: 34, and the light chain comprises an amino acid sequence having at least 90% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 95% sequence identity to SEQ ID NO: 34, and the light chain comprises an amino acid sequence having at least 95% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 96% sequence identity to SEQ ID NO: 34, and the light chain comprises an amino acid sequence having at least 96% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 97% sequence identity to SEQ ID NO: 34, and the light chain comprises an amino acid sequence having at least 97% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 98% sequence identity to SEQ ID NO: 34, and the light chain comprises an amino acid sequence having at least 98% sequence identity to SEQ ID NO: 35. In some embodiments of the present disclosure, an anti-CTLA4 monoclonal antibody comprises a heavy chain and a light chain, wherein the heavy chain comprises an amino acid sequence having at least 99% sequence identity to SEQ ID NO: 34, and the light chain comprises an amino acid sequence having at least 99% sequence identity to SEQ ID NO: 35. In some embodiments of the present disclosure, an anti-CTLA4 monoclonal antibody comprises a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, an anti-CTLA4 monoclonal antibody is administered in accordance with the present disclosure at a dose within the range of about 1 mg to about 50 mg, about 2 mg to about 50 mg, about 4 mg to about 50 mg, about 8 mg to about 50 mg, about 10 mg to about 50 mg, about 20 mg to about 50 mg, about 30 mg to about 50 mg, about 40 mg to about 50 mg, about 1 mg to about 40 mg, about 1 mg to about 30 mg, about 1 mg to about 20 mg, about 1 mg to about 10 mg, about 1 mg to about 5 mg, about 1 mg to about 2 mg, about 5 mg to about 15 mg, about 5 mg to about 40 mg, or about 15 mg to about 40 mg. In some embodiments, an anti-CTLA4 monoclonal antibody is administered at a dose of about 0.5 mg, about 1 mg, about 2 mg, about 3 mg, about 4 mg, about 5 mg, about 7.5 mg, about 10 mg, about 10 mg, about 12 mg, about 15 mg, about 20 mg, about 25 mg, about 30 mg, about 35 mg, about 40 mg, about 45 mg, or about 50 mg. In some embodiments, an anti-CTLA4 monoclonal antibody is administered at a dose of about 1 mg. In some embodiments, an anti-CTLA4 monoclonal antibody is administered at a dose of about 5 mg. In some embodiments, an anti-CTLA4 monoclonal antibody is administered at a dose of about 15 mg. In some embodiments, an anti-CTLA4 monoclonal antibody is administered at a dose of about 40 mg.

In some preferred embodiments of the present disclosure, intratumoral cryolysis of a solid tumor and intratumoral infusion of SV-101 or SV-102 is repeated every 3 to 8 weeks, at the same lesion or at a different metastatic lesion.

In some embodiments, intratumoral cryolysis of a solid tumor and intratumoral infusion of a combination formulation provided by the present disclosure is repeated about every week, about every 2 weeks, about every 3 weeks, about every 4 weeks, about every 5 weeks, about every 6 weeks, about every 7 weeks, about every 8 weeks, about every 9 weeks, about every 10 weeks, about every 11 weeks, about every 12 weeks, about every 13 weeks, about every 14 weeks, about every 15 weeks, or about every 16 weeks. In some embodiments, treatment is repeated about every 3 to 16 weeks, about every 6 to 16 weeks, about every 8 to 16 weeks, about every 10 to 16 weeks, about every 12 to 16 weeks, about every 14 to 16 weeks, about every 3 to 14 weeks, about every 3 to 12 weeks, about every 3 to 10 weeks, about every 3 to 8 weeks, about every 3 to 6 weeks, or about every 3 to 4 weeks. In some embodiments, a treatment is repeated about every 4 to 8 weeks, about every 5 to 8 weeks, about every 6 to 8 weeks, about every 7 to 8 weeks, about every 4 to 7 weeks, about every 4 to 6 weeks, or about every 4 to 5 weeks. In some embodiments, treatment is repeated every 4 to 8 weeks.

In some embodiments of the present disclosure, an SV-101 formulation contains i) TLR9 agonist at dose levels of about 0.5 mg to about 10 mg per dose, ii) anti-CD40 monoclonal antibody at dose levels of about 0.5 mg to about 10 mg per dose, iii) anti-OX40 monoclonal antibody at dose levels of about 0.5 mg to about 10 mg per dose, and iv) anti-CTLA4 monoclonal antibody within the range of about 1 mg to about 50 mg per dose.

In some embodiments, an SV-101 formulation contains TLR9 agonist, anti-CD40 agonistic antibody, anti-OX40 agonistic antibody and anti-CTLA4 antibody at dose levels of about 1 mg, about 1 mg, about 1 mg and about 1 mg, respectively.

In some embodiments, an SV-101 formulation contains TLR9 agonist, anti-CD40 agonistic antibody, anti-OX40 agonistic antibody and anti-CTLA4 antibody at dose levels of about 2 mg, about 5 mg, about 5 mg and about 5 mg, respectively.

In some embodiments, an SV-101 formulation contains TLR9 agonist, anti-CD40 agonistic antibody, anti-OX40 agonistic antibody and anti-CTLA4 antibody at dose levels of about 3 mg, about 7.5 mg, about 7.5 mg and about 15 mg, respectively.

In some embodiments, an SV-101 formulation contains TLR9 agonist, anti-CD40 agonistic antibody, anti-OX40 agonistic antibody and anti-CTLA4 antibody at dose levels of about 4 mg, about 10 mg, about 10 mg and about 40 mg, respectively.

In some embodiments, an SV-101 formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-101 formulation comprises a TLR9 agonist as set forth in SEQ ID NO: 1; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26;

In some embodiments, an SV-101 formulation comprises a TLR9 agonist as set forth in SEQ ID NO: 2; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-101 formulation comprises a TLR9 agonist as set forth in SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an agonistic anti-OX40 monoclonal antibody comprising a heavy chain and a light chain, wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-101 formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29; an agonistic anti-OX40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 30, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 31; and an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, an SV-101 formulation comprises a TLR9 agonist as set forth in SEQ ID NO: 1; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, an SV-101 formulation comprises a TLR9 agonist as set forth in SEQ ID NO: 2; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments, an SV-101 formulation comprises a TLR9 agonist as set forth in SEQ ID NO: 27; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an agonistic anti-OX40 monoclonal antibody comprising a heavy chain and a light chain, wherein the heavy chain comprises an amino acid sequence as set forth in SEQ ID NO: 30, and the light chain comprises an amino acid sequence as set forth in SEQ ID NO: 31; and an anti-CTLA4 monoclonal antibody comprising a heavy chain and a light chain, wherein the heavy chain comprises an amino acid sequence as set forth in SEQ ID NO: 34, and the light chain comprises an amino acid sequence as set forth in SEQ ID NO: 35.

In some embodiments of the present disclosure, an SV-102 formulation contains i) TLR9 agonist at dose levels of about 0.5 mg to about 10 mg per dose, ii) anti-CD40 monoclonal antibody at dose levels of about 0.5 mg to about 10 mg per dose, iii) anti-PD1 monoclonal antibody at dose levels of about 1 mg to 100 mg per dose and iv) anti-CTLA4 monoclonal antibody within the range of about 1 mg to about 50 mg per dose.

In some embodiments, an SV-102 formulation contains TLR9 agonist, anti-CD40 agonistic antibody, anti-PD1 antibody, and anti-CTLA4 antibody at dose levels of about 1 mg, about 1 mg, about 3 mg and about 1 mg, respectively.

In some embodiments, an SV-102 formulation contains TLR9 agonist, anti-CD40 agonistic antibody, anti-PD1 antibody, and anti-CTLA4 antibody at dose levels of about 2 mg, about 5 mg, about 10 mg and about 5 mg, respectively.

In some embodiments, an SV-102 formulation contains TLR9 agonist, anti-CD40 agonistic antibody, anti-PD1 antibody, and anti-CTLA4 antibody at dose levels of about 3 mg, about 7.5 mg, about 30 mg and about 15 mg, respectively.

In some embodiments, an SV-102 formulation contains TLR9 agonist, anti-CD40 agonistic antibody, anti-PD1 antibody, and anti-CTLA4 antibody at dose levels of about 4 mg, about 10 mg, about 80 mg and about 40 mg, respectively.

In some embodiments, an SV-102 formulation contains TLR9 agonist, anti-CD40 agonistic antibody, anti-PD1 antibody, and anti-CTLA4 antibody at dose levels of about 4 mg, about 10 mg, about 100 mg and about 40 mg, respectively.

In some embodiments, an SV-102 formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-102 formulation comprises a TLR9 agonist as set forth in SEQ ID NO: 1; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-102 formulation comprises a TLR9 agonist as set forth in SEQ ID NO: 2; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-102 formulation comprises a TLR9 agonist as set forth in SEQ ID NO: 27; an agonistic anti-CD40 monoclonal antibody comprising a heavy chain and a light chain, wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8; an anti-PD1 monoclonal antibody comprising a heavy chain and a light chain, wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and an anti-CTLA4 monoclonal antibody comprising a heavy chain and a light chain, wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

In some embodiments, an SV-102 formulation comprises a TLR9 agonist as set forth in SEQ ID NO: 1, SEQ ID NO: 2, or SEQ ID NO: 27; an agonistic anti-CD40 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 28, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 29; an anti-PD1 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 32, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 33; and an anti-CTLA4 monoclonal antibody comprising a heavy chain and a light chain, wherein the heavy chain comprises an amino acid sequence having at least 70%, 75%, 80%, 85%, 90%, 91%, 92%, 93%, 94%, 95%, 96%, 97%, 98%, or 99% sequence identity to SEQ ID NO: 34, and the light chain comprises an amino acid sequence having at least 70%, 75%, 80%, 85%, 90%, 91%, 92%, 93%, 94%, 95%, 96%, 97%, 98%, or 99% sequence identity to SEQ ID NO: 35.

In some embodiments, an SV-102 formulation comprises a TLR9 agonist as set forth in SEQ ID NO: 1; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, an SV-102 formulation comprises a TLR9 agonist as set forth in SEQ ID NO: 2; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments, an SV-102 formulation comprises a TLR9 agonist as set forth in SEQ ID NO: 27; an agonistic anti-CD40 monoclonal antibody comprising a heavy chain and a light chain, wherein the heavy chain comprises an amino acid sequence as set forth in SEQ ID NO: 28, and the light chain comprises an amino acid sequence as set forth in SEQ ID NO: 29; an anti-PD1 monoclonal antibody comprising a heavy chain and a light chain, wherein the heavy chain comprises an amino acid sequence as set forth in SEQ ID NO: 32, and the light chain comprises an amino acid sequence as set forth in SEQ ID NO: 33; and an anti-CTLA4 monoclonal antibody comprising a heavy chain and a light chain, wherein the heavy chain comprises an amino acid sequence as set forth in SEQ ID NO: 34, and the light chain comprises an amino acid sequence having as set forth in SEQ ID NO: 35.

In some embodiments of the present disclosure, SV-101 and SV-102 individual components are injected sequentially in different treatment cycles into the same tumor lesion being treated.

In some embodiments, SV-101 and SV-102 individual components are pre-mixed together and injected simultaneously into the tumor lesion to be treated.

In an embodiment of the present disclosure, a combination formulation is administered in a fixed volume within the range of 1 to 30 mL, most preferable 15 mL. In some embodiments, a combination formulation is administered in a fixed volume within the range of about 1 to 30 mL, about 2 to 30 mL, about 5 to 30 mL, about 10 to 30 mL, about 12 to 30 mL, about 14 to 30 mL, about 16 to 30 mL, about 18 to 30 mL, about 20 to 30 mL, about 22 to 30 mL, about 24 to 30 mL, about 26 to 30 mL, about 28 to 30 mL, about 1 to 28 mL, about 1 to 26 mL, about 1 to 24 mL, about 1 to 22 mL, about 1 to 20 mL, about 1 to 18 mL, about 1 to 16 mL, about 1 to 14 mL, about 1 to 12 mL, about 1 to 10 mL, about 1 to 5 mL, about 1 to 2 mL, about 5 to 25 mL, about 10 to 20 mL, about 12 to 16 mL, or about 13 to 14 mL. In some embodiments, a combination formulation is administered in a fixed volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, about 25 mL, or about 30 mL. In some embodiments, a combination formulation is administered in a fixed volume of about 15 mL.

In an embodiment of the present disclosure, a cancer is a solid tumor cancer selected from adenocarcinoma, astrocytoma, bladder cancer, bone sarcoma, breast cancer, cervical cancer, chordoma, colorectal cancer, endometrial cancer, esophageal cancer, glioblastoma, glioma, kidney cancer, liver cancer, medulloblastoma, melanoma, meningioma, mesothelioma, metastatic pituitary carcinoma, prostate cancer, neuroblastoma, non-melanoma skin cancer, non-small cell lung cancer, oral cancer, ovarian cancer, pancreatic cancer, renal cell carcinoma, retinoblastoma, sarcoma, small cell lung cancer, squamous cell carcinoma (including head and neck cancer), stomach cancer, testicular cancer, thyroid cancer, and Wilms tumor

In an embodiment of the present disclosure, the method for the treatment of cancer involving the steps of a) tumor cell lysis and b) intratumoral administration into the same lesion of a combination formulation of four immunotherapeutic active ingredients comprising a TLR9 agonist, an agonistic anti-CD40 monoclonal antibody, an anti-CTLA4 monoclonal antibody and an agonistic anti-OX40 monoclonal antibody or an anti-PD1 monoclonal antibody is repeated every 3 to 16 weeks, with a preferred frequency of 4-8 weeks for a total number of treatments ranging from 1 to 12 treatment cycles, with a preferred 6 cycles if lesions are available for treatment.

In some embodiments, methods of treatment provided herein are repeated for 1 to 12 cycles, 1 to 10 cycles, 1 to 8 cycles, 1 to 6 cycles, 1 to 4 cycles, 1 to 2 cycles, 3 to 12 cycles, 5 to 12 cycles, 7 to 12 cycles, 9 to 12 cycles, or 11 to 12 cycles. In some embodiments, treatment is repeated for 3 to 6 cycles, 3 to 5 cycles, 3 to 4 cycles, 4 to 6 cycles, or 5 to 6 cycles. In some embodiments, treatment is repeated for 3 to 6 cycles. In some embodiments, treatment is repeated for at least 2, 3, 4, 5, 6, 7, 8, 9, 10, 11, or 12 cycles. In some embodiments, treatment is repeated for 2 cycles. In some embodiments, treatment is repeated for 3 cycles. In some embodiments, treatment is repeated for 4 cycles. In some embodiments, treatment is repeated for 5 cycles. In some embodiments, treatment is repeated for 6 cycles.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” or “approximately” is before a quantitative value, the present disclosure also includes the specific quantitative value itself, unless specifically stated otherwise. As used herein, the term “about” or “approximately” refers to a ±10% variation from the nominal value unless otherwise indicated or inferred.

As used herein, singular forms “a,” “an,” and “the” include plural referents unless the context clearly indicates otherwise. Thus, for example, reference to “an antibody” includes a plurality of antibodies and reference to “an antibody” in some embodiments includes multiple antibodies (e.g., antibodies of different type (e.g., class/isotype, light chain type), antibody fragments or engineered antibodies, etc.), and so forth.

The use of the alternative (e.g., “or”) should be understood to mean either one, both, or any combination thereof of the alternatives.

### EXAMPLES

The following examples are provided for illustration and are not in any way to limit the scope of the disclosure.

### Example 1: Preparation of SV-101—Low Dose

The present Example describes preparation of an SV-101 combination formulation. In a sterile 20 mL vial, the following components are mixed and diluted to a volume of 15 mL in sterile saline solution. In one SV-101 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an OX40 antibody is a YH002 antibody (e.g., defined by CDR sequences set forth in SEQ ID NOs: 9-14 and/or VH/VL sequences set forth in SEQ ID NOs: 30-31), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-101 combination formulation, a CpG ODN is of SEQ ID NO: 2 or SEQ ID NO: 27. It will be understood by a skilled artisan that derivatives or variants of the SV-101 components may be used.

| | | | | | | | | | | | | | Total |
|---|---|---|---|---|---|---|---|---|---|---|---|---|---|
| aCTLA4 | | | aOX40 | | | aCD40 | | | CpG ODN | | | Saline | Volume |
| Conc. | Dose | Vol | Conc. | Dose | Vol | Conc. | Dose | Vol | Conc. | Dose | Vol | Vol | Vol |
| mg/mL | mg | mL | mg/ml | mg | mL | mg/mL | mg | mL | mg/mL | mg | mL | mL | mL |
| 10 | 1 | 0.1 | 10 | 1 | 0.1 | 20 | 1 | 0.05 | 8 | 1 | 0.125 | 14.6 | 15.0 |

### Example 2: Preparation of SV-101—Intermediate Dose

The present Example describes preparation of an SV-101 combination formulation. In a sterile 20 mL vial, the following components are mixed and diluted to a volume of 15 mL in sterile saline solution. In one SV-101 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an OX40 antibody is a YH002 antibody (e.g., defined by CDR sequences set forth in SEQ ID NOs: 9-14 and/or VH/VL sequences set forth in SEQ ID NOs: 30-31), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-101 combination formulation, a CpG ODN is of SEQ ID NO: 2 or SEQ ID NO: 27. It will be understood by a skilled artisan that derivatives or variants of the SV-101 components may be used.

| | | | | | | | | | | | | | Total |
|---|---|---|---|---|---|---|---|---|---|---|---|---|---|
| aCTLA4 | | | aOX40 | | | aCD40 | | | CpG ODN | | | Saline | Volume |
| Conc. | Dose | Vol | Conc. | Dose | Vol | Conc. | Dose | Vol | Conc. | Dose | Vol | Vol | Vol |
| mg/mL | mg | ml | mg/mL | mg | mL | mg/mL | mg | mL | mg/mL | mg | mL | mL | mL |
| 10 | 5 | 0.5 | 10 | 5 | 0.5 | 20 | 5 | 0.25 | 8 | 2 | 0.25 | 13.5 | 15.0 |

### Example 3: Preparation of SV-101—High Dose

The present Example describes preparation of an SV-101 combination formulation. In a sterile 20 mL vial, the following components are mixed and diluted to a volume of 15 mL in sterile saline solution. In one SV-101 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an OX40 antibody is a YH002 antibody (e.g., defined by CDR sequences set forth in SEQ ID NOs: 9-14 and/or VH/VL sequences set forth in SEQ ID NOs: 30-31), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-101 combination formulation, a CpG ODN is of SEQ ID NO: 2 or SEQ ID NO: 27. It will be understood by a skilled artisan that derivatives or variants of the SV-101 components may be used.

| | | | | | | | | | | | | | Total |
|---|---|---|---|---|---|---|---|---|---|---|---|---|---|
| aCTLA4 | | | aOX40 | | | aCD40 | | | CpG ODN | | | Saline | Volume |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/mL | mg | mL | mL | mL |
| 10 | 15 | 1.5 | 10 | 7.5 | 0.75 | 20 | 7.5 | 0.375 | 8 | 3 | 0.375 | 12.0 | 15.0 |

### Example 4: Preparation of SV-101—Highest Dose Level

The present Example describes preparation of an SV-101 combination formulation. In a sterile 20 mL vial, the following components are mixed and diluted to a volume of 15 mL in sterile saline solution. In one SV-101 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an OX40 antibody is a YH002 antibody (e.g., defined by CDR sequences set forth in SEQ ID NOs: 9-14 and/or VH/VL sequences set forth in SEQ ID NOs: 30-31), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-101 combination formulation, a CpG ODN is of SEQ ID NO: 2 or SEQ ID NO: 27. It will be understood by a skilled artisan that derivatives or variants of the SV-101 components may be used.

| | | | | | | | | | | | | | Total |
|---|---|---|---|---|---|---|---|---|---|---|---|---|---|
| aCTLA4 | | | aOX40 | | | aCD40 | | | CpG ODN | | | Saline | Volume |
| Conc. | Dose | Vol | Conc. | Dose | Vol | Conc. | Dose | Vol | Conc. | Dose | Vol | Vol | Vol |
| mg/mL | mg | mL | mg/ml | mg | mL | mg/mL | mg | mL | mg/mL | mg | mL | mL | mL |
| 10 | 40 | 4 | 10 | 10 | 1 | 20 | 10 | 0.5 | 8 | 4 | 0.5 | 9.0 | 15.0 |

### Example 5: Preparation of SV-102—Low Dose

The present Example describes preparation of an SV-102 combination formulation. In a sterile 20 mL vial, the following components are mixed and diluted to a volume of 15 mL in sterile saline solution. In one SV-102 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an PD-1 antibody is a 609A antibody (e.g., defined by CDR sequences set forth in SEQ ID NOs: 15-20 and/or VH/VL sequences set forth in SEQ ID NOs: 32-33), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-102 combination formulation, a CpG ODN is of SEQ ID NO: 2 or SEQ ID NO: 27. It will be understood by a skilled artisan that derivatives or variants of the SV-102 components may be used.

| | | | | | | | | | | | | | Total |
|---|---|---|---|---|---|---|---|---|---|---|---|---|---|
| aCTLA4 | | | aPD1 | | | aCD40 | | | CpG ODN | | | Saline | Vol |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/mL | mg | mL | mL | mL |
| 10 | 1 | 0.1 | 25 | 3 | 0.12 | 20 | 1 | 0.05 | 8 | 1 | 0.125 | 14.6 | 15.0 |

### Example 6: Preparation of SV-102—Intermediate Dose

The present Example describes preparation of an SV-102 combination formulation. In a sterile 20 mL vial, the following components are mixed and diluted to a volume of 15 mL in sterile saline solution. In one SV-102 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an PD-1 antibody is a 609A antibody (e.g., defined by CDR sequences set forth in SEQ ID NOs: 15-20 and/or VH/VL sequences set forth in SEQ ID NOs: 32-33), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-102 combination formulation, a CpG ODN is of SEQ ID NO: 2 or SEQ ID NO: 27. It will be understood by a skilled artisan that derivatives or variants of the SV-102 components may be used.

| | | | | | | | | | | | | | Total |
|---|---|---|---|---|---|---|---|---|---|---|---|---|---|
| aCTLA4 | | | aPD1 | | | aCD40 | | | CpG ODN | | | Saline | Vol |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/mL | mg | mL | mL | mL |
| 10 | 5 | 0.5 | 25 | 10 | 0.4 | 20 | 5 | 0.25 | 8 | 2 | 0.25 | 13.6 | 15.0 |

### Example 7: Preparation of SV-102—High Dose

The present Example describes preparation of an SV-102 combination formulation. In a sterile 20 mL vial, the following components are mixed and diluted to a volume of 15 mL in sterile saline solution. In one SV-102 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an PD-1 antibody is a 609A antibody (e.g., defined by CDR sequences set forth in SEQ ID NOs: 15-20 and/or VH/VL sequences set forth in SEQ ID NOs: 32-33), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-102 combination formulation, a CpG ODN is of SEQ ID NO: 2 or SEQ ID NO: 27. It will be understood by a skilled artisan that derivatives or variants of the SV-102 components may be used.

| | | | | | | | | | | | | | Total |
|---|---|---|---|---|---|---|---|---|---|---|---|---|---|
| aCTLA4 | | | aPD1 | | | aCD40 | | | CpG ODN | | | Saline | Vol |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/mL | mg | mL | mL | mL |
| 10 | 15 | 1.5 | 25 | 30 | 1.2 | 20 | 7.5 | 0.375 | 8 | 3 | 0.375 | 11.5 | 15.0 |

### Example 8: Preparation of SV-102—Highest Dose Level

The present Example describes preparation of an SV-102 combination formulation. In a sterile 20 mL vial, the following components (of Table A or Table B) are mixed and diluted to a volume of 15 mL in sterile saline solution. In one SV-102 combination formulation, a CTLA4 antibody is a YH001 antibody (e.g., defined by CDR sequences set forth in SEQ ID NOs: 21-26 and/or VH/VL sequences set forth in SEQ ID NOs: 34-35), an PD-1 antibody is a 609A antibody (e.g., defined by CDR sequences set forth in SEQ ID NOs: 15-20 and/or VH/VL sequences set forth in SEQ ID NOs: 32-33), a CD40 antibody is a YH003 antibody (e.g., defined by CDR sequences set forth in SEQ ID NOs: 3-8 and/or VH/VL sequences set forth in SEQ ID NOs: 28-29), and a CpG ODN is of SEQ ID NO: 1. In another SV-102 combination formulation, a CpG ODN is of SEQ ID NO: 2 or SEQ ID NO: 27. It will be understood by a skilled artisan that derivatives or variants of the SV-102 components may be used.

TABLE A

| | | | | | | | | | | | | | Total |
|---|---|---|---|---|---|---|---|---|---|---|---|---|---|
| aCTLA4 | | | aPD1 | | | aCD40 | | | CpG ODN | | | Saline | Vol |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/mL | mg | mL | mL | mL |
| 10 | 40 | 4 | 25 | 80 | 4 | 20 | 10 | 0.5 | 8 | 4 | 0.5 | 6.0 | 15.0 |

TABLE B

| | | | | | | | | | | | | | Total |
|---|---|---|---|---|---|---|---|---|---|---|---|---|---|
| aCTLA4 | | | aPD1 | | | aCD40 | | | CpG ODN | | | Saline | Vol |
| Conc. | Dose | Vol | Conc. | Dose | Vol | Conc. | Dose | Vol | Conc. | Dose | Vol | Vol | Vol |
| mg/mL | mg | mL | mg/mL | mg | mL | mg/mL | mg | mL | mg/ml | mg | mL | mL | mL |
| 10 | 40 | 4 | 25 | 100 | 4 | 20 | 10 | 0.5 | 8 | 4 | 0.5 | 6.0 | 15.0 |

### Example 9: Treatment Planning and Patient Preparation

The present Example describes exemplary patient screening and treatment criteria. As a preliminary step, detailed imaging studies, including, but not limited to, CT, PET/CT and MRI, are conducted and reviewed for planning purposes to identify potential target tumors to be treated. From the target tumor candidates, a primary or metastatic tumor is selected for therapy, which is assumed to have an antigenic repertoire at least partially representative of the metastatic lesions. The selected tumor can reside in soft tissue, lymph node, or bone, and must be readily accessible via a precision image guided percutaneous approach. The selected tumor is also large enough (e.g., at or greater than 2 cm in size in at least two axes of measurement) in volume and shape to confidently contain an entire cryolysis partial tumor treatment zone within the tumor. Patients are given either general or conscious sedation anesthesia with full monitoring.

### Example 10: Cryolysis Procedure

The present Example describes an exemplary cryolysis procedure. A selected tumor site (e.g., as selected in Example 9) is prepped for a precision image guided percutaneous approach with a coaxial cryoprobe/infusion needle system (SCINS). The SCINS is inserted via standard precision image guided percutaneous technique with the tip of the SCINS being located precisely so that the resulting cryolysis treatment zone volume will reside completely within the selected tumor. The length of the cryolysis zone with the SCINS can be visualized under imaging as length of a cryoprobe that is exposed from the tip of the coaxial infusion needle.

The cryoprobe is inserted into the tumor through the infusion needle. After confirmation of location the infusion needle is pulled back exposing 2.5 cm-3 cm of the distal end of the cryoprobe. The cryolysis treatment is started by pressing a button on the cryo console. This starts the flow of cryogenic fluid through the probe and the beginning of the cooling process. The cryoprobe is cooled such that the probe temperature reaches −40° C. to −60° C. in less than a minute. Cooling is continued for a total time of two minutes (during which, temperature at a cryoprobe may be allowed to reach up to about −120° C. to about −150° C., or colder). An ice ball of approximately 14 mm in diameter is created. This ice ball is visible by typical imaging methods practiced in the field (e.g., ultrasound (US), CT, PET/CT, and so forth). At 2 minutes the flow of cryogenic gas is automatically stopped (however, cooling may be performed for up to about 5 minutes or longer depending on the size of the lesion being treated). The ice ball is allowed to thaw passively (no electrical or gas heating). Thawing and dissipation of the ice ball is visible with typical imaging methods practiced in the field as discussed herein. When the ice ball has totally dissipated the infusion needle is advance down the cryoprobe and into the cryolysis treatment zone.

### Example 11: Intratumoral Injection and Infusion of SV-101 or SV-102

The present Example describes an exemplary intratumoral administration of immunotherapeutic combination formulations. After a partial tumor cryolysis treatment zone has been established within a selected tumor (e.g., as in Example 9 and/or Example 10), an infusion needle is advanced (which is part of the coaxial cryoprobe/infusion needle system (SCINS)) under precision imaging guidance until the outer coaxial infusion needle tip is residing in the approximate center of the cryolysis treatment zone.

Once this is accomplished, the cryoprobe is withdrawn from the SCINS, which leaves the outer coaxial infusion needle tip residing approximately in the center of the cryolysis treatment zone in the tumor and prepared for the drug infusion.

The drug formulation SV-101 or SV-102 is prepared for infusion in a final volume of 15 mL and placed in one syringe compatible for use with a syringe infusion pump. The syringe containing the drug is installed on the pump, a low priming volume connecting tube (for example, MEDLINE 60″ Extension Set w/microbore tubing) is connected via a one-way stopcock to the syringe and the system is primed with drug by advancing the syringe pump until drug is visualized exiting the connecting tube. The primed connecting tube is then attached to the SCINS infusion needle. The infusion syringe pump is set to a flow rate of 3 mL/min. No over pressure alarm is set. The pump is then started with the entire volume of 15 ml being injected in 5 minutes.

Attention is paid to ensure that the infusion needle tip is still correctly positioned and that no drug is refluxing back along the needle tract. When the drug infusion pump has completed its infusion cycle and delivered the 15 mL out of the syringe and into the connecting tube, the one-way stopcock is closed. At this point there is a 0.6 mL of residual volume of drug residing in the connecting tube. This volume of drug should now be advanced by removing the one-way stopcock from the infusion syringe and by injecting 0.6 mL of sterile saline into the one-way stopcock.

### Example 12: Treatment of Patient #1 with mCRPC with SV-102

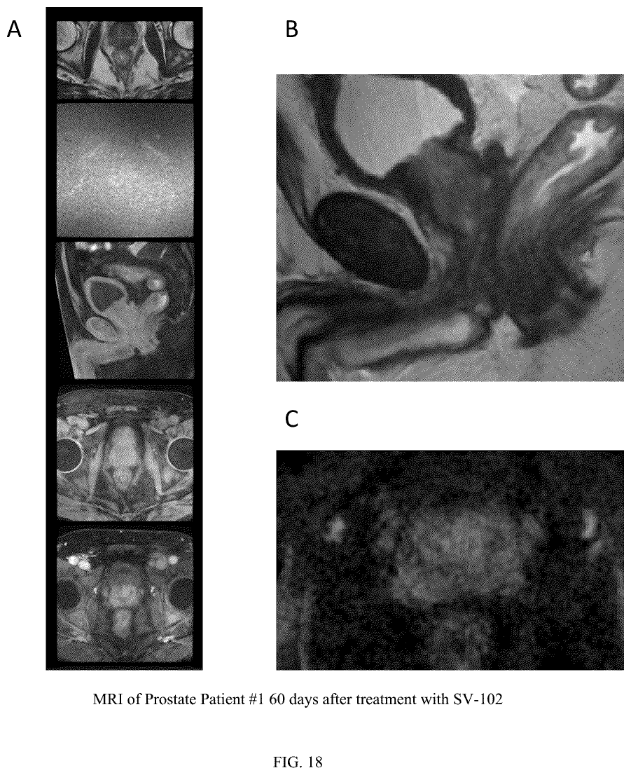

A male patient with metastatic prostate carcinoma confirmed by biopsy and bone scan, had been previously treated with 6 sessions of chemotherapy and hormone blockers (enzalutamide, abiraterone, docetaxel), and radiotherapy of bone metastases. The patient had shown no response and demonstrated disease progression. PET/CT showed a prostatic mass and multiple SUV positive sclerotic bone lesions before treatment. A first treatment of a prostatic lesion was carried out using methods provided by the present disclosure (see, e.g., Examples 9-11), involving a first cycle of cryolysis and injection of SV-102, at high dose level (see, Example 7 and below). After two months of the first treatment, the MRI scan showed no evidence of active disease in the prostate (FIG. 18).

Components of the SV-102 combination formulation included anti-CTLA4 antibody (YH001; CDR sequences set forth in SEQ ID NOs: 21-26 and VH/VL sequences set forth in SEQ ID NOs: 34-35), anti-PD-1 antibody (609A; CDR sequences set forth in SEQ ID NOs: 15-20 and VH/VL sequences set forth in SEQ ID NOs: 32-33), anti-CD40 antibody (YH003; CDR sequences set forth in SEQ ID NOs: 3-8 and VH/VL sequences set forth in SEQ ID NOs: 28-29), and CpG ODN as set forth in SEQ ID NO: 1. Dosage for each component is shown in Example 7.

### Example 13: Treatment of Patient #2 with mCRPC with SV-102

A male patient with metastatic prostate carcinoma confirmed by MRI and prostate biopsy was treated by irreversible electroporation of the prostatic lesion. After 2.5 years, his PSA increased to 12.8 ng/mL and a PET/CT demonstrated a 4×3 cm mass with extracapsular extension. This patient had been previously treated with 10 sessions of radiotherapy of bone metastases, and hormonal therapy (Leuprorelin) for six months. The patient had shown no response and demonstrated more bone metastases by PET/CT and showed a prostatic mass and multiple SUV positive sclerotic bone lesions before treatment. A first treatment of a prostatic lesion was carried out using methods provided by the present disclosure (see, e.g., Examples 9-11), involving a first cycle of cryolysis and injection of SV-102, at high dose level (see, Example 7 and below). At the first follow up exam 5 weeks later, the PSA levels have decreased to 8.56 ng/mL indicating response to the treatment.

Components of the SV-102 combination formulation included anti-CTLA4 antibody (YH001; CDR sequences set forth in SEQ ID NOs: 21-26 and VH/VL sequences set forth in SEQ ID NOs: 34-35), anti-PD-1 antibody (609A; CDR sequences set forth in SEQ ID NOs: 15-20 and VH/VL sequences set forth in SEQ ID NOs: 32-33), anti-CD40 antibody (YH003; CDR sequences set forth in SEQ ID NOs: 3-8 and VH/VL sequences set forth in SEQ ID NOs: 28-29), and CpG ODN as set forth in SEQ ID NO: 1. Dosage for each component is shown in Example 7.

### Example 14: Treatment of Patient #3 with mCRPC with SV-102

A male patient with metastatic prostate carcinoma, confirmed by prostate biopsy (Gleason score 8 and 9) and MRI (5×3 prostatic mass with extracapsular extension) had PSA of 2.97 ng/mL, and no prior treatment. A first treatment of a prostatic lesion was carried out using the methods provided by the present disclosure (see, e.g., Examples 9-11), involving a first cycle of cryolysis and injection of SV-102, at high dose level (see, Example 7 and below). At the first follow up exam 5 weeks later, the PSA levels have decreased to 0.19 ng/mL indicating response to the treatment.

Components of the SV-102 combination formulation included anti-CTLA4 antibody (YH001; CDR sequences set forth in SEQ ID NOs: 21-26 and VH/VL sequences set forth in SEQ ID NOs: 34-35), anti-PD-1 antibody (609A; CDR sequences set forth in SEQ ID NOs: 15-20 and VH/VL sequences set forth in SEQ ID NOs: 32-33), anti-CD40 antibody (YH003; CDR sequences set forth in SEQ ID NOs: 3-8 and VH/VL sequences set forth in SEQ ID NOs: 28-29), and CpG ODN as set forth in SEQ ID NO: 1. Dosage for each component is shown in Example 7.

### Example 15: Treatment of Patient #4 with Metastatic Breast Cancer with SV-101

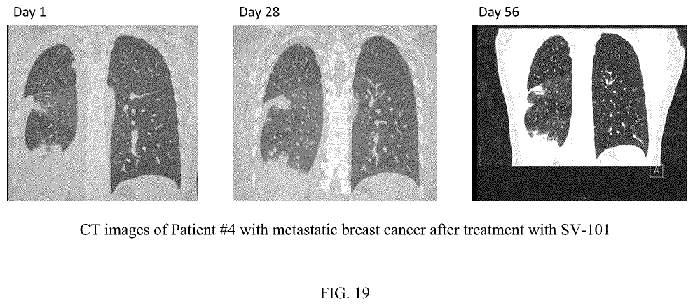

A female patient presented with stage 4 breast cancer with metastasis to lung, pelvis, thorax and lymph nodes (invasive ductal carcinoma, intermediate nuclear grade, ER+, PR+, HER2/NEU-, Ki-67+). The patient had extensive lung metastases, and a large pleural effusion. This patient was treated with the methods provided by the present disclosure (see, e.g., Examples 9-11), with SV-101 at high dose (see, Example 3 and below) on Day 1 in an axillary lymph node, on Day 28 in a subcutaneous lesion and on Day 56 on a different axillary lymph node. A CT scan on Day 28 showed that several lung metastases were resolving, with significant reduction in the axillary nodule and pleural effusion. A CT scan at Day 56 showed that the pleural effusion had resolved, with further reduction in target lesions in the lung, which showed a 40% reduction (Partial Response) based on RECIST criteria compared to baseline measurements (FIG. 19).

Components of the SV-101 combination formulation included anti-CTLA4 antibody (YH001; CDR sequences set forth in SEQ ID NOs: 21-26 and VH/VL sequences set forth in SEQ ID NOs: 34-35), anti-OX40 antibody (YH002; CDR sequences set forth in SEQ ID NOs: 9-14 and VH/VL sequences set forth in SEQ ID NOs: 30-31), anti-CD40 antibody (YH003; CDR sequences set forth in SEQ ID NOs: 3-8 and VH/VL sequences set forth in SEQ ID NOs: 28-29), and CpG ODN as set forth in SEQ ID NO: 1. Dosage for each component is shown in Example 3.

SEQUENCE LISTING

| SEQ ID NO | Description | Sequence |
|---|---|---|
| SEQ ID NO: 1 | LENGTH: 29 | 5′-tcgcgaacgttcgccg |
| | TYPE: DNA | cgtacgtacgcgg-3′ |
| | ORGANISM: | |
| | Artificial Sequence | |
| | FEATURE: | |
| | phosphorothioated | |
| | OTHER INFORMATION: HP007 | |
| | SEQUENCE: 1 | |
| SEQ ID NO: 2 | LENGTH: 22 | 5′-TCGTCGTTTTCGGCG |
| | TYPE: DNA | CGCGCCG-3′ |
| | ORGANISM: | |
| | Artificial Sequence | |
| | FEATURE: | |
| | phosphorothioated | |
| | OTHER INFORMATION: ODN | |
| | 2395 | |
| | SEQUENCE: 2 | |
| SEQ ID NO: 3 | YH003, HC CDR1 | GYTFISYYIY |
| SEQ ID NO: 4 | YH003, HC CDR2 | GINPRNGGTNFNEKFKS |
| SEQ ID NO: 5 | YH003, HC CDR3 | HGNGVY |
| SEQ ID NO: 6 | YH003, LC CDR1 | RSSQSLLHSNGNTYLH |
| SEQ ID NO: 7 | YH003, LC CDR2 | QVSNRFS |
| SEQ ID NO: 8 | YH003, LC CDR3 | SQTTHVPWT |
| SEQ ID NO: 9 | YH002, HC CDR1 | GFSLTSYGVL |
| SEQ ID NO: 10 | YH002, HC CDR2 | VIWSGGSTDYNAAFIS |
| SEQ ID NO: 11 | YH002, HC CDR3 | EEFGY |
| SEQ ID NO: 12 | YH002, LC CDR1 | RASQDINNYLN |
| SEQ ID NO: 13 | YH002, LC CDR2 | YTSRLH |
| SEQ ID NO: 14 | YH002, LC CDR3 | QQTNTLPWT |
| SEQ ID NO: 15 | 609A, HC CDR1 | SYDMS |
| SEQ ID NO: 16 | 609A, HC CDR2 | TISGGGRYTYYPDTVKG |
| SEQ ID NO: 17 | 609A, HC CDR3 | PYGGYFDV |
| SEQ ID NO: 18 | 609A, LC CDR1 | RASQSISNFLH |
| SEQ ID NO: 19 | 609A, LC CDR2 | YASQSIS |
| SEQ ID NO: 20 | 609A, LC CDR3 | QQSNSWPHT |
| SEQ ID NO: 21 | YH001, HC CDR1 | GFTFSSYTMS |
| SEQ ID NO: 22 | YH001, HC CDR2 | SRGGGY |
| SEQ ID NO: 23 | YH001, HC CDR3 | EDYGSSYVHWFAY |
| SEQ ID NO: 24 | YH001, LC CDR1 | RAGENIYSYLA |
| SEQ ID NO: 25 | YH001, LC CDR2 | NARTLAE |
| SEQ ID NO: 26 | YH001, LC CDR3 | QHHYGSPRT |
| SEQ ID NO: 27 | LENGTH: 11-X-11 | 5′-TCG₁AACG₁TTCG₁-X-G₁C |
| | TYPE: DNA | TTG₁CAAG₁CT-5′ |
| | ORGANISM: | where “X” is: |
| | Artificial Sequence | —O—CH₂—CH(OH)—CH₂—O— |
| | FEATURE: phosphorothioated | where G1 is: |
| | OTHER INFORMATION: IMO | 2′-deoxy-7-deazaguanosine |
| | 2125 | |
| | SEQUENCE: 27 | |
| SEQ ID NO: 28 | YH003 | QVQLVQSGAEVKKPGASVKLSCKASGY |
| | Heavy chain | TFISYYIYWVKQAPGQGLEWIGGINPR |
| | | NGGTNFNEKFKSRATLTVDTSISTAYM |
| | | ELSRLRSEDTAVYYCTRHGNGVYWGQG |
| | | TTLTVSSASTKGPSVFPLAPCSRSTSE |
| | | STAALGCLVKDYFPEPVTVSWNSGALT |
| | | SGVHTFPAVLQSSGLYSLSSVVTVPSS |
| | | NFGTQTYTCNVDHKPSNTKVDKTVERK |
| | | CCVECPPCPAPPVAGPSVFLFPPKPKD |
| | | TLMISRTPEVTCVVVDVSHEDPEVQFN |
| | | WYVDGVEVHNAKTKPREEQFNSTFRVV |
| | | SVLTVVHQDWLNGKEYKCKVSNKGLPA |
| | | PIEKTISKTKGQPREPQVYTLPPSREE |
| | | MTKNQVSLTCLVKGFYPSDIAVEWESN |
| | | GQPENNYKTTPPMLDSDGSFFLYSKLT |
| | | VDKSRWQQGNVFSCSVMHEALHNHYTQ |
| | | KSLSLSPGK |
| SEQ ID NO: 29 | YH003 | DVVMTQSPLSLPVTLGQPASISCRSSQ |
| | Light Chain | SLLHSNGNTYLHWYQQRPGQSPNHLIY |
| | | QVSNRFSGVPDRFSGSGSGTDFTLKIS |
| | | RVEAEDVGVYFCSQTTHVPWTFGGGTK |
| | | VEIKRTVAAPSVFIFPPSDEQLKSGTA |
| | | SVVCLLNNFYPREAKVQWKVDNALQSG |
| | | NSQESVTEQDSKDSTYSLSSTLTLSKA |
| | | DYEKHKVYACEVTHQGLSSPVTKSFNR |
| | | GEC |
| SEQ ID NO: 30 | YH002 | QVQLVESGGGVVQPGRSLRISCAVSGF |
| | Heavy Chain | SLTSYGVLWVRQAPGKGLEWLGVIWSG |
| | | GSTDYNAAFISRLTISRDNSKSTVYFQ |
| | | MNSLRAEDTAVYYCAREEFGYWGQGTL |
| | | VTVSSASTKGPSVFPLAPSSKSTSGGT |
| | | AALGCLVKDYFPEPVTVSWNSGALTSG |
| | | VHTFPAVLQSSGLYSLSSVVTVPSSSL |
| | | GTQTYICNVNHKPSNTKVDKKVEPKSC |
| | | DKTHTCPPCPAPELLGGPSVFLFPPKP |
| | | KDTLMISRTPEVTCVVVDVSHEDPEVK |
| | | FNWYVDGVEVHNAKTKPREEQYNSTYR |
| | | VVSVLTVLHQDWLNGKEYKCKVSNKAL |
| | | PAPIEKTISKAKGQPREPQVYTLPPSR |
| | | EEMTKNQVSLTCLVKGFYPSDIAVEWE |
| | | SNGQPENNYKTTPPVLDSDGSFFLYSK |
| | | LTVDKSRWQQGNVFSCSVMHEALHNHY |
| | | TQKSLSLSPGK |
| SEQ ID NO: 31 | YH002 | DIQMTQSPSSLSASVGDRVTITCRASQ |
| | Light Chain | DINNYLNWYQQKPGGAVKLLIYYTSRL |
| | | HTGVPSRFSGSGSGTDFTLTISSLQPE |
| | | DIATYYCQQTNTLPWTFGGGTKLEVKR |
| | | TVAAPSVFIFPPSDEQLKSGTASVVCL |
| | | FLNNYPREAKVQWKVDNALQSGNSQES |
| | | VTEQDSKDSTYSLSSTLTLSKADYEKH |
| | | KVYACEVTHQGLSSPVTKSFNRGEC |
| SEQ ID NO: 32 | 609A | EVKLVESGGGLVQPGGSLRLSCAASG |
| | Heavy Chain | FAFSSYDMSWVRQAPGKRLEWVATIS |
| | | GGGRYTYYPDTVKGRFTISRDNAKNS |
| | | HYLQMNSLRAEDTAVYFCASPYGGYF |
| | | WDVGQGTLVTVSSASTKGPSVFPLAP |
| | | CSRSTSESTAALGCLVKDYFPEPVTV |
| | | SWNSGALTSGVHTFPAVLQSSGLYSL |
| | | SSVVTVPSSSLGTKTYTCNVDHKPSN |
| | | TKVDKRVESKYGPPCPPCPAPEFLGG |
| | | PSVFLFPPKPKDTLMISRTPEVTCVV |
| | | VDVSQEDPEVQFNWYVDGVEVHNAKT |
| | | KPREEQFNSTYRVVSVLTVLHQDWLN |
| | | GKEYKCKVSNKGLPSSIEKTISKAKG |
| | | QPREPQVYTLPPSQEEMTKNQVSLTC |
| | | LVKGFYPSDIAVEWESNGQPENNYKT |
| | | TPPVLDSDGSFFLYSRLTVDKSRWQE |
| | | GNVFSCSVMHEALHNHYTQKSLSLSL |
| | | GK |
| SEQ ID NO: 33 | 609A | EIVLTQSPATLSLSPGERATLSCRAS |
| | Light Chain | QSISNFLHWYQQKPGQAPRLLIKYAS |
| | | QSISGIPARFSGSGSGTDFTLTISSL |
| | | EPEDFAVYFCQQSNSWPHTFGQGTKV |
| | | EIKRTVAAPSVFIFPPSDEQLKSGTA |
| | | SVVCLLNNFYPREAKVQWKVDNALQS |
| | | GNSQESVTEQDSKDSTYSLSSTLTLS |
| | | KADYEKHKVYACEVTHQGLSSPVTKS |
| | | FNRGEC |
| SEQ ID NO: 34 | YH001 | EVQLVESGGGLVQPGGSLRLSCAASG |
| | Heavy Chain | FTFSSYTMSWVRQAPGKGLEWVATIS |
| | | RGGGYTSYPDSVKGRFTISRDNSKNT |
| | | LYLQMNSLRAEDTAVYYCAREDYGSS |
| | | YVHWFAYWGQGTLVTVSAASTKGPSV |
| | | FPLAPSSKSTSGGTAALGCLVKDYFP |
| | | EPVTVSWNSGALTSGVHTFPAVLQSS |
| | | GLYSLSSVVTVPSSSLGTQTYICNVN |
| | | HKPSNTKVDKKVEPKSCDKTHTCPPC |
| | | PAPELLGGPSVFLFPPKPKDTLMISR |
| | | TPEVTCVVVDVSHEDPEVKFNWYVDG |
| | | VEVHNAKTKPREEQYNSTYRVVSVLT |
| | | VLHQDWLNGKEYKCKVSNKALPAPIE |
| | | KTISKAKGQPREPQVYTLPPSREEMT |
| | | KNQVSLTCLVKGFYPSDIAVEWESNG |
| | | QPENNYKTTPPVLDSDGSFFLYSKLT |
| | | VDKSRWQQGNVFSCSVMHEALHNHYT |
| | | QKSLSLSPGK |
| SEQ ID NO: 35 | YH001 | DIQMTQSPSFLSASVGDRVTITCRAG |
| | Light Chain | ENIYSYLAWYQQKPGKAPKLLIYNAR |
| | | TLAEGVPSRFSGSGSGTEFTLTISSL |
| | | QPEDFATYYCQHHYGSPRTFGGGTKL |
| | | EIKRTVAAPSVFIFPPSDEQLKSGTA |
| | | SVVCLLNNFYPREAKVQWKVDNALQS |
| | | GNSQESVTEQDSKDSTYSLSSTLTLS |
| | | KADYEKHKVYACEVTHQGLSSPVTKS |
| | | FNRGEC |

### REFERENCES

Ablin, R. J. (1972). “Cryoimmunotherapy.” Br Med J 3(5824): 476.
Alves Costa Silva, C., F. Facchinetti, B. Routy and L. Derosa (2020). “New pathways in immune stimulation: targeting OX40.” ESMO Open 5(1).
Aspeslagh, S., S. Postel-Vinay, S. Rusakiewicz, J. C. Soria, L. Zitvogel and A. Marabelle (2016). “Rationale for anti-OX40 cancer immunotherapy.” Eur J Cancer 52: 50-66.
Aznar, M. A., N. Tinari, A. J. Rullan, A. R. Sanchez-Paulete, M. E. Rodriguez-Ruiz and I. Melero (2017). “Intratumoral Delivery of Immunotherapy-Act Locally, Think Globally.” J Immunol 198(1): 31-39.
Bagley, D. H., R. P. Faraci, J. C. Marrone and R. M. Beazley (1974). “Lymphocyte mediated cytotoxicity after cryosurgery of a murine sarcoma.” J Surg Res 17(6): 404-406.
Bastianpillai, C., N. Petrides, T. Shah, S. Guillaumier, H. U. Ahmed and M. Arya (2015). “Harnessing the immunomodulatory effect of thermal and non-thermal ablative therapies for cancer treatment.” Tumour Biol 36(12): 9137-9146.
Biniakewitz, M. D., M. K. Kasler and K. L. Fessele (2021). “Immune-Related Adverse Events in the Older Adult with Cancer Receiving Immune Checkpoint Inhibitor Therapy.” Asia Pac J Oncol Nurs 8(1): 18-24.
Blackwood, C. E. and I. S. Cooper (1972). “Response of experimental tumor systems to cryosurgery.” Cryobiology 9(6): 508-515.
Buchbinder, E. I. and A. Desai (2016). “CTLA-4 and PD-1 Pathways: Similarities, Differences, and Implications of Their Inhibition.” Am J Clin Oncol 39(1): 98-106.
Burocchi, A., P. Pittoni, A. Gorzanelli, M. P. Colombo and S. Piconese (2011). “Intratumor OX40 stimulation inhibits IRF1 expression and IL-10 production by Treg cells while enhancing CD40L expression by effector memory T cells.” Eur J Immunol 41(12): 3615-3626.
Cha, E., M. Klinger, Y. Hou, C. Cummings, A. Ribas, M. Faham and L. Fong (2014). “Improved survival with T cell clonotype stability after anti-CTLA-4 treatment in cancer patients.” Sci Transl Med 6(238): 238ra270.
Chae, Y. K., A. Arya, W. lams, M. R. Cruz, S. Chandra, J. Choi and F. Giles (2018). “Current landscape and future of dual anti-CTLA4 and PD-1/PD-L1 blockade immunotherapy in cancer; lessons learned from clinical trials with melanoma and non-small cell lung cancer (NSCLC).” J Immunother Cancer 6(1): 39.
Chen, D. S. and I. Mellman (2013). “Oncology meets immunology: the cancer-immunity cycle.” Immunity 39(1): 1-10.
Choi, Y., Y. Shi, C. L. Haymaker, A. Naing, G. Ciliberto and J. Hajjar (2020). “T-cell agonists in cancer immunotherapy.” J Immunother Cancer 8(2).
Chowdhury, P. S., K. Chamoto and T. Honjo (2018). “Combination therapy strategies for improving PD-1 blockade efficacy: a new era in cancer immunotherapy.” J Intern Med 283(2): 110-120.
Colombo, M. P. (2017). “On OX40 and PD-1 Combination: Why Should OX40 Be First in Sequence?” Clin Cancer Res 23(20): 5999-6001.
Cooperberg, M. R. and Kim, E. D. (2020). “Cryotherapy in Prostate Cancer.” Medscape emedicine.medscape.com
Croft, M. (2010). “Control of immunity by the TNFR-related molecule OX40 (CD134).” Annu Rev Immunol 28: 57-78.
Curran, M. A., W. Montalvo, H. Yagita and J. P. Allison (2010). “PD-1 and CTLA-4 combination blockade expands infiltrating T cells and reduces regulatory T and myeloid cells within B16 melanoma tumors.” Proc Natl Acad Sci USA 107(9): 4275-4280.
Das, R., R. Verma, M. Sznol, C. S. Boddupalli, S. N. Gettinger, H. Kluger, M. Callahan, J. D. Wolchok, R. Halaban, M. V. Dhodapkar and K. M. Dhodapkar (2015). “Combination therapy with anti-CTLA-4 and anti-PD-1 leads to distinct immunologic changes in vivo.” J Immunol 194(3): 950-959.
den Brok, M. H., R. P. Sutmuller, S. Nierkens, E. J. Bennink, C. Frielink, L. W. Toonen, O. C. Boerman, C. G. Figdor, T. J. Ruers and G. J. Adema (2006). “Efficient loading of dendritic cells following cryo and radiofrequency ablation in combination with immune modulation induces anti-tumour immunity.” Br J Cancer 95(7): 896-905.
den Brok, M. H., R. P. Sutmuller, S. Nierkens, E. J. Bennink, L. W. Toonen, C. G. Figdor, T. J. Ruers and G. J. Adema (2006). “Synergy between in situ cryoablation and TLR9 stimulation results in a highly effective in vivo dendritic cell vaccine.” Cancer Res 66(14): 7285-7292.
den Brok, M. H., R. P. Sutmuller, R. van der Voort, E. J. Bennink, C. G. Figdor, T. J. Ruers and G. J. Adema (2004). “In situ tumor ablation creates an antigen source for the generation of antitumor immunity.” Cancer Res 64(11): 4024-4029.
Deng, J., S. Zhao, X. Zhang, K. Jia, H. Wang, C. Zhou and Y. He (2019). “OX40 (CD134) and OX40 ligand, important immune checkpoints in cancer.” Onco Targets Ther 12: 7347-7353.
Dudek, A. M., A. D. Garg, D. V. Krysko, D. De Ruysscher and P. Agostinis (2013). “Inducers of immunogenic cancer cell death.” Cytokine Growth Factor Rev 24(4): 319-333.
Faraci, R. P., D. H. Bagley, J. Marrone and R. M. Beazley (1975). “In vitro demonstration of cryosurgical augmentation of tumor immunity.” Surgery 77(3): 433-438.
Faraci, R. P., D. H. Bagley, J. C. Marrone, A. S. Ketcham and R. M. Beazley (1975). “Mechanism of heightened tumor immunity induced by cryosurgery.” Am Surg 41(5): 309-311.
Fehlings, M., Y. Simoni, H. L. Penny, E. Becht, C. Y. Loh, M. M. Gubin, J. P. Ward, S. C. Wong, R. D. Schreiber and E. W. Newell (2017). “Checkpoint blockade immunotherapy reshapes the high-dimensional phenotypic heterogeneity of murine intratumoural neoantigen-specific CD8(+) T cells.” Nat Commun 8(1): 562.
Ferguson, T. A., J. Choi and D. R. Green (2011). “Armed response: how dying cells influence T-cell functions.” Immunol Rev 241(1): 77-88.
Fraser, J. and W. Gill (1967). “Observations on ultra-frozen tissue.” Br J Surg 54(9): 770-776.
Gage, A. A. and J. Baust (1998). “Mechanisms of tissue injury in cryosurgery.” Cryobiology 37(3): 171-186.
Green, D. R., T. Ferguson, L. Zitvogel and G. Kroemer (2009). “Immunogenic and tolerogenic cell death.” Nat Rev Immunol 9(5): 353-363.
Guo, Z., X. Wang, D. Cheng, Z. Xia, M. Luan and S. Zhang (2014). “PD-1 blockade and OX40 triggering synergistically protects against tumor growth in a murine model of ovarian cancer.” PLoS One 9(2): e89350.
Gursel, E. O., M. S. Roberts and R. J. Veenema (1973). “Cryotherapy in advanced prostatic cancer.” Urology 1(5): 392-396.
Hanawa, S. (1993). “[An experimental study on the induction of anti-tumor immunological activity after cryosurgery for liver carcinoma, and the effect of concomitant immunotherapy with OK432].” Nihon Geka Gakkai Zasshi 94(1): 57-65.
Hervas-Stubbs, S., J. L. Perez-Gracia, A. Rouzaut, M. F. Sanmamed, A. Le Bon and I. Melero (2011). “Direct effects of type I interferons on cells of the immune system.” Clin Cancer Res 17(9): 2619-2627.
Hoffmann, N. E. and J. C. Bischof (2002). “The cryobiology of cryosurgical injury.” Urology 60(2 Suppl 1): 40-49.
Joosten, J. J., G. N. Muijen, T. Wobbes and T. J. Ruers (2001). “In vivo destruction of tumor tissue by cryoablation can induce inhibition of secondary tumor growth: an experimental study.” Cryobiology 42(1): 49-58.
Karapetyan, L., J. J. Luke and D. Davar (2020). “Toll-Like Receptor 9 Agonists in Cancer.” Onco Targets Ther 13: 10039-10060.
Karlsson, J. O., E. G. Cravalho, I. H. Borel Rinkes, R. G. Tompkins, M. L. Yarmush and M. Toner (1993). “Nucleation and growth of ice crystals inside cultured hepatocytes during freezing in the presence of dimethyl sulfoxide.” Biophys J 65(6): 2524-2536.
Keir, M. E., M. J. Butte, G. J. Freeman and A. H. Sharpe (2008). “PD-1 and its ligands in tolerance and immunity.” Annu Rev Immunol 26: 677-704.
Krieg, A. M. (2007). “Development of TLR9 agonists for cancer therapy.” J Clin Invest 117(5): 1184-1194.
Kuang, Z., H. Jing, Z. Wu, J. Wang, Y. Li, H. Ni, P. Zhang, W. Wu, M. Wu, S. Zhou, X. Qiu, D. Wu, B. Prinz, H. Baruah, B. Chen, M. Yu and J. Liu (2020). “Development and characterization of a novel anti-OX40 antibody for potent immune activation.” Cancer Immunol Immunother 69(6): 939-950.
Kvistborg, P., D. Philips, S. Kelderman, L. Hageman, C. Ottensmeier, D. Joseph-Pietras, M. J. Welters, S. van der Burg, E. Kapiteijn, O. Michielin, E. Romano, C. Linnemann, D. Speiser, C. Blank, J. B. Haanen and T. N. Schumacher (2014). “Anti-CTLA-4 therapy broadens the melanoma-reactive CD8+ T cell response.” Sci Transl Med 6(254): 254ra128.
Littrup, P. J., Jallad, B., Vorugu, V., Littrup, G., Currier, B., George, M., and Herring, D. (2009). “Lethal isotherms of cryoablation in a phantom study: Effects of heat load, probe size and number.” J. Vasc Interv Radiol 20(10): 1343-1351.
Marabelle, A., H. Kohrt, C. Caux and R. Levy (2014). “Intratumoral immunization: a new paradigm for cancer therapy.” Clin Cancer Res 20(7): 1747-1756.
Melero, I., D. M. Berman, M. A. Aznar, A. J. Korman, J. L. Perez Gracia and J. Haanen (2015). “Evolving synergistic combinations of targeted immunotherapies to combat cancer.” Nat Rev Cancer 15(8): 457-472.
Messenheimer, D. J., S. M. Jensen, M. E. Afentoulis, K. W. Wegmann, Z. Feng, D. J. Friedman, M. J. Gough, W. J. Urba and B. A. Fox (2017). “Timing of PD-1 Blockade Is Critical to Effective Combination Immunotherapy with Anti-OX40.” Clin Cancer Res 23(20): 6165-6177.
Muller, L. C., M. Micksche, S. Yamagata and F. Kerschbaumer (1985). “Therapeutic effect of cryosurgery of murine osteosarcoma—influence on disease outcome and immune function.” Cryobiology 22(1): 77-85.
Neel, H. B., 3rd and L. W. DeSanto (1973). “Cryosurgical control of cancer: effects of freeze rates, tumor temperatures, and ischemia.” Ann Otol Rhinol Laryngol 82(5): 716-723.
Ngiow, S. F., A. Young, S. J. Blake, G. R. Hill, H. Yagita, M. W. Teng, A. J. Korman and M. J. Smyth (2016). “Agonistic CD40 mAb-Driven IL12 Reverses Resistance to Anti-PD1 in a T-cell-Rich Tumor.” Cancer Res 76(21): 6266-6277.
Piconese, S., B. Valzasina and M. P. Colombo (2008). “OX40 triggering blocks suppression by regulatory T cells and facilitates tumor rejection.” J Exp Med 205(4): 825-839.
Qureshi, O. S., Y. Zheng, K. Nakamura, K. Attridge, C. Manzotti, E. M. Schmidt, J. Baker, L. E. Jeffery, S. Kaur, Z. Briggs, T. Z. Hou, C. E. Futter, G. Anderson, L. S. Walker and D. M. Sansom (2011). “Trans-endocytosis of CD80 and CD86: a molecular basis for the cell-extrinsic function of CTLA-4.” Science 332(6029): 600-603.
Robert, L., C. Harview, R. Emerson, X. Wang, S. Mok, B. Homet, B. Comin-Anduix, R. C. Koya, H. Robins, P. C. Tumeh and A. Ribas (2014). “Distinct immunological mechanisms of CTLA-4 and PD-1 blockade revealed by analyzing TCR usage in blood lymphocytes.” Oncoimmunology 3: e29244.
Robert, L., J. Tsoi, X. Wang, R. Emerson, B. Homet, T. Chodon, S. Mok, R. R. Huang, A. J. Cochran, B. Comin-Anduix, R. C. Koya, T. G. Graeber, H. Robins and A. Ribas (2014). “CTLA4 blockade broadens the peripheral T-cell receptor repertoire.” Clin Cancer Res 20(9): 2424-2432.
Sabel, M. S. (2009). “Cryo-immunology: a review of the literature and proposed mechanisms for stimulatory versus suppressive immune responses.” Cryobiology 58(1): 1-11.
Sabel, M. S., A. Arora, G. Su and A. E. Chang (2006). “Adoptive immunotherapy of breast cancer with lymph node cells primed by cryoablation of the primary tumor.” Cryobiology 53(3): 360-366.
Sabel, M. S. and M. A. Nehs (2005). “Immunologic approaches to breast cancer treatment.” Surg Oncol Clin N Am 14(1): 1-31, v.
Sabel, M. S., M. A. Nehs, G. Su, K. P. Lowler, J. L. Ferrara and A. E. Chang (2005). “Immunologic response to cryoablation of breast cancer.” Breast Cancer Res Treat 90(1): 97-104.
Sabel, M. S., G. Su, K. A. Griffith and A. E. Chang (2010). “Rate of freeze alters the immunologic response after cryoablation of breast cancer.” Ann Surg Oncol 17(4): 1187-1193.
Schonbeck, U. and P. Libby (2001). “The CD40/CD154 receptor/ligand dyad.” Cell Mol Life Sci 58(1): 4-43.
Shah, T. T., Arbel, U., Foss, S., Zachman, A., Rodney, S., Ahmed, H. U., and Arya, M. (2016). “Modeling Cryotherapy Ice Ball Dimensions and Isotherms in a Novel Gel-based Model to Determine Optimal Cryo-needle Configurations and Settings for Potential Use in Clinical Practice” Urology 91: 234-240.
Shibata, T., T. Yamashita, K. Suzuki, N. Takeichi, M. Micallef, M. Hosokawa, H. Kobayashi, M. Murata and M. Arisue (1998) “Enhancement of experimental pulmonary metastasis and inhibition of subcutaneously transplanted tumor growth following cryosurgery.” Anticancer Res 18(6A): 4443-4448.
Shrimali, R. K., S. Ahmad, V. Verma, P. Zeng, S. Ananth, P. Gaur, R. M. Gittelman, E. Yusko, C. Sanders, H. Robins, S. A. Hammond, J. E. Janik, M. Mkrtichyan, S. Gupta and S. N. Khleif (2017). “Concurrent PD-1 Blockade Negates the Effects of OX40 Agonist Antibody in Combination Immunotherapy through Inducing T-cell Apoptosis.” Cancer Immunol Res 5(9): 755-766.
Shrimali, R. K., J. E. Janik, R. Abu-Eid, M. Mkrtichyan and S. N. Khleif (2015). “Programmed death-1 & its ligands: promising targets for cancer immunotherapy.” Immunotherapy 7(7): 777-792.
Sidana, A. (2014). “Cancer immunotherapy using tumor cryoablation.” Immunotherapy 6(1): 85-93.
Soanes, W. A., R. J. Ablin and M. J. Gonder (1970). “Remission of metastatic lesions following cryosurgery in prostatic cancer: immunologic considerations.” J Urol 104(1): 154-159.
Soanes, W. A., M. J. Gonder and R. J. Ablin (1970). “A possible immuno-cryothermic response in prostatic cancer.” Clin Radiol 21(3): 253-255.
Suek, N., L. F. Campesato, T. Merghoub and D. N. Khalil (2019). “Targeted APC Activation in Cancer Immunotherapy to Enhance the Abscopal Effect.” Front Immunol 10: 604.
Tang, F., X. Du, M. Liu, P. Zheng and Y. Liu (2018). “Anti-CTLA-4 antibodies in cancer immunotherapy: selective depletion of intratumoral regulatory T cells or checkpoint blockade?” Cell Biosci 8: 30.
Urano, M., C. Tanaka, Y. Sugiyama, K. Miya and S. Saji (2003). “Antitumor effects of residual tumor after cryoablation: the combined effect of residual tumor and a protein-bound polysaccharide on multiple liver metastases in a murine model.” Cryobiology 46(3): 238-245.
Vilgelm, A. E., D. B. Johnson and A. Richmond (2016). “Combinatorial approach to cancer immunotherapy: strength in numbers.” J Leukoc Biol 100(2): 275-290.
Vonderheide, R. H. (2020). “CD40 Agonist Antibodies in Cancer Immunotherapy.” Annu Rev Med 71: 47-58.
Vonderheide, R. H. and M. J. Glennie (2013). “Agonistic CD40 antibodies and cancer therapy.” Clin Cancer Res 19(5): 1035-1043.
Wei, S. C., C. R. Duffy and J. P. Allison (2018). “Fundamental Mechanisms of Immune Checkpoint Blockade Therapy.” Cancer Discov 8(9): 1069-1086.
Wei, S. C., J. H. Levine, A. P. Cogdill, Y. Zhao, N. A. S. Anang, M. C. Andrews, P. Sharma, J. Wang, J. A. Wargo, D. Pe'er and J. P. Allison (2017). “Distinct Cellular Mechanisms Underlie Anti-CTLA-4 and Anti-PD-1 Checkpoint Blockade.” Cell 170(6): 1120-1133 e1117.
White, A. L., H. T. Chan, A. Roghanian, R. R. French, C. I. Mockridge, A. L. Tutt, S. V. Dixon, D. Ajona, J. S. Verbeek, A. Al-Shamkhani, M. S. Cragg, S. A. Beers and M. J. Glennie (2011). “Interaction with FcgammaRIIB is critical for the agonistic activity of anti-CD40 monoclonal antibody.” J Immunol 187(4): 1754-1763.
Whittaker, D. K. (1984). “Mechanisms of tissue destruction following cryosurgery.” Ann R Coll Surg Engl 66(5): 313-318.
Winograd, R., K. T. Byrne, R. A. Evans, P. M. Odorizzi, A. R. Meyer, D. L. Bajor, C. Clendenin, B. Z. Stanger, E. E. Furth, E. J. Wherry and R. H. Vonderheide (2015). “Induction of T-cell Immunity Overcomes Complete Resistance to PD-1 and CTLA-4 Blockade and Improves Survival in Pancreatic Carcinoma.” Cancer Immunol Res 3(4): 399-411.
Xu, H., X. Xu, W. Ge, J. Lei and D. Cao (2020). “The association between immune-related adverse events and the prognosis of solid cancer patients treated with immunotherapy: a systematic review and meta-analysis.” Ther Adv Med Oncol 12: 1758835920980546.
Yakkala, C., C. L. Chiang, L. Kandalaft, A. Denys and R. Duran (2019). “Cryoablation and Immunotherapy: An Enthralling Synergy to Confront the Tumors.” Front Immunol 10: 2283.
Yu, X., H. T. C. Chan, H. Fisher, C. A. Penfold, J. Kim, T. Inzhelevskaya, C. I. Mockridge, R. R. French, P. J. Duriez, L. R. Douglas, V. English, J. S. Verbeek, A. L. White, I. Tews, M. J. Glennie and M. S. Cragg (2020). “Isotype Switching Converts Anti-CD40 Antagonism to Agonism to Elicit Potent Antitumor Activity.” Cancer Cell 37(6): 850-866 e857.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel Fc mutations to promote antibody multimerization.” MAbs 9(7): 1129-1142.
Zhang, X., X. Xiao, P. Lan, J. Li, Y. Dou, W. Chen, N. Ishii, S. Chen, B. Xia, K. Chen, E. Taparowsky and X. C. Li (2018). “OX40 Costimulation Inhibits Foxp3 Expression and Treg Induction via BATF3-Dependent and Independent Mechanisms.” Cell Rep 24(3): 607-618.
Zhou, J., G. Wang, Y. Chen, H. Wang, Y. Hua and Z. Cai (2019). “Immunogenic cell death in cancer therapy: Present and emerging inducers.” J Cell Mol Med 23(8): 4854-4865.

1. A method of treating cancer in a subject, the method comprising steps of:
a) administering to a tumor in the subject a cryolysis therapy that causes tumor cell lysis; and
b) administering to the subject a therapeutically effective amount of a combination formulation,
wherein the combination formulation comprises:
a. a TLR9 agonist;
b. an agonistic anti-CD40 monoclonal antibody;
c. an anti-CTLA4 monoclonal antibody; and
d. an agonistic anti-OX40 monoclonal antibody or an anti-PD1 monoclonal antibody.

2. The method of claim 1, wherein the combination formulation comprises:
a. the TLR9 agonist;
b. the agonistic anti-CD40 monoclonal antibody;
c. the agonistic anti-OX40 monoclonal antibody; and
d. the anti-CTLA4 monoclonal antibody.

3. (canceled)

4. The method of claim 1, wherein the combination formulation comprises:
a. the TLR9 agonist;
b. the agonistic anti-CD40 monoclonal antibody;
c. the anti-PD1 monoclonal antibody; and
d. the anti-CTLA4 monoclonal antibody.

5. (canceled)

6. The method of claim 1, wherein the TLR9 agonist, the agonistic anti-CD40 monoclonal antibody, the agonistic anti-OX40 monoclonal antibody or the anti-PD1 monoclonal antibody, and the anti-CTLA4 monoclonal antibody are co-administered.

7. The method of claim 1, wherein the TLR9 agonist, the agonistic anti-CD40 monoclonal antibody, the agonistic anti-OX40 monoclonal antibody or the anti-PD1 monoclonal antibody, and the anti-CTLA4 monoclonal antibody are administered sequentially.

8. The method of claim 1, wherein the TLR9 agonist is administered in a separate composition sequentially with a composition comprising the agonistic anti-CD40 monoclonal antibody, the agonistic anti-OX40 monoclonal antibody or the anti-PD1 monoclonal antibody, and the anti-CTLA4 monoclonal antibody.

9.-12. (canceled)

13. The method of claim 1, wherein administration of the cryolysis therapy comprises at least one, at least two, or at least three cycles of freeze-thaw.

14.-15. (canceled)

16. The method of claim 1, wherein administration of the cryolysis therapy comprises no more than one, no more than two, or no more than three cycles of freeze-thaw.

17. The method of claim 1, wherein the cryolysis therapy is mediated by an intratumoral cryolysis device comprising a cryoprobe for contacting and freezing tumor cells during freeze-thaw cycles.

18.-19. (canceled)

20. The method of claim 17, wherein the intratumoral cryolysis device is set to 100% duty cycle and is operated during freeze-thaw cycles to generate a cryoprobe temperature of −40° C. or colder in less than about one minute followed by a step of passive thawing.

21. The method of claim 20, wherein the generated cryoprobe temperature:
a. is maintained for about two minutes before passive thawing; or
b. continues to cool to a temperature of about −70° C. or colder, about −80° C. or colder, about −90° C. or colder, about −100° C. or colder, about −110° C. or colder, about −120° C. or colder, about −130° C. or colder, about −140° C. or colder, or about −150° C. or colder for a total time of about 5 minutes, about 4.5 minutes, about 4 minutes, about 3.5 minutes, about 3 minutes, about 2.5 minutes, about 2 minutes, about 1.75 minutes, about 1.5 minutes, about 1.25 minutes, about 1 minute, about 50 seconds, about 40 seconds, about 30 seconds, about 20 seconds, or about 10 seconds.

22. The method of claim 17, wherein the cryoprobe is cooled to a temperature that creates an ice ball within the tumor that has a diameter of about 14 mm.

23. (canceled)

24. The method of claim 22, wherein the ice ball comprises an approximately 1 cm diameter region within that has a temperature of approximately −40° C. or colder.

25. The method of claim 1, wherein the cryolysis therapy: a) does not ablate the entire tumor but causes partial tumor cell lysis; and/or b) causes necrotic cell death in a zone of tumor tissue with the zone being approximately 14 mm in diameter.

26. (canceled)

27. The method of claim 1, wherein the TLR9 agonist is a CpG ODN of class B or C.

28.-34. (canceled)

35. The method of claim 1, wherein:
a) the TLR9 agonist comprises a nucleic acid sequence as set forth in SEQ ID NO: 1, SEQ ID NO: 2, or SEQ ID NO: 27;
b) the agonistic anti-CD40 monoclonal antibody comprises a heavy chain and a light chain,
wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and
wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8;
c) the agonistic anti-OX40 monoclonal antibody comprises a heavy chain and a light chain,
wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and
wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14;
d) the anti-PD1 monoclonal antibody comprises a heavy chain and a light chain,
wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and
wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and/or
e) the anti-CTLA4 monoclonal antibody comprises a heavy chain and a light chain,
wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and
wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

36.-38. (canceled)

39. The method of claim 1, wherein the cryolysis therapy and the combination formulation are administered into the same tumor.

40.-43. (canceled)

44. The method of claim 1, wherein the treatment is repeated about every 3 to 12 weeks.

45.-46. (canceled)

47. The method of claim 1, wherein the treatment is repeated for 1 to 6 cycles.

48.-52. (canceled)

53. The method of claim 1, wherein the TLR9 agonist, the anti-CD40 agonistic antibody, the anti-OX40 agonistic antibody, and the anti-CTLA4 antibody are administered at a dose of: a) about 1 mg, about 1 mg, about 1 mg, and about 1 mg, respectively; b) about 2 mg, about 5 mg, about 5 mg, and about 5 mg, respectively; c) about 3 mg, about 7.5 mg, about 7.5 mg, and about 15 mg, respectively; or d) about 4 mg, about 10 mg, about 10 mg, and about 40 mg, respectively.

54.-56. (canceled)

57. The method of claim 1, wherein the TLR9 agonist, the anti-CD40 agonistic antibody, the anti-PD1 antibody, and the anti-CTLA4 antibody are administered at a dose of: a) about 1 mg, about 1 mg, about 3 mg, and about 1 mg, respectively; b) about 2 mg, about 5 mg, about 10 mg, and about 5 mg, respectively; c) about 3 mg, about 7.5 mg, about 30 mg, and about 15 mg, respectively; or d) about 4 mg, about 10 mg, about 80 mg, and about 40 mg, respectively.

58.-60. (canceled)

61. The method of claim 1, wherein the combination formulation is administered in a total volume within the range of about 1 mL to about 30 mL.

62. (canceled)

63. The method of claim 1, wherein the cancer is a solid tumor cancer.

64. The method of claim 63, wherein the solid tumor cancer is selected from adenocarcinoma, astrocytoma, bladder cancer, bone sarcoma, breast cancer, cervical cancer, chordoma, colorectal cancer, endometrial cancer, esophageal cancer, glioblastoma, glioma, kidney cancer, liver cancer, medulloblastoma, melanoma, meningioma, mesothelioma, metastatic pituitary carcinoma, prostate cancer, neuroblastoma, non-melanoma skin cancer, non-small cell lung cancer, oral cancer, ovarian cancer, pancreatic cancer, renal cell carcinoma, retinoblastoma, sarcoma, small cell lung cancer, squamous cell carcinoma (including head and neck cancer), stomach cancer, testicular cancer, thyroid cancer, and Wilms tumor.

65.-67. (canceled)

68. A pharmaceutical composition comprising:
a. a TLR9 agonist;
b. an agonistic anti-CD40 monoclonal antibody;
c. an anti-CTLA4 monoclonal antibody;
d. an agonistic anti-OX40 monoclonal antibody; and
e. pharmaceutical acceptable excipients.

69. (canceled)

70. The pharmaceutical composition of claim 68, wherein:
a. the TLR9 agonist is a nucleic acid as set forth in SEQ ID NO: 1, SEQ ID NO: 2, or SEQ ID NO: 27;
b. the agonistic anti-CD40 monoclonal antibody comprises a heavy chain and a light chain,
wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and
wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8;
c. the agonistic anti-OX40 monoclonal antibody comprises a heavy chain and a light chain,
wherein the heavy chain comprises a heavy chain variable region CDR1 comprising the amino acid sequence set forth in SEQ ID NO: 9, a heavy chain variable region CDR2 comprising the amino acid sequence set forth in SEQ ID NO: 10, and a heavy chain variable region CDR3 comprising the amino acid sequence set forth in SEQ ID NO: 11, and
wherein the light chain comprises a light chain variable region CDR1 comprising the amino acid sequence set forth in SEQ ID NO: 12, a light chain variable region CDR2 comprising the amino acid sequence set forth in SEQ ID NO: 13, and a light chain variable region CDR3 comprising the amino acid sequence set forth in SEQ ID NO: 14; and
d. the anti-CTLA4 monoclonal antibody comprises a heavy chain and a light chain,
wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and
wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

71. The pharmaceutical composition of claim 70, wherein respective doses of TLR9 agonist, anti-CD40 agonistic antibody, anti-OX40 agonistic antibody, and anti-CTLA4 antibody are selected from:
a. about 1 mg, about 1 mg, about 1 mg, and about 1 mg, respectively;
b. about 2 mg, about 5 mg, about 5 mg, and about 5 mg, respectively;
c. about 3 mg, about 7.5 mg, about 7.5 mg, and about 15 mg, respectively; and
d. about 4 mg, about 10 mg, about 10 mg, and about 40 mg, respectively.

72. A pharmaceutical composition comprising:
a. a TLR9 agonist;
b. an agonistic anti-CD40 monoclonal antibody;
c. an anti-CTLA4 monoclonal antibody;
d. an agonistic anti-PD1 monoclonal antibody; and
e. pharmaceutical acceptable excipients.

73. (canceled)

74. The pharmaceutical composition of claim 72, wherein:
a. the TLR9 agonist is a nucleic acid as set forth in SEQ ID NO: 1, SEQ ID NO: 2, or SEQ ID NO: 27;
b. the agonistic anti-CD40 monoclonal antibody comprises a heavy chain and a light chain,
wherein the heavy chain comprises a heavy chain variable region CDR1 comprising the amino acid sequence set forth in SEQ ID NO: 3, a heavy chain variable region CDR2 comprising the amino acid sequence set forth in SEQ ID NO: 4, and a heavy chain variable region CDR3 comprising the amino acid sequence set forth in SEQ ID NO: 5, and
wherein the light chain comprises a light chain variable region CDR1 comprising the amino acid sequence set forth in SEQ ID NO: 6, a light chain variable region CDR2 comprising the amino acid sequence set forth in SEQ ID NO: 7, and a light chain variable region CDR3 comprising the amino acid sequence set forth in SEQ ID NO: 8;
c. the anti-PD1 monoclonal antibody comprises a heavy chain and a light chain,
wherein the heavy chain comprises a heavy chain variable region CDR1 comprising the amino acid sequence set forth in SEQ ID NO: 15, a heavy chain variable region CDR2 comprising the amino acid sequence set forth in SEQ ID NO: 16, and a heavy chain variable region CDR3 comprising the amino acid sequence set forth in SEQ ID NO: 17, and
wherein the light chain comprises a light chain variable region CDR1 comprising the amino acid sequence set forth in SEQ ID NO: 18, a light chain variable region CDR2 comprising the amino acid sequence set forth in SEQ ID NO: 19, and a light chain variable region CDR3 comprising the amino acid sequence set forth in SEQ ID NO: 20; and
d. the anti-CTLA4 monoclonal antibody comprises a heavy chain and a light chain,
wherein the heavy chain comprises a heavy chain variable region CDR1 comprising the amino acid sequence set forth in SEQ ID NO: 21, a heavy chain variable region CDR2 comprising the amino acid sequence set forth in SEQ ID NO: 22, and a heavy chain variable region CDR3 comprising the amino acid sequence set forth in SEQ ID NO: 23, and
wherein the light chain comprises a light chain variable region CDR1 comprising the amino acid sequence set forth in SEQ ID NO: 24, a light chain variable region CDR2 comprising the amino acid sequence set forth in SEQ ID NO: 25, and a light chain variable region CDR3 comprising the amino acid sequence set forth in SEQ ID NO: 26.

75. The pharmaceutical composition of claim 74, wherein respective doses of TLR9 agonist, anti-CD40 agonistic antibody, anti-PD1 antibody and anti-CTLA4 antibody are selected from:
a. about 1 mg, about 1 mg, about 3 mg, and about 1 mg, respectively;
b. about 2 mg, about 5 mg, about 10 mg, and about 5 mg, respectively;
c. about 3 mg, about 7.5 mg, about 30 mg, and about 15 mg, respectively;
d. about 4 mg, about 10 mg, about 80 mg, and about 40 mg, respectively; and
e. about 4 mg, about 10 mg, about 100 mg, and about 40 mg, respectively.

76. A method of treating cancer in a subject, the method comprising steps of:
a) administering to a tumor in the subject a cryolysis therapy that causes partial tumor cell lysis; and
b) administering to the same tumor in step a) a therapeutically effective amount of a combination formulation,
wherein the combination formulation comprises:
a. a TLR9 agonist;
b. an agonistic anti-CD40 monoclonal antibody;
c. an anti-CTLA4 monoclonal antibody; and
d. an agonistic anti-OX40 monoclonal antibody;
wherein the cryolysis therapy is administered prior to administration of the combination formulation.

77. A method of treating cancer in a subject, the method comprising steps of:
a) administering to a tumor in the subject a cryolysis therapy that causes partial tumor cell lysis; and
b) administering to the same tumor in step a) a therapeutically effective amount of a combination formulation,
wherein the combination formulation comprises:
a. a TLR9 agonist;
b. an agonistic anti-CD40 monoclonal antibody;
c. an anti-CTLA4 monoclonal antibody; and
d. an anti-PD1 monoclonal antibody;
wherein the cryolysis therapy is administered prior to administration of the combination formulation.

78.-79. (canceled)


SEQUENCE LISTING (35 sequences)

SEQ ID NO: 1 - DNA, 29 bp, synthetic construct
  source 1..29: mol_type = other DNA
tcgcgaacgttcgccgcgtacgtacgcgg

SEQ ID NO: 2 - DNA, 22 bp, synthetic construct
  source 1..22: mol_type = other DNA
tcgtcgttttcggcgcgcgccg

SEQ ID NO: 3 - protein, 10 aa, synthetic construct
GYTFISYYIY

SEQ ID NO: 4 - protein, 17 aa, synthetic construct
GINPRNGGTNFNEKFKS

SEQ ID NO: 5 - protein, 6 aa, synthetic construct
HGNGVY

SEQ ID NO: 6 - protein, 16 aa, synthetic construct
RSSQSLLHSNGNTYLH

SEQ ID NO: 7 - protein, 7 aa, synthetic construct
QVSNRFS

SEQ ID NO: 8 - protein, 9 aa, synthetic construct
SQTTHVPWT

SEQ ID NO: 9 - protein, 10 aa, synthetic construct
GFSLTSYGVL

SEQ ID NO: 10 - protein, 16 aa, synthetic construct
VIWSGGSTDYNAAFIS

SEQ ID NO: 11 - protein, 5 aa, synthetic construct
EEFGY

SEQ ID NO: 12 - protein, 11 aa, synthetic construct
RASQDINNYLN

SEQ ID NO: 13 - protein, 6 aa, synthetic construct
YTSRLH

SEQ ID NO: 14 - protein, 9 aa, synthetic construct
QQTNTLPWT

SEQ ID NO: 15 - protein, 5 aa, synthetic construct
SYDMS

SEQ ID NO: 16 - protein, 17 aa, synthetic construct
TISGGGRYTYYPDTVKG

SEQ ID NO: 17 - protein, 8 aa, synthetic construct
PYGGYFDV

SEQ ID NO: 18 - protein, 11 aa, synthetic construct
RASQSISNFLH

SEQ ID NO: 19 - protein, 7 aa, synthetic construct
YASQSIS

SEQ ID NO: 20 - protein, 9 aa, synthetic construct
QQSNSWPHT

SEQ ID NO: 21 - protein, 10 aa, synthetic construct
GFTFSSYTMS

SEQ ID NO: 22 - protein, 6 aa, synthetic construct
SRGGGY

SEQ ID NO: 23 - protein, 13 aa, synthetic construct
EDYGSSYVHWFAY

SEQ ID NO: 24 - protein, 11 aa, synthetic construct
RAGENIYSYLA

SEQ ID NO: 25 - protein, 7 aa, synthetic construct
NARTLAE

SEQ ID NO: 26 - protein, 9 aa, synthetic construct
QHHYGSPRT

SEQ ID NO: 27 - DNA, 11 bp, synthetic construct
  source 1..11: mol_type = other DNA
  modified_base 3: mod_base = OTHER; note = 2'-deoxy-7-deazaguanosine
  modified_base 7: mod_base = OTHER; note = 2'-deoxy-7-deazaguanosine
  modified_base 11: mod_base = OTHER; note = 2'-deoxy-7-deazaguanosine
tcgaacgttcg

SEQ ID NO: 28 - protein, 441 aa, synthetic construct
QVQLVQSGAEVKKPGASVKLSCKASGYTFISYYIYWVKQAPGQGLEWIGGINPRNGGTNFNEKFKSRATLTVDTSISTAYMELSRLRSEDTAVYYCTRHGNGVYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 219 aa, synthetic construct
DVVMTQSPLSLPVTLGQPASISCRSSQSLLHSNGNTYLHWYQQRPGQSPNHLIYQVSNRFSGVPDRFSGSGSGTDFTLKISRVEAEDVGVYFCSQTTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 443 aa, synthetic construct
QVQLVESGGGVVQPGRSLRISCAVSGFSLTSYGVLWVRQAPGKGLEWLGVIWSGGSTDYNAAFISRLTISRDNSKSTVYFQMNSLRAEDTAVYYCAREEFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, synthetic construct
DIQMTQSPSSLSASVGDRVTITCRASQDINNYLNWYQQKPGGAVKLLIYYTSRLHTGVPSRFSGSGSGTDFTLTISSLQPEDIATYYCQQTNTLPWTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 444 aa, synthetic construct
EVKLVESGGGLVQPGGSLRLSCAASGFAFSSYDMSWVRQAPGKRLEWVATISGGGRYTYYPDTVKGRFTISRDNAKNSHYLQMNSLRAEDTAVYFCASPYGGYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 214 aa, synthetic construct
EIVLTQSPATLSLSPGERATLSCRASQSISNFLHWYQQKPGQAPRLLIKYASQSISGIPARFSGSGSGTDFTLTISSLEPEDFAVYFCQQSNSWPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 452 aa, synthetic construct
EVQLVESGGGLVQPGGSLRLSCAASGFTFSSYTMSWVRQAPGKGLEWVATISRGGGYTSYPDSVKGRFTISRDNSKNTLYLQMNSLRAEDTAVYYCAREDYGSSYVHWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, synthetic construct
DIQMTQSPSFLSASVGDRVTITCRAGENIYSYLAWYQQKPGKAPKLLIYNARTLAEGVPSRFSGSGSGTEFTLTISSLQPEDFATYYCQHHYGSPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC